SEQUENCE LISTING (85 sequences)

SEQ ID NO: 1 - DNA, 18 bp, Homo sapiens
  misc_feature (1)..(1): n is a, c, g, or t
ncaccataaangtgaaan

SEQ ID NO: 2 - DNA, 18 bp, Homo sapiens
  misc_feature (1)..(1): n is a, c, g, or t
ntttcacntttatggtgn

SEQ ID NO: 3 - DNA, 19 bp, Homo sapiens
cttaccgtaacttgaaagt

SEQ ID NO: 4 - DNA, 12 bp, Homo sapiens
ttgacctaagtg

SEQ ID NO: 5 - DNA, 8 bp, Homo sapiens
atttgcat

SEQ ID NO: 6 - DNA, 8 bp, Homo sapiens
atgcaaat

SEQ ID NO: 7 - DNA, 31 bp, Artificial
  synthetic primers
atgcgcggccgcatttgcatgtcgctatgtg

SEQ ID NO: 8 - DNA, 56 bp, Artificial
  synthetic primers
gatcaagcttcatcaggtggctcccgctgaattggaatccacgcactcagctcgtg

SEQ ID NO: 9 - DNA, 34 bp, Artificial
  synthetic primers
atgcgcggccgcaactgatgtatgattatatctt

SEQ ID NO: 10 - DNA, 62 bp, Artificial
  synthetic primers
gatcaagcttcatcaggtggctcccgctgaattggaatccattattatctcctttgttctgt

SEQ ID NO: 11 - DNA, 30 bp, Artificial
  synthetic primers
atgcgcggccgcacagctgtagcagatgct

SEQ ID NO: 12 - DNA, 59 bp, Artificial
  synthetic primers
gatcaagcttcatcaggtggctcccgctgaattggaatccaccacacttggtcaactat

SEQ ID NO: 13 - DNA, 30 bp, Artificial
  synthetic primers
atgcgcggccgcttctcacctaaaggagtc

SEQ ID NO: 14 - DNA, 60 bp, Artificial
  synthetic primers
gatcaagcttcatcaggtggctcccgctgaattggaatccttctaatcctcctaagatca

SEQ ID NO: 15 - DNA, 30 bp, Artificial
  synthetic primers
atgcgcggccgcttcactaagatccagtgc

SEQ ID NO: 16 - DNA, 61 bp, Artificial
  synthetic primers
gatcaagcttcatcaggtggctcccgctgaattggaatccgattcatgaacacagaatatt

SEQ ID NO: 17 - DNA, 33 bp, Artificial
  synthetic primers
atgcgcggccgcgttgaacatttaactctgtat

SEQ ID NO: 18 - DNA, 58 bp, Artificial
  synthetic primers
gatcaagcttcatcaggtggctcccgctgaattggaatccctcatggcacttggagat

SEQ ID NO: 19 - DNA, 20 bp, Artificial
  synthetic primers
ggaaatcttaccttcctgcc

SEQ ID NO: 20 - DNA, 20 bp, Artificial
  synthetic primers
tggctaggtcatgtgaccat

SEQ ID NO: 21 - DNA, 20 bp, Artificial
  synthetic primers
ggaaatcttaccttcctgcc

SEQ ID NO: 22 - DNA, 25 bp, Artificial
  synthetic primers
ttcattcattcattcattgattcac

SEQ ID NO: 23 - DNA, 25 bp, Artificial
  synthetic primers
cagctgcagcagatgctagcagggc

SEQ ID NO: 24 - DNA, 22 bp, Artificial
  synthetic primers
tggctaggtcatgtgaccattc

SEQ ID NO: 25 - DNA, 25 bp, Artificial
  synthetic primers
caatcctcagaaattttcaactgcc

SEQ ID NO: 26 - DNA, 22 bp, Artificial
  synthetic primers
tggctaggtcatgtgaccattc

SEQ ID NO: 27 - DNA, 25 bp, Artificial
  synthetic primers
ctgaaaaagtagtcccagcactttg

SEQ ID NO: 28 - DNA, 27 bp, Artificial
  synthetic primers
atgcggatccgagacagggtcttgctc

SEQ ID NO: 29 - DNA, 28 bp, Artificial
  synthetic primers
atgcggatccgagccaccacacttggtc

SEQ ID NO: 30 - DNA, 22 bp, Artificial
  synthetic primers
gctcacgtagtcccagcacttt

SEQ ID NO: 31 - DNA, 22 bp, Artificial
  synthetic primer
gctcacgtagtcccagcacttt

SEQ ID NO: 32 - DNA, 20 bp, Artificial
  synthetic primer
gctgaggcaggaggatcact

SEQ ID NO: 33 - DNA, 24 bp, Artificial
  synthetic primer
gcactaccacacccagctaatttt

SEQ ID NO: 34 - DNA, 25 bp, Artificial
  synthetic primer
ctgcagaaagaactctctcaacttc

SEQ ID NO: 35 - DNA, 24 bp, Artificial
  synthetic primer
tcaacaattaagtagctggaacca

SEQ ID NO: 36 - DNA, 19 bp, Artificial
  synthetic primer
gaaggtgaaggtcggagtc

SEQ ID NO: 37 - DNA, 20 bp, Artificial
  synthetic primer
gaagatggtgatgggatttc

SEQ ID NO: 38 - DNA, 20 bp, Artificial
  synthetic primer
tacggccataccaccctgaa

SEQ ID NO: 39 - DNA, 20 bp, Artificial
  synthetic primer
gcggtctcccatccaagtac

SEQ ID NO: 40 - DNA, 24 bp, Artificial
  synthetic primer
aagtgtggtggctcaccctgtctc

SEQ ID NO: 41 - DNA, 24 bp, Artificial
  synthetic primer
aagtgagccaccacaccctgtctc

SEQ ID NO: 42 - DNA, 22 bp, Artificial
  synthetic primer
gctcacgtagtcccagcacttt

SEQ ID NO: 43 - DNA, 22 bp, Artificial
  synthetic primer
agaccagcttgggcaacatagt

SEQ ID NO: 44 - DNA, 25 bp, Artificial
  synthetic primer
ctgcagaaagaactctctcaacttc

SEQ ID NO: 45 - DNA, 24 bp, Artificial
  synthetic primer
agttgttaattcatcgaccttggt

SEQ ID NO: 46 - DNA, 30 bp, Artificial
  synthetic primer
agtacctgttttctgcttctcctgtgcagc

SEQ ID NO: 47 - DNA, 19 bp, Artificial
  synthetic primer
gaaggtgaaggtcggagtc

SEQ ID NO: 48 - DNA, 20 bp, Artificial
  synthetic primer
gaagatggtgatgggatttc

SEQ ID NO: 49 - DNA, 20 bp, Artificial
  synthetic primer
caagcttcccgttctcagcc

SEQ ID NO: 50 - DNA, 24 bp, Artificial
  synthetic primer
aagtgtggtggctcaccctgtctc

SEQ ID NO: 51 - DNA, 24 bp, Artificial
  synthetic primer
aagtgagccaccacaccctgtctc

SEQ ID NO: 52 - DNA, 784 bp, Homo sapiens
atttgcatgtcgctatgtgttctgggaaatcaccataaacgtgaaatgtctttggatttgggaatcttataagttctgtatgagaccactttttcccatagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatttttggaaaaaaaaaaaaaaaaaaaaaacaaaacgaaaccgggccgggcgcggtggttcacgcctataatcccagcactttgggaggccgaggcgggcggatcacaaggtcaggaggtcgagaccatccaggctaacacggtgaaacccccccccatctctactaaaaaaaaaaaatacaaaaaattagccattagccgggcgtggtggcgggcgcctataatcccagctacttgggaggctgaagcagaatggcgtgaacccgggaggcggagcttgcagtgagccgagatcgcgccactgcattccagcctgggcgacagagcgagtctcaaaaaaaaaaaaacc

SEQ ID NO: 53 - DNA, 3000 bp, Homo sapiens
tgtattttaaattatgatacataatgactatttaacttccaaacagaattcactcatttacatttatgaacattctgggtataatatccagagggaattaaaccactatctcagagagatatctgcattctgatgttcactgaaacattattcacagtagccaaaatacagaaacaacctgtctgtcaacgaattaatggataaataaaagagataaggaatatatatatacacacacatacacacacaagcacacacacacacatacaatggaaaattattcatcctaaacggaaataaaattctgctatttacaagaagaagaatgaaactggaggaccttctgcttagagaaataagtcagacatagaaagacatatactgcatgatctgacttgtatgtggaatataaaaaagtagaactcatgaaaatagagtagaagggtggttaccagaagttatggggtgggagaaatggagagctattggtccaaggatgcacactttgaatcataaggaataagttctggagacctgatgtgcagtaggatgactatagttaataatcatgtattatatgcttgaaatttgctaagagaatagatattcagtattcttacaacacacacagacacacacacacacaggtatgtcaggtgatggatatgtcaattagcttgattgtggtgatcatttttataatatatacatatatcaaaatagtatatttccagtttttcacttttcttttaatttttattatcatatattttactatataaaatatttttaactaacatgatgtcagtccagcctgaccaacatggagaaaccccatctccactaaaaatacaaaattagctgggcatggtggtgcatgcctgtaatcccagctactggggaggctgaggcaggagaatcattggaacctgggaggcggagtttctggtgagctgagatcacaccattgcactccaatctgggcaacaaaagcaaaattctgccaaaaaaaaaaaattctggacagaattttgcatagaaagccctttttcatccccaaattataataataacatagtacattttctttttgtaattccaagggatctattttttgtttattttgacatatagctcttaggttcttttggcattatttagtgtgtaagagtaagtaaggatatatttttatagttttccaaataatagcaaatatccagaaataacttataaaacaggtcatcctttcaccataaatgtgaaatgctacctttatcctatgtatttgaatatatatacatatatattcaagtacattctctctatatatgtgtgtttataatatcatatatatacacacacatatgtgtgtgtgtgtgtgtgtgtgtgtgttacctctttcaattccatagtgttttaagtaatctaattttggcatactgaaaatactgataagaaaaattcttattttttctttcaaaatttcctttgcatttataatacattaattttccagataatctttagaatcagcttatcaaactttgttagaagtgtattttatgttaatcgagacaatactggagcttgtagaataatttcaagaagaaagactcatctaacatatttgagtattttcatgcaagggcagagtatgtttcttcctttattattctttgctctcctaaagtaaagatttataagtggtttataatccttttttactttatattaagtttatctctagttttttatagtttttgctattattatggctataattgtctttaattgctatttttaattgaatgttaatgtgttaaaggaaaaccattaatttttgtatactgatttgtgtcctgttaagttcataaactagattactacttctaaaagttttatttgattgttttgacctttaggtatagaaaatcacattgcttgcaccttactgcaagttcacaaatcctttccattacttatactttgtaattttttatctgtatttaaattaagtagtccagtgcagtcaatattgaataaatggtactagttatagcaggccagttttacttttattatgaattcttacagtattatcaaagacgttctttatcaagtgaggcagtttttcctatttgcagtttgtcaagagtacatttttagttttactataaatttgcgttgagtattatcaaatgacttctttcatatagtgtcattatcatgtattttctcctttcacattatcaagtagagaattacatcaacagttgtcctaattcatatcatcctgaacaaattctacttaatcatggcacactaaatatgtaattttttataatagtttggaatctgaatttatatttttgaataaaatttgtcgatagtttttatttttctatatgtgattaatttgcatttttggaacaatgattttgccaggttcatagactgagtgagaaaggtccattaaaactagattcatgtattatttgctgattaataaattaaataacttagaagttatctgttaataaataggttactaaaacttgccagtgaaattagagctaaatttttatttttgtgtcaagtatttgccgaaattcactttctaaattgttattgaactatttaaattttctacctattcttgaatcaaattttgtaatgtatatttttgtcagtcttacatgttgttataatttttaatattatttcatgcatttgcataaaatgtcacaaatttctaaaatacgttatgttcctgtaattatgctcattatttcatactaacaattttcattctagtactcttttttcccctttcatcagacttaacaaaagagttgtctattttatgaatctttgcaaataagtagcccttgatttttatttttaagcctattttttatatggaattgtaattggaagatttaaaaagtcaatattgcactggaaaatataaaataaaaattaatgtttaattctatgtgt

SEQ ID NO: 54 - DNA, 1920 bp, Homo sapiens
ttctcttttctccacatcctcaccaacatgttattttttgtctgtttaataatagccattctaactgatgtatgattatatcttattttggttttaatttgcatttttctgattagtaatgttgagcatttttaatatgcctctgggctatttatatttcttcttttaaaaatgtctattcatgttctttgccgactttctaatggatgatggaatgctaaaggcccagacttaaccactatgcaatatagccatgtaacaaaagtgtacttgtacctcttaaatttatgcaaataaaaactcaaaaaaaaaaaaacaaaaaaacctaagatgactaaatgtcagaaaaccaggttttacatgccacttcatttgctgaaatacaacgtacacagcctgttaaaatgaagtcgtctgccccccaaaatatataatattaataaggtctctacctagaatcaccagtttacaataaatgcagaggatagatgcacatgttagaaaacaccataaaggtgaaatcacccaaagtctactcgacaaattatccaacttcttcaaccattaaatagcataaaagttaggggaggggaatctgttacagaacaaaggagataataatatatcatgcaaacacaaacccatctatatcttgattcaaatataaattgcaaaaaacggcttgaaattactatagaaatttcaacagaaacaaggtcttagataaacagtccccaacttttttggtaccagggaccagttttgtgggagacaatttgtccacagacaaagggtggagaggtggggatggcttcaggatgaaactgttccaccttaaatcatcaggcattagttagattctcagaaggagtacacaacacaaatccctcacatgtgcagttcacaatagagttcatgctcctacgagaatctaatgctgctgcccatctgacaggaggtagagctcaggcggtaatgcttgcttgcctgccactcacctcttgctgtgtggctccgttcataacaggccacagactggaacccatctgcagccccagggttggggacccctgtcctagataacatttagtaaatacagttaattcttttcagtgcaataactttgttgagattgttttttaactggctagccatatacagaaaacagaaactggaccccttccttacaccctatacaaaaattaactaataaaaaaacactgacatgtaagacctaaaaccataaaaaccctagaagaaaacctgggccataccattcaggacataggaaaggacaaaggcttcatgacaaaaacaccaaaagcaaaggcaacaaaagccaacattgacaaatgaaatctaattaaactaaagagattctgcacagcaaaagaaactatcatcagagtgaacaggcaacctacagaatgggagataatttttgccatatattcttctgacaaagggctaatctttgtcagaatctacaaggaacttaaaaaaatttacaagaaaaaagcaaccccatcaataagtgggcaaaggatgtgaacagacacttctcaaaagaagacatttatgcagccaacaaacaaatgaaaaaaagctcatcatcacaggtcattagaaaaatgcaaatcaaaactacaatgagataccatctcacaccagttagaacggtgatcattaaaaactcaggaaacaacagatgctggagaggacgtggagaaataggaacgcttttacactgctggtgggagtgtacattagtacaaccattttggaagacagtgtggcaattcctcaagcatctagaaacagaaatacaatttgacccagccatcccattactgggtatatacccaaaggattataaaccattctactataaaggcacatgcacacgtatgtttattgtgg

SEQ ID NO: 55 - DNA, 2520 bp, Homo sapiens
tttcagcctcccttctaccccactccaggtacttctgcctctgtggaattcctgctgattctaagccatgatgagcatggctaccctaccctctgatcttccctcctaccgtgctgggctcctgtaggaggggatccctctcttcctcctccaccaaatgttgtctctttttggaaccttgtctgagcactctccccaggtgggatgagtcacttcctccctttgttcccaggcccctttgttcctgtttcccctgagaggtctctgtcttcttcaccatgctgggagtaacctgaggacaaggtcaaggccgatgatgtctatgagcccaagagagggtctggtgcgtaaaagctgtttgagagagtatgcagaaggaatggacaaatgaaaattagagactgacttacaactggggaaacttctcgttgaccctttctgttcctaaagagagtgtcaccggataggggtcaggagcctgggctttcagttgcaacaagaagacttctttgctgtgggctttctgaaagacagttcctctctctgtgactcttcaaaacagacatgacaatcatgtgtgccctgcttgcccctgaggctgcgttgagagatataaaaccatcaggaaagtgctcagtggctgtgcacctgcagccagcacctctggccagtgttggagagcaaggaagggaaagccaagggaagccaattcctgggagcttctcctgtctgggatgccaaggtggaaatgaacttgagacccagaccaaacttgaggctctttcatagtcaggtaatttgggcacccagggcattgagatcagtctgccattcaccctgtggctagccacacctaccttcagctttttgacactggtacagggatcgttggagaagctctcggtgtctgaaatctcaatgtcctcgccatacagaactccagtcaggtcattccgcacctgtcagcaaagagaaagcagagggtgggtgtgctggggaccacaggaagggccagttccgaggggtcaccctggggaagtcaattgggcaaagcgattttctctaccgacaatgcaaagtgagtggttttgttttacattattaactagaccgccccacaaaaacttgaggatcccccagtcccaccctgcaactgacacatggatacaaggaggccagacagggaagggactttccaagattgcccagggagttcctgcaagagtcaagattagcacctttgctggtgtttctccaccacatcacactgtctccaaatcaggctattcaattgtgtctttgttaatattttgcactatttatttgcaacattatttcacttttatggtgaggaaatagctaagatattcaaagacaatatagagtaaaggaaagaggaaagaagtatggaacctgcctatgatgttacacgtaactatgtgtctactgacactcagaatgaggaatatctatggatgtgaaaagcaaagggctgcaaactccagtgttaccaggaccagccagtgtgtgaactagcctgtgtggaaggatattacagaatgatagggctgggcgtgggctcacacctgtaatcccagagctttgggaggccaagacggggggcttgcttgagcccaggagttcaagactgcagtgagccgtgatcacgccaccgtactccagcctgggtgacagaacaagaccttgtctcaacagaacaaaacaaaacaaaagacagtaatagttggttgctgagttagaatgtgggtcaagggttaccagaccttctgatttttgaggggagaaatcagaaatttagacttttaaaatataaaatcccctaatttttaaatgttgatactattttgaattaaaaaaaaaagaaagtaaggatcaaaccaaataaacttaaagtctgtatctggcctgtggccatggctgtgcaccctctgacatatagcaatggaaactggattttgggttttagtagcaagaactaggctggggttaggggatccagcttccaggtcctgctctatcactgacttgcttgtgacctcaatctctcgtgtgattcttcccttctctgggcctcagtttcttccgctttaggagatgcttaaagcacttcttgttcacacccattagcatggctattaccaaaaagcaaaaccacaagtgttggtgaagatgtggagaaactggaacccttgtgtgttgctggtggaaatgtaaaatgctgccactgctgtggaaaacagtatagcagcccctcaaaaaaataaatataaaattaccacatgatccagcaattccacttctgggcacgtacccaaaagaattggaagcagggacttgacagacattttacacccatgttcataagaacatttgttcactgcagctaaaaggcagaagcagcccaaacgttcactgctcgatgaatggataaatgaattgtggtgtatacagacagtgaaatactattcagccttcaaaaggaataaaattctgacacat

SEQ ID NO: 56 - DNA, 2640 bp, Homo sapiens
acatatgattttttcaaattttctaaatagtaattatttcctagctctgccttctgaaaagtcctagaattacaacaagctggaaacaatgaacaaatggagccttcagactgtaatctctaaatatgattttccttttagtgaaaagatttctttggagaaatagttgattatagatctaggtcaagacatttatgagatgaccctgggacattttatttttgtcagaaagcctggaaagtattaatgtgtctacacaaaacaaaggaaccaacttaaaagagcagtcactgaccacagcagagataattgaagcatcaaactgaataaaaaatataactcattcaagcagatgcaattagttatcctgaaaatgataaatgcatgaatgtaatcaaacattaatacttggtttcctgtgcaattccttttcagggtaatgaaaactgatgagtgagagttaaatgaaggactccagaaagaatgacagttacaatataatgatttgtgccccccaaataaaataattgatctacacaaaatacatcagtattaggcaaaactagatggtaaaaaaatttcgaagaaaaatagattacggagaacaaatcagaactcactgatcaaacttgatatgactaatttatagttatttgagaaatccacatgctgttttccatagaggttgactattttacattccaactaataatgtataaggcattctcttttctccacatcctcaccaacatgttattttttgtctgtttaataatagccattctaactgatgtatgattatatcttattttggttttaatttgcatttttctgattagtaatgttgagcatttttaatatgcctctgggctatttatatttcttcttttaaaaatgtctattcatgttctttgccgactttctaatggatgatggaatgctaaaggcccagacttaaccactatgcaatatagccatgtaacaaaagtgtacttgtacctcttaaatttatgcaaataaaaactcaaaaaaaaaaaaacaaaaaaacctaagatgactaaatgtcagaaaaccaggttttacatgccacttcatttgctgaaatacaacgtacacagcctgttaaaatgaagtcgtctgccccccaaaatatataatattaataaggtctctacctagaatcaccagtttacaataaatgcagaggatagatgcacatgttagaaaacaccataaaggtgaaatcacccaaagtctactcgacaaattatccaacttcttcaaccattaaatagcataaaagttaggggaggggaatctgttacagaacaaaggagataataatatatcatgcaaacacaaacccatctatatcttgattcaaatatcaattgcaaaaaacggcttgaaattactatagaaatttgaacagaaacaaggtcttagataaacagtccccaacttttttggtaccagggaccagttttgtgggagacaatttgtccacagacaaagggtggagaggtggggatggcttcaggatgaaactgttccaccttaaatcatcaggcattagttagattctcagaaggagtacacaacacaaatccctcacatgtgcagttcacaatagagttcatgctcctacgagaatctaatgctgctgcccatctgacaggaggtagagctcaggcggtaatgcttgcttgcctgccactcacctcttgctgtgtggctccgttcataacaggccacagactggaacccatctgcagccccagggttggggacccctgtcctagataacatttagtaaatacagttaattcttttcagtgcaataactttgttgagattgttttttaactggctagccatatacagaaaacagaaactggaccccttccttacaccctatacaaaaattaactaataaaaaaacactgacatgtaagacctaaaaccataaaaaccctagaagaaaacctgggccataccattcaggacataggaaaggacaaaggcttcatgacaaaaacaccaaaagcaaaggcaacaaaagccaacattgacaaatgaaatctaattaaactaaagagattctgcacagcaaaagaaactatcatcagagtgaacaggcaacctacagaatgggagataatttttgccatatattcttctgacaaagggctaatatccagaatctacaaggaacttaaaaaaatttacaagaaaaaagcaaccccatcaataagtgggcaaaggatgtgaacagacacttctcaaaagaagacatttatgcagccaacaaacaaatgaaaaaaagctcatcatcacaggtcattagaaaaatgcaaatcaaaactacaatgagataccatctcacaccagttagaacggtgatcattaaaaactcaggaaacaacagatgctggagaggacgtggagaaataggaacgcttttacactgctggtgggagtgtacattagtacaaccattttggaagacagtgtggcaattcctcaagcatctagaagcagaaatacaatttgacccagccatcccattactgggtatatacccaaaggattataaaccattctactataaaggcacatgcacacgtatgtttattgtgg

SEQ ID NO: 57 - DNA, 2340 bp, Homo sapiens
aggcctcagtgtcctagactagcacagaacaagcagatgaaacaaagtttatatcagaatgtcaactgaaaaagtatcatttaccataaatggaagataattgtattaaattctagttagatgctaaaacttcaagaacttttagagtctgtacctgcattctgttaaaaatatagattaaaaaatgctaacatgttaacacaaaggactttccagaaagacttaaagaaaagtgaaaggggaataactgtcttgcaatgtaattcattgtcgtttaagactgggtctatggaacaccctaaatcacctggttccatcacgttctttttaacatggagatggatagtttttccccatactctatatattgagcattctatagttcatgatttttctgcatagagaattgttcaagccgggggtgcaggctcaccgactggatagtgaatcaagaaaatagtgtgttcattagttcatcattaccctgagtttccaacaagaatttagtacaggaaagtagacagcggagctgggagccatctatttgaaactgtcttaagcaaaactaagaaaccgagtaagcttgcttttggtgtctttcatcccttcttgtgtgccccctaattattcactccccaatgcccagacattatgatgccttctcctgctcagagacctttctgggaggaagacctactcagacctggtattccctcatcctaggctctaccctatttttcatccagctgttaaagctgagtgactaatttcacacttatgtacgaatgacccataactggcttaatgctgtgaccatcttgggggtattcaaagctgataaacacttttttaagttatataataatcaaagaagcttatctttctgctttatttcaaatttcaccccacaggccttacttatttttaagatcaatgattttgatgggcccccccttcccactcttaattcagggtatttctggccccatccggatccaaactctaatgctcatctcttccatactgtcctttgcaggtcatcggtattgcaagagttgcataaggcccaattcagtctctgccccaaaagctcaagtccaaacttcagaatctgggaggacaaggattcaggaaattttgtcagaactatgagctttgaactttcacttttatggtgagggtcacatttggtctgaatcaattaatccattacccgcccccccccccccccccaccaccaccatgtgtgaattcaaaataatcaacttgggtttattataaaaaacaaaatatattaatataagtatactaagatttttctagaaaacttggccgggcgcggtggctcacgcctgtaatcccagcactttgggagaccgaggagggcggatcacaaggtcaggagatcgagaccatcctggctaacacggtgaaaccccatctgtactaaaaatacaaaaaattagccgggcgtggtggcgggcgcctgtagtcccagctactcgggaggctgaggcgggagaatggcgtgaacccgggaggcggagcttgcagtgagcccagatcgcgccactgcactctgcctgggtaacagagtgagaccctgtctcaaaaaacaacaaacaaataaacttagaagaatatatgtgactattggccgggcgcggtggctcacgcctgtaatcccagccctttgggaggccgaggcgggcagatcacgaggtcaggagatagagaccatcctggctaacatggtgaaaccctgtctctactaaaaataaaaaaataaaaaataaaaaatgcgaggtggcgggcgcctgtagtcccagctattcaggaggctgaagcaggagaatggcgtgaacccgggaggcggagcttgcagtgagccgagatcgcgccactgcactccagcctgggcaacagagtgagactccgtctcaaaaaaaaaaaaaaaagaagaagaagaaagaaaaagaaaagaaaaaggaaaaagaaaacttaattctggcaatggactgtttctaaaataatatattaatactacttaatgaggaagaaaaaacctctgacatcctaaaatgccaagtgtttgcctttaccaaggtttaagcacacataaacacgcatattcaaataccacccaaagtggaggtgcaaagatcagcctgtaccgcacagtaacacagactgggttgttttttgtaaagaaggcaactagtccagtgagtaatcccttcattttccacacacatacccttctgttttctccctctcctccccccacaccctccactgcagttaaaacgtaattcgaagaagcctaaggtaaaagcccct

SEQ ID NO: 58 - DNA, 2100 bp, Homo sapiens
gtggacagggaaatcttaccttcctgcctctctatgttcaggctgagtgggtcagaaggagagtgtattaggtaagaaaatttatcagtattatttagtgaacactggatttatccttttgcattctggctgtagtacccaacttccacatggcaatgcaccctcacctcagccctccgcccacgtggtccccttgctgagcactttaatgaatgactgcatctcattttcacagctatttgatgcacctgctattattactcttattaccattttccagtgggaagctgcttcttgggcagggtggatttccatctgcgtctccttttcggtgttgaaagctggtaagtgaggacaccaggattggaacctgggtagtctgagtccagaatctctattttcaagtcttcctgctctctgcttctggcaagtttgatgtccacttttgatcttcacctacattccagcataatagctacttttggttgttttctcagcagcacaagagaagtgtggcgagatttttaggtgagtcatctagagaagttaatcttattttgggaattctactggcagcttcaggtggggaaaattttgttattttctatcctcctctaggttctaaaagggaagaaagatggtgagcgtagaaagatgtgactgtattcactattcaccctttgtcgggtggtgagtaagcagcttgcaaaagcaatgaagtttggaacaatccagagaaccaaactttcagctgccagagatggcacctggtatcctgggtacatctgcctgtagggcccagaaagagctggaagccaagtgcatggatcaggtctgtaggaaggtgggagagccaggaatcgagtgtcagggggcatttattacccatggaagcaggtttttgtcaattttgttcactgctggatcactaacacctggactggtgcctggccaggtggtggcttcataatcatttgttgagtgaatcaatgaatgaatgaatgaacagctgtagcagatgctagcagggcttcctatttcttccatcaccataaaggtgaaagacatcataaacgggaatttagacaatcctcagaaattttcaactgccatgtatcttgacttgatgcttctagtagttatatttatttgtaattcaatctttctttttaaatagttgaccaagtgtggtggctcacgtagtcccagcactttgggaggctgaggcaggaggatcacttgagcccaggaatttgagaccagcttgggcaacatagtgagacctcatctcttaaaaaaaaaaattagctgggtgtggtagtgcacacctgtggtcccagctactttagaggctgaggtagaggattgcttgagcctgggaagttggggctgtagtgagctttgattgcatcactgcactccagcctgggtgacagagcaagaccctgtctctaaaaaattaaataaataataaaaaaattaaaaagtaactcccttttctttattttcaggcttccttcccacctgctaattcaaacactttacaaccaaaaatatcttaccttgatcctgtttctttctctataacctctctatttctgtttctttcaaccaaatttcttaggtcatctataattttgtttctactttttctatgcatgcctcaatccattgccaactcctcaacctgccccaagtgcccacaactccaccaaaagtaattctaacattttaccaatccaatacatcacagttttttataaaaaacttaagaaatatactttagttgaatttgaaagagttgcccacttgtgttaaatatttctttccttgtgtctgggatatcatttgattctgattctattcctaattctctgaccaccctttcttcgtagatttctcttcctttgttcagcctttcacatccttggagttccatcctctggtgattgtttgtcctgttccatacattctccttatatgagcattgtgttttagcttatgaatggtcacatgacctagccaggccaatcagagtcttccatgagacttttgtttatttatttatttattaatttatttattcttccacatgccatc

SEQ ID NO: 59 - DNA, 2100 bp, Homo sapiens
gcacacctgagcaagggagaggaaagggttcttattcctgacacaggtagcccctactgttgtgttgttcccctgttggctagggctggaacgcacagtcaaagctaattccgattggctattttaaagagagcaggcgtaggagccagaatggtggggcgagtagtttggcgggaaggtcagttacagaacaggtgactcaggatgactcaggtcagagcaggtgaccaggggtgtctcaggatggagcaggtgaccaggggtgacacaggatggagcaggtgatagaggctaggagggggttgtttactgaaactaggggcaaggagatgacgagaacgagaaagttaaactttaaaatgaagaacaaagaacaggggagctgaacatactgatagaactctttcaagtctacttaggtaactatttgtttgtttttctgcttctaaaattttgttgaaattttctcctttcttattctcattgttcttgaggtttcgtgtatttaaaaaatcttcttactgtaattgtcatagttgagtagggagcaacgttagattaatatattcaatacttcactgttacctggaataagagccctctctttaaacaaaatattatgcagaaatctaatacaggaagcaaataaaaactagaactactctggttcaaatagagtgaagacagagcagatcttgttcttgtaattgaaaggaatatgatataataagtattgacaatattttcttctcaccaaataagtttctaattctatatataaaggaaatactttcagaataaaacgaatatatgagttttatttttaaatcacaaaacgaagttcaagaacatttttgaaactgggaagattcatattttagtatctgtcaaatgatgataaattcggaagccagtgtaatttataccctaggggctgaggtctaattcaacatattccagtttctattttctaaagctaaagaaacatgtgttacaatgtagatagggaatactttcttaatgaaccatgctgaactgtaagatttttaagactcctttttaatgcattacattacactgtatcttgttttcacatttatggtgaggttaatataaaagagacattaaacaaatatatttctgctctttacaaaggatgattattgttttcttacatttcaactaaaaatttctataatattatacttgcaagaagtataacactcttaatgagcaatacagttaaccttaaggttaacttgcaaaatttcatgtctaatttagtatcattaacacattgaaaaatctctcctaaatttcactcatcttgatcaaaatccatgttaaaggttttgaaactacacttaatacatctgccttattttatgcccccacttatactactagttattaatgcactttggacagctggttcctctgccttttgaggattctgtggtgtaactgattggttctcagctttttccactgcccatttgggatgcaaccctttcaagtctgcttaggtaactataatttgttcatgtgtttttctacttctaaaatttggctgacattttctccttattcttgttgttcttgaggttttatgcacttaaaaaatctttctactgtaattatcgtagttgagtagggagcaacattagattcatgtattcaatacttcactgttacctggaataagagccttttttaagggctcttactgaaaaacacaatacacttatgttcttctataatgttttaaggaattttttaacattaatctcctgtctcagcctttaaggccattaaatgacttaagatagttgctgtggctccaaacattgtatccacatttccacaagaggaagtaaaaaaagaaaaaaatgagtcttgccctttccttgtaaagacaatttccacaaggttcacattcctctggtcagaaatcagtcatacacccacgcctagctggaaggtaggttgggtaatgtggactttaattcagacagtattgtgtcagtaaaagtcaggggttctattattaaagaagcagtgtaaacagacatgagaaaacaaactgtagcctctgtgctt

SEQ ID NO: 60 - DNA, 2160 bp, Homo sapiens
taaatataaataaatcatcactaggtatgttctaataaaatttcagaacaccaaagatgaagataaattttaaaagtagccagaggaaaaaggtgaattacctttaaaaggttacagtttaacaagaagtctgacttttcaactacaaagattttcagaataatatcttcagtatgctgaatgaagttttaaaaagcagctgccaaaagagttttaaactgtgtaggtatatttcaagactaaagggaaatgaaaacatctgtagataatgaaatctgtaattaccaactgaacttaaggatatgcttcaagtggaagaaaagttaattgaaatggagggtctgagatgtaagacagaatgagaaataaataaaatagcaaatatataaataaatctagatgaacactgactgcaaaaagctacaataatgataacatctttgggcatttaaaaagataattaaaataagtaagaggagtgacaaattctttgggaggtgattaaattgaatgaaagtattctaaggtacttgcacagtcacaaggggtgaaaaaaggttttgtttatagtaagactttgtcaagtaggcatatgtaatttccaggttacccataaaaaattcaaaacagaaagtgaaatttccaaattagtagagaaaaaagtgacataataaaaattattcaattcaaaatgaggcaaaaaagaagagacaaagaaacacgggatatttagtacaaataaaaatcacaacataatacagttcattaaaactcaaatttgttagtaattccattgattatatatgaattatgtgttccatttatgaagcaaaatatcagaagaaaaatccacccatatactgttttaaagaaacatttaagacataagaatacagaaagtttggcagtaaatgaatcaaaaatgaaataatgagtctacgtgaggataatcaatgttcactgaaatattagggtgaaatgctgatggagaactttataatagatataccaggtctataacacctgagcctaatcaaatgtaacaccataaaagtgaaatatccagacgctatccaccagattataggaaatgcaaagcaaaagaatattaaaagacataaagatgactcaatcttgcaattccagaacatgggaacttctaaaagataaattgttttaatcaatatgtagtaaaaagggaaagggaactgttattgaataaaagtgacatcgtgaccaaatgtaatgtaataactttggacactgcttgaagaaaccaactataaaaattcatattgagtcagtcaagaacatgtttatattgactggaattttattactttaaggattagtattaatttttcagtgtagtaatggattgtagttatagtaaaaaaaaagttcttatttttgaaatttacattgaattattgatgaataaaattatgtgatatttggaattttctttaacataattttcattattaataataaaatcatgaaaaggaacaactcttgttgaatgcacattggaactctgttgaagcaggcatttctgacctagggggaaaaaaaacataaaagagaagatttttatgtgataaatacaggtggttgccaggggctgccgggtggggaaaatggggagatgttagtcaaatggtacaaagtttcagttgtgcaggatgagtaaataagctctggagatccagtgtacaacatgatgactatagttaataatactgtattatatacttaaaattttctgagtaggtttgaaacgttctcgccatacacacacagaaaagggtaactgtgaggtgatgaatacgtattcaagctaatcacgtaattagctcgattgtggtatttatttcacaatgtataagtaaattaataaatcacattgtactcaactatatatattttttgtcaattatacctcaataaagctggggaaaatgtaaaaataaataaataaattaccgaaaaaacccaaacatccataaatgaaaatgataccaaatctggcgccactttttacaatggatgtaaaagtcaagagttaaaatctttaacatgcatgcttactatgtcgaaagatcacgtacatgaaaacaaacatactttattgtgatttttttgaatgtaagcgatgaa

SEQ ID NO: 61 - DNA, 2219 bp, Homo sapiens
tataattattaactgaagtcatagtttacattaaggcttacactttgtgttgaatagttctatggatgagggaaggggctaaaatgcataattttatgcattcaccattaaaatatcatggggaatagttttactgtcttaaaaatttccttcatttcaattatttgttcttctctccactctctaaagccctggaaaccacttatcattttattgtctctatatttctgtctttttcagagcgtcatgtagctggacttatacagcaagtagcctcttcagattggcttctttaacttagtaatattcatgtaacattgctccatgtgttttcgtggcttaataggtcattccttttcattactgatcattttattctgtgcatgtaccacaatttgttcgtctactactgaatgatgtcttgattgtttcggttgttggtgattatgaataaacttgctataaacatttacttgtgtggatgtaagttttcaacttattcagataatatttaaaagagcaattgctgtatagtatggtaagattatgtttagccttgtatggaactgccaaagtggctgtaccattttgtattcctaccagcaatgaatgaaaacacctgttgatctgcatccttaccactatatgatattgtcatatttcagattttaatccgtctaatagatgtgtagtggtagatagttgcttaattttcaattctcttatgacatacaatgtttaacatctttttatatgtatatttgctatctgtatatcctctttggtgaggtgtctgttcagatctttttcccattttaaattggattgttttcttatttttgagttttaagtgttctttttatattttaagtgcaagccctttatcagatatgtattttgtgcatattttcccactctgtggcttgtattttaattctcttaataatatctttgcagaagtttttaatttgaacaaatttcacttttatggtgtgcttaagaagttgtatctaaaaacacaaggtcacctatattttctcctgttacagaagttttagactgtgggttttttatttagctctatgatccattttgagctaatttttgtgaactgtgtaaagtctatgtctggattctttttttttccaatgtagatatccagttgttccagcatcacttgttgaaaagattatcttttctacagtgaattgcatttgtttctttgtctaagatcagtttactatatttgtgtgggtctatttctaggctctctattctgttctattggtctatgtgttaattcttccataacatgctgttttgactattgcagctttatagtaaattttccatttgaattgtgtcatattcttctttgttcttcttcttgtgtattatgttgcctattctgagtctttttgtattttaatataaactttcttgtcaatttgttgatacacagaaaataacttgcttggattttaatgggagttgcaatgaatgtggaaattaagttgagaagaattgacatcttagcaatatagagtctttcctgtccctatacatagaatatctatctagatcttctttgatctccttcatcagacttttgtagttttagccacatagatcctgtacatattttgtttgatctatacttaaatattttatgtaatcaattgacttttgtatattaactttttatcctacaaccttgctataacagcttattagtttcagggactttttgccaactatgggattttctgcatataaatcatgcaaaatatgcaatcatgtcatcaccaaacaaatatagttctatctatgccttcccaatatgtacaccctttatttctttttcttgtcttattgcattggccaggccttccagtacaatgttgaaaaggaatggtgagatacaatattcttgcctcttttttcattttacgaggaaagcattccttttaatagtaggcagtcagaatataatatgtaatatttttaaaggcaataaatagacatctaagtgagttattttaaaattgagagtttaaaatcaaataaaactaaaagaatttaatagtttgcctctgtatcatggaatgaaggagttaacaagagctgatgagagaatctgctatttgtcactatatcttttattagcatttgacttttaaaatatgttacaatgaatattttaataattttcttcataa

SEQ ID NO: 62 - DNA, 2160 bp, Homo sapiens
acatctgtctgttttgtgtggccgtcacaaaataatcaaagactaagtaatttataaagaacaaaaatttatttctcatagttaatggaggctgagaagtccaagacgaaagtgctggcatctcatgagggtcttcttcctgtgtccccacagggcataagagtgtaagactatgaactcatttctgcaagccctttatacaatgatgttaattcattcatgagagtggggacctcgtgacctaaacacctcccattagattctacctcccaacactgttgcactgtgcattgagtttctaacacataaattttggggggcacattcaaaccataacatggagttttctgcattgagaaatgaaggatccatttaatacagggacctcaaaatacaaagagaaaactgactggctgtatggagctagacgaagaggagtaagaaactactatttgcaaggctgtgtaattccaaggactgttattcttggatgctatgatgtttttaaagaacaaactactatacatttgtaagttattaaattattaatattatttgagaatttcaaatggcttaactaatcaatgtgacatagtgggaaaattgggcctttaattgaagacacaatttgctgattaccacttggtaacttagcccttgcttctctaacccttagttcatcttgtaaaatgcattagttctactccataggggtattgtgagatttaaacgcagtgcagcatattaagcacccagtgtagtccctgatacatagtgaaacatcaataataaattgttgctactggtagaaatcccttggcgtttggtagatttccaataaatactaattcttctaaaacttttaatgattatgtagatagatatatgcctagatctggtaacaaatatgctatatcaatagtcaaaacattctctcttaattttattatgatatatattggaaatcttagtgtggttttgattatactaacataattatgtggcattagtatgccaaatgtactcacagttatgccaaaattacctgccccaaattacagctaatcctttctttggtcctaggagagatacgcacactagggatatcaccataaaagtgaagaaacactttattactggctgggcttgtttctgaaattctaacacagagttcttataacatggacttttccttgcctcctagttcaagcttgagggcttactgtgctcttgcagggaaagataaaagaaagtgtcagagtgaaagaatggtcaaatgtatgaactcttcttttatttattaatttaaatacagtgactctgttcactagtaaacacacctaacccctgccttagagtcagattaacatcttctttgaggacagcccaaagaagaaaaatgcaaggatgaagcctagagaggtttccatctcgtatacttatattccactatctttggttctttctttcaaccattagacttaaacccaactgtataattaatcaaacatgtggatatttccttggaggaagaaatagagaagtgtcagggaagttcgaccgccacctaagtgtgtctgcttttttaatgctgccttatggtctaaagagatgggtgaaaagcagagtattcatttcaaggccatactatattatatgccatctattcactccaggctgcttgttgtcaaggaagaataaaaaccttgatatcaaagagaattaagctctcaaaattagtttctctttcacataccaaagtaaccttgagctttctagcctgcaaatttctctcccttaatattctttctctgttccgttccactgaaagtgatgtcacagtggtgtagttagagtctgggttactctttcctgcagaactgttcttcagtacctctagatagaaaatagtccaacatcaagtcttgcatgagttttcctttaccaaagatctagtagtctagaagatatttaaaaatcacattattgagcccacatctgcaaaaaggagaagtatatacaatattcttaggactcaatacatattacttgtgtgcttgtctttgcatggacatgtatgtgttttaatttctcttgcgtaaacacttagggttggcattgctgacccacatggtaagtgtatgtttaactttataagca

SEQ ID NO: 63 - DNA, 2160 bp, Homo sapiens
tactgcttcatccttgagttctacaaaaacacacacacacaaaaaccaacaaaacttaactataggctgggtgaagtggctcatgcctgtaatcccagtgctgtgggaagatcttttgaagctaggaggtttaaaatcagcctgggcatcaaggcaagaccccatctctacaaaaaaaaaaaaaagccaggcatggtagtgcacacctgcagtcctagctactcagaaggctgaggtaggaggatcacttgaacccagcagtttgagattgcagtaagccatgatcacattactgcactccagcctggctgacagaacaaaacaccacctctaaaaataaaaatataaaataaataaaaaaatttaaaaacctaaacatagctgcactttactcaatatatttacagttctacatatgtaaaaacttgtatattgactatgttttaaatgtgtaggggaagtttctcacctaaaggagtcccatagtgaacatttaagagcaaatgattccttttttatttgtatttttggttttgcctctagcacatcaggtattctttaagaaggctatgcctctgaggttgcatgatcattaactaattcataatttcccttgcatatatttgggtattttggtgtttcagcctttcccacactttttttatttgcatgtcttcacgatcaccattatatctttgttccacctgtactattatttactcactctttgtctttaaatcaaatcacgtttcttactcaagtagatttagttttaagacaaaccttatggccgggcacagtggctcacacctgtaatcccagcactttgggaggccaaggcgggtggatcatgaggtcaggagtttgagaccagcctggccaacgtaatgaaaccccgtccctactaaaaatacaaaaaattagctgggcgtggtggcgggcacctgtaatcccagctacttgggaggctgaggcaggagaatcacttgaacccgggggggcagaggttgcagtgagccgagatcgtgccactgcactccagcccaggcaacaacgcgagactctgtctcaaaaaaagaaaaaaaggaactttatgtcgctaccataaatgtgaaattactagaactcacaataaatagaagttagtaaagacactgaattctaactagacgctattgcttgttgaaggctttgatcttaggaggattagaaagcattctaggccaggcacggtggcttcctgtgtgtaatcccagcagttggagaggctgaggcaggcgggttgcttgagctcaggaatttgagaccagcctgggcaacatggcaagaccctgtctctacaaaaacatacaaaacttagccaggcgtggtgatggccacgtatggtcccagctactcaggtggctgaggcaggaggattgatgaacctgggaggctaaggctctagtgagccatgatcacaccactgcactccagcctgggtgacagagccacaccctgtctcaaaggaaaaaaaaaaaaaaaaaagaattctagtggtgtggtgtggaagacacattctcagcagactaaggttgtatctttataaccacaaggattgaaaaagaacggaaggacaataactttctcataaggtgattcaatgttatttagtgctgtttctgtgtaccatcaaaaatcctcttactacacacagaatattataacaccatctcattgtccacatgagctcagaaattggtcatcaaagcagaaaagtctttaaaacattgatctccggccgggcgtggtggctcacacctgtaatcccagcactttgggaggctgaggcgggcggatcacaaggtcaagagatcgagaccatcctggccaacatggtgaaatcccatctctactaaaaatacaaaaattagctgggtgcagtggcagacgcctgtaatcccaagctactcgggaggctgaggcaggagaattgcttgaacccaggaggcagaagttgcagtgagccgagatcacgccactgcactccagcctgggcaacagagccagactccatctcgaaaaataaaataaaataaaacattgatctccaagaaagtagatcatatctgctctctatctgaccacattgttaaacttggttatgtttgcaggttaaag

SEQ ID NO: 64 - DNA, 2040 bp, Homo sapiens
caaagacaggccagtgctctactccttgcttcctgggctccccaaaagggagctgactcctcatccctcaacctggagaaccagttcagctcttcttttcaccagaatcctttccctgcttccgactcatcttctttttctcaaagctgttgtaactgtattgttctcacctgctttgcccaagacagattccccagtcctccccatcagtgtttggcatttattctgggtgttctactagtaatgcccagccccggtcctgggcttcctgctgtttctattgcatctccctaactcttacatccaccccaactcagtgtttttggccttcctcagcaaccaggaatctaaaccaccctccaccccatagcaccctatggatgacggagccttagttcttgatggtgatgcagacaccttgaggtgtggccatgacattcactcagcccttggcctggtagcagcaattttccctgataaggtccccaaactgaccctcagttgtcccctgcagtcccattagggcctgtggaatttacgacttccatacacagcaccaggaagttgaggatggctccacgtgctagctcagtctctttgccctctctctgcctgtggcagattgtattttccaaagatgactgcaccaaaatattccaccccatgttatcttcttaaatgtgaagttcacactaattcttcaagaaatggggcctctgtttacacctgctgaatcttggcaggcctataattatagtggttatgattctagtgatgctatatgacttctgagaccataaaaagacaatacagcttccacctggtcctattggaacagtcattcttggaaccaagccaccatgttgtgagaaaacccagcccacatgggaaggtcacatgtagggatgacagtccccactgagccccagccaatagccggcatcaactgcaagacatgtgagtaagcgaaccctcagatgattccagcccccagcctttgagctgccccaactgatgctttgtggaacagagaaaagctgtccccattgagctctgctcagatttcacatttatggtaaaaatctatatggtccttactttaagttactaaatttggggatgctttctttacatagcagtaggtaattagaacactgcctgatcaaactgcactgcaacttttactcggctgctaactatatggctatagccgagcatcatggggccaccgtgtctggcagtccccacatccgagttccaaatgcggagcacaaaagtcccactgtcactgatcttcccttccactctcagaatctcagtctagtatggggaagcaagggtcgaaccatgtgcttcccccgtcagggcagatggttctcttcctgcctggaaggaattccctctacataaaagcctctttccaccaggtatggtggctcaagcctgtaatcccagcactttgggaggtgaagtgggcagatcacctgaggtcaggagtttgagaccagcctggccaacatggtgaaaccccatctctactaaaaatataaaaattagctgggcatggtggtgggcgcctgtaattccagctactcgggaagctgaggcaggagaatcacttgaactcgggaggcggaggttgccgtgagccgagatcatgccactgcactccagcctgggcaacagagtgggactccatctcaaaaaagtaataataacaaaaaatttttaaaaagtgttcttcttcccaagaaagcagacatcagacatctttcccccttcattggggccttaattgcagatgggactctggaagagatactgacataagcataaaagtaggtccaagaatatttaacctctacataagaattcaaataagctattgaccttatgagagagtcacaatgatggacaccttcaaaggaaggagaagcaccatggagggcagaggagaaagaccatacctgcttgacttggtgtgagaggcattctagatgttattgacatcatataatagacagatgacaagcatagaag

SEQ ID NO: 65 - DNA, 2100 bp, Homo sapiens
tgtacatgcattcattttgctatcctgcatttgttctttcttcctcagaccttttcagtccctaagaaaatggtatcattctgggtaggacctaagatcagtgatgaaataacagaatgcaggatggcaagactctatatggagagggaaattttacagaatctaaacctggggatactaaattagatcaattgagtataggcaatatcagagggtaataaaatagtttaagaatacatagatgttttttgtttgttggttagtttttgcttttcttgtaattcaggttaaagatgtgacatttcctaacagcaccaaggaaggagcaccagagaacatgaaaaggcactcaagctaagtggtctgttgaactttgcatatcttcctctttgtcaatgaaagaaggcaaaaccaaacctaaatgaaacaaaacaaagaactacttacaaggagttttaaaatatctcaatctctgcgttacttatgtaacccacagagtatatagatgaccacttagaggtattcctactattaacaaaggtattagtatctgtgttttacccatgaaaaattagaaacttagaaaaatatcttgttcagtttcacactggtacaatatgagggtatgaaagttgtaactgactctaaagcttgagctattttcttatagatataatttaaataatgtctatcaactttcttgaaatattctcattgttacctaagaatttaaaatattgatatgcaattgatctaagagaggttaaacatgaattggaaattccttctccgatatcaggtttgatttttcacaaatatcaatgtttatgaaagtattcctaaaatttcaggtaaaaccacccaataatatgactgtgaaaaattatgcttcttcctctatgagctactacattcttaaattatctgagcattctattaaaacttaaaaaaatgcttaacttgagtctgcatgaatctgaattccctgcatatttaattttaaagaaaatagtttatttttttgttagaccaatactttacaaacttccccaaccaataagaaagaacaaagaggagaacatgaatatccctgggtatttgtgagtaaatccccaagagactaaccataaatgtgaaatttctttataattgtatgtcttcttctaaaatattcatgtggattgtgcattctattctatcttcttattttaataaaatctgttttaaattatttacttcctggaaacaaatctccctgttgtgttggtttatgaacatggttctattgccttcagtctattgtcggaaataaaaacagtcctgcagttgttgattgagtgtactatgcctttaagaagtcatggcactcatgcaacagccatgtagttgttgattgagagtactgtgtcttaaaaaaagaacttttgctaaataaactgactctgtgagcagcccttcatcatttaagtgagaaatgtattgaattaagttaccttgatattgccttttgttatatttttatttctttgatacaaaagagtaacaatttaattcgaaatttgaaatccctgaattgcctatcctctccagtaagtcactacacacctgtataggggagcagcctttcagaatatttttcctgaacatgagaatataaagcaggaggtggtcatatttgtttgagtagcacctcctgataccattaatctgagcagaagagtatgggtccatactagaacaggatatgactaggaaaatgaagaagaaatgaaagcaaagtattcaacagaaacatctatgctttttgccattagctgaatgtgacataagagtatagaatgatttgacacgattccaaatctaaatgtaaccaaggaactttaaatattattaatgagcatggcaaagtttagggtcagggggaacaaatttaaaaactatgagcattctcatgacatgagttaaaatgcaaagacgtaagttaaaacataactaatgacattaataaagtgattgaagctctatgtcacttaaagataagaaggtatgatagtttagacattgttctaaaggccaatctaagtgaaaaaagttttcagg

SEQ ID NO: 66 - DNA, 2100 bp, Homo sapiens
agttagaattctgtgaggtttgtataaaaggaatagagtgggggcccaaaaaccagtaagatgagaaagtactgtttgctcagttctaggatccatgaaataaataataaataaataaaaaagagaaagtagtgtttcctgccactttagaggaaggactcacatatcctaccttccatcagccttgaaggagatgagtgccctctctccaacacctggtggccttccctaccccttccccaaagcctccaagaaggcccctggcctagcctgatgcccactatcagcaggaacaggcacgacaaactttccccttcctatccctccccacctctggaaagggctggggacagcagatgtgtccttgttagttccatccatttcagctttggctggggagctaatttcactggagccaggataagcattagggtaagtaactatttttcctgtcttgggcagtttcctcactgacaaatgagggcagagttctaagctctcttctaattctaaaattctaatgtaaaaattgccagactagtggtggcgcaagcctgtaatcccagatactcaggaggcttaggcaggagaatcgcttgaacccaggaggcggaggttgcggtgagctgagatcgcgccattgtactccagcctggcaacaagagggaactccatctcaaaaaaaaaaaaaatcaccagactaatatttaccttgagtgttatgcgcatccatgtgaagagaccaccaaacaggctttgtgtgagcaatagttttttaatcacctggagtcagcaaaaggagatggggtggggcagttttataggatttgggtaggtagtggaaaaattacagttaacgtgcgttttctcttgtgggcaggggtgggggtaacaaggtgcttggtgaggagctcctgagactcattgtccaggagaaggaatgtcacaagatcaattgatcagttagggtggagcaggaacaaatcacaatggtggaatgtcatcagttaaggcaggaactggctatttcacgtttatggttcttcagttgcttcaggccatctggatgtatatgtgcaggtcacagggttatgatggcttagcttgggctcaggggcctgacattgaggattcttttttatcttcctctgatgctcttctataagaatgactctgttttggaagaaaacgcaattaagattttccatcacaacaaccactatctccaaatctgtattcattccttttaattcattataagtctcatctacctaatgagataacttttttgaagacaggaattgtatgctgtttaacagtgctttgtttcttccatagttcagtcatccttgatattttgcgggggactggttctaggatactgcccccacacaccagaatctgtgaacgctcaatcccttacatataatggtgcagtatttgaatataaccaacacacatccccccgccaccacccaattaacttttttactttttttcccccccgagacagagtcttgccctgtcgcccaggctggagtgcagtggcacgatctcggctcactgcaagctctgcctcctaggttcatgccattctcctgcctcagcctcccgagtagctgggattacaggtgcccgccaccacaccgggctagtttttttttttttttttttctttgtatttttagtagagatggggtttcaccatgttagcgggtggaccttgtgatccgcccaccttggcctcccaaagtgctgggattacaggtgtgagccaccatgcccagccaatttttgtatttttagtagagacggggtttcaccatgttggccaggctggtctcgaacacctgacatcaagtgatccgcccaccttggcctcccaaagtgctaggattacaggcatgagccaccgcacccagcctcagactaaactataataaaagagaaagcagagagagtaagagcacctcatatggaatcacctacatttcagaagctggaaagaaagagagtggtctacttgatgatatgaagcatgatcaatcagtatcaatactagctttagggtgaaggcatagccaaattggaaactgtgga

SEQ ID NO: 67 - DNA, 2100 bp, Homo sapiens
aaaaaaaaccttgtccaggcacagtggctcacatctgtaatcccaacactttgggaggccaaggtgggctgatcacttgaggccaggattttgaaaccagcctggccgacattgcaaaaccccacaaaaactagccgggtgtggtggcacacacctgtaattccagctacttgggaggccgaggcacaagaatcacctgaagctgggaggcaaaggttgcagtgaaccaagatcatcccactgcactccagcttgtgacacagtgagactgtctcaaaaaataataacgaaaataaaataatctaaaatttaaaaaaaccctaattcatagttatggaattattgagcatattaaataagataatgcatgcaaagtacttaacagcatcggacatattttaagcactcactgatgcttgctatatagttaaattatatagctatatgtatgtgtatatacatgcaaagatcaggagatatgctgacatagaatgactatggcagggtcctgaaagagaccacaaaagagagaagttttcacactggttctttccttttgggggatgctgacagttcccctagaaggcagcagactttgcctctgggagcaagccactggcctggcccagcactcctgagatgagcagaaatgggcagaggagagctcaagacagagcacaggccagactaatgtcttcctgagaggagagcagtgggaggaaaaagggggcagcaaagagacaagagattgcctccttcacctccaccaatgttacctaagctaaaacccctctgtctaccaaatcagccctggtcacaaactaaaacccaaacccaacaggaggcttacttatccacgccaatctgaatttctccatgacatggaccaggtgggactgtgggtttggtgccatgtacatgacctgtgacttagtggatggagttccttaggccacagcagcctctggctcaatgaagcttgatctactgagtacctggaccacatggggctctagcagcagtcctatcttgagcccagaacagtaactttcaatagaatatacactggtatgttgaatcagaagttcaaacgcccttcacctttatggtgacttttctctcaaggacctccactgctttcttctactatgccttcgtatcattgaccattccatcagtgagggccaccacagtccctcagaattctttaagactaactagggggagattagagtaccaatccttctaaacctttcaaaaggcttcttttgaacccttttcaaaagatttcttcacttagcaccctggaaccaaatggaagtgaatatttttgagaagacgtgacatctttctcctgggccttgcccagccaaaaatgttctgttatctgttgcaattaaaagagagcaaagagtaagaagtctctttccttaaagtttctttggccacttgagcggagcttcccagagcagtaaactcctttaggatagggactgttggaattaaatgagctggggaaccacaacctagaaactggacttcagctttgtaaactccgaaactcattatcactgtgatggttaatttgatgtgtcaacttgagtaggccatagggtgcccagattaaatgttgttctgggtgtgtccatgaggatgtttccagatgagaatagcatttgaattggtagactcggcaaagtagattgccctcctcagtgtggctgggcattgaaaaggccgaagaaagaatttgcccctcttttcctgcctcactattgagctgggatatctcatttcaccttctcctgccctcaaactgggatttacatcatcagcacccctggctctcaggcctttggacttggaccaaactgcatcactgactttcctccatctccagcttgcagacacagattattggacttctcaacctccataatcatgtgagccaactactcataataaataaataaataggctgggtgtggtggctcatgcctgtaatcccagcactttgggaggcccaggtgggcagatcacgaggtcaggagtttgagaccagcctggccaatatggtgaaacccccatctctactaaaaa

SEQ ID NO: 68 - DNA, 2040 bp, Homo sapiens
tacaggatgaaggtaacaataagagaaagtgatcataatagtcaatatttaatacatatttaatatatatatttttagatttaacaactggttaaatctattaaccatatatcatctaaccatttctataccttcctatcactcttcttttcctattctctcttaattccaattttcctacaacacacacactcatatacacatacgcacacatgcacccactatccataagaccatcacgtctggggattttgcacatacagagcctaataaaattcagcaacaaagatcattcaaattcataactcaaaaattatcaggtacagaaatacatgaactgaggtgaagaaaagccaaaatactgggagaagtagaatatctttcacaaaagaccttctaggaagatcatatggtactgttctgcaaatctttccttttacaacattgaatgttttaatgtgagctttgcagattcagtttcaaatcttatcactaatcctgcccttaagcaaagctgtaaagaaggtgaaattaattttatacatttcctattctgcattctgtcatcctcatcttcctttgagggtctaacaacttcctaccactttctgcttgtgcccattacaacccagattttcatcttttgtacctggaacaggcctggccttccacaatgctctatgtgctatgaaagtcagtttctgctattatcattgtgttctattatttcatgtattttctaatagccttaaagattactttgaataggcctggaatctctaacacaattaaatactatagcaggcatatagtattaccaaggaaagtaaagcaagatatagaacaaattgaatgatgaaaactgagacaaactggagacagggaccatgtccaaaatggacatccattattcaactgtagcctcatgttatcatgtgggaatgaaggcctagtgttgtaagattttagatttttcaagaggagccagaaatttgtaccttcatacaaaaatttcaatatttgtaaaacaccataaaagtgaaaaaaatacctacaaatcagaatttagactatgttgacattaacttgctacctctgtgataagcgattttagaataagattttattcttgcttaattcttctcttcagagatacctagtgtagtaggagaatctaacacttaaaaacaggtcaggtatggtgtctcacacctataatcccagcacttttggaggacaaggtgggtggattgcttgagcctaggagtttgagagcagcctgggcaacagaatgagaccctgtctctacaaaaaatacagaaattagtcagctgtagtggcctacgcctgtactcccagccatttaggatgctgaggtgggagaacacttgagcccaggaggtcaagtctgcagtgagccatgatcatgctactgcactccaggctgggcaagagagcaagatcctgtctcatataataataataatagtaataataataacaattttgtgatgggtgataaatatcataggggcaaaatgtcatgggagcacagagatgggagaggggactttcataagcctagaatgttctcaaggagtatgtctcagagaatatgaatctcaaatgaaagtaggcctttaccagagagagagaaaataaagagtattccagagagttgtgtgtgggaagatgcagacataagaaacagcagcatttacttggggaaaaaatagtttaggttctgttcccagataagtggaattatatcagatacagtttttaaggagagtctatttggggcaggagggcctcttgagttcttattaatagttttaaaatgtgaacacaccttactgcacattaagcacatgtaccccagaacttaaagtataataaaaaaaaattttaaaaaaagaaaaaaaatgtgaacacacctctatttctctctccaggtaattttaacatgaccttgctactcccttggatgaaaggatcatcacgaagttttacaacaaactttatggttatggaagttct

SEQ ID NO: 69 - DNA, 2100 bp, Homo sapiens
tctaaggcttctggacctgaactgagccatgctaccagtatttcaggatgttcagcttgcagatagcctgtcgcggaacttctcagcctctagaatcacatgagtcaattcccctaataaatctccttttatctatctgaacatctctcttcatctctccatccatccactcatgtgtccatccatccatccatctattgctatctatctatccatccatgcatccatccattcaaccatccatccacccatccatccatccctgtgccatctatatctatctatctatatatctatctatccatgcatccatccatccatctatccatctatccatccatcactatctatccatgcatccacccatccacccatccatccatccatccatccatccatcactatctatccatccatgcatgcatgcatccatccatccatccatccatccatccatccatccatttatcgctatctatctatccatccatgtatccatccatccatctgttcatctatcactgtctatatatctatgtatctatctatccatccatccatgcatccatccatgcatccatgcatccatctatcactatccatccatccatccatccatccattcatccatctatctgtcttctacctacctacctatctaactctctggagaactctgactaataaactagctttataaacatgttattctctctctgcaatgtctattgctttatcttcaggaacattccacacatcctgtaagacttcagttaaattatctctctgtttcttctccaatcatcctctgccttccctagtctcctaacgtactttgtacatctgtcacaaacccctcatcatatttactgtaatttttttcctacagatttggataggaattgagccatttttttaatttcacttttatggttgttacaaataaaagagcaagcaggcccctcactgtaattcacctgtatttgcatttaacttattaaccaaggcatactatttcaaataatctaatatagtatttcctatttaataaccaaacatacagaacagttccaagcacatgtaaccatgtgatacattttcctctttgaataataaatatatttcttataattaatatgtgataaaattgcaatatttttaatctcctacatccttctcttttaatcaggtttccttatcaactggttcctatctcacggggttgttgcagagatgaggaaaaaaagtattctattggttcatgcatctcaaaataggcagattcttttctctgcttcttccttcattggctcaggtgtggagtgcttctcccaattatatgtgccagccttggtatgttctcattgctgtaccacactgcctgagacatccaagaccacatcttcctttgggggcacattggacctttgtcattggcactggcagggaagcttttatttcaccaggtctaaggcaattcttccaaaaaaatcccaaatagtgaaagaattgatttattcttctaatatttaagcaaatgtaaaaaaaaagttacattagttatgtttttttcagattttggatcagtgagacttcattaaaacactttgaggttataaagcaagtaatttttgtttccagaaaagttagtttcctttggctgaagggacatctctatgcaggccagatcaagacaaaaataacttttaagaagggaaatgagggaatggagtttggaaaacataaatcccacagcaaagtacgtcaccaacaataagagtcatctctttcacagaggcctttcctagaaaagccctgacagactaggagtccaatcttcggctcccatagcacccatgcctgcttccactctggagcttactactttgcgttgaaattaatttttacatgtctatggcttctattacaaatagcttattgaaaagagaaccatgtacattacaaatacttttttagagttgctgaactgaacaaatcagtacctacggggttagtatgctggcttctattccagcagggttttgagccatgagattttgaatgctcccgacattgttagttcaggatgattaaaaatat

SEQ ID NO: 70 - DNA, 2040 bp, Homo sapiens
tgagaaaatgcaagaaaggaagccagagacgttgtggactgcaggctccttccccatcatgtcactcaaacacaatgtttcatttgtaaaacatattttaaaagattatgaatgctatttaaaagaacactaaaggccgggcacagtgactcactcttgtaattccagcactttgggaggctgaggtgggcagattacttgagtttaggagtttgagaccagcctggccaacatggcaagaccctgtgtctactaaaaatacaaaaaacaaacaaacaaacaaacccaacatggtgatgtgtgcctgtggtccccactacttgagaggctgaagtgggaagatcacttgagcctagtaggtggaggttgcagtgagccaagatcacaccactgcactccagcctgggtgacagagcaagaatacatatatatatatatatatatatatatatatatatatatatatatatgtatatatatatatatatatatatatatgtatatatatatatatacagcaatcaaccaaacaacagcagcaaaacattaaagtgacatgctgactccttggaaaggtggtaagctctttaggcttcctaatcagagaaggcagataaaaagagtaatttaacatctttctaatctaccccaatggaatttgcagtgattttctttccattttttctcattgtttttcaacctgatcactaataggtagctgaaatggagtcacttaatggtttttgctttttaagaattccaaactcacagtttgaaaattatagttcttgcatttgaagtttttcttcatcgtgcttggcctgcctggtggctttttgttttgttttgttttggtttggtttggtttgtggtctctttcatctacccaaagccagttgaaataaatcaaaagttgtctcactcaggaattgtctaaagtaaataaatgaaaaaaaaaaaaaaaacaagactaataattaggcaactcattgagtaggctgttgaaccagctaaagtgggaaagaaattattcaagttctaaacctttctacttgcaaattagccaaaatcaattgcattttaagcactgcatcaccttgattgatttttaaaatggatagcactttgctgttcacatttatggtgagccgtgaaggacttggcaatgggcatcttttctaccgtgttctgcattaaactctttaaatagcttctgcttcttaatgttgaatgaactttactgcagactgagtctgagggtttttttttcaaggttgaaatacattcttcagactttactttttgcatgtagcattcttcttacttaaaatacctaagagtttctaactaatttctttcagccacaggaaatatttctgtaattttagggttaaaatggagaactataggatgaaaatataatacaagaaaatgaattaaaccctaaaatttattattatatgtgggtaaaagtaggggggaaaatcactggttttgaaattaaaagatgaaaattgtgaacacttgcatggagaccctatttgtaaacttggacaagcttcagtgccctctctctgcatcttctgtcattattatttttcctcaaggcatcgttttgaggattaaattagaaaatgtgaactagcttgagagtacagtcagcacttgaacagcatgggttggaactgcaagagtccacttatacacaaactgttttcaaacaagctctgatctaaaatacaatatttgtaggatctaaaacacgtgtatacagagggccaacttttcacatatgaaggttctgcagggccagctgcaggacttgaatgtgcctggatttgggtataaacaggtagtcctggaaccgatacaccaagtatactgagggacaattgtaataggcacaaaaatgtcgattgatagattttgttctttttcctgaaatgcaaaccaggacacttacaaaactaaatgaataattacttatacatttagtgtctctgtgcttctctcttttaccttttcctacttcttccgtat

SEQ ID NO: 71 - DNA, 2100 bp, Homo sapiens
aagtacaattggccaggcgtggtggctcatgcctttaatcccagtactttgggaggcgaaggtgggcggatcacttgaggtcaggagttcaagaccagcctggtcaacatggtgaaaccccgtctctactaaacatacaaaaattggctaggcgtggtggtggcacctgtaatccaaactactcgggaggctgaggcaggagaatggcttaaactcaggaggcggaagttgcagtgagctgagatcgcgccgctgcattctagcctgggcaacagagcaagactctgtctcaacaaaaaaaaaaaaaaaaaaaaaagtacaatcagtgttctgttgtgttttgttgttggttttttttttttttttttctttttttagacaggatcttgctctgttgcccaggctactatgcagtggcacaagaacagcttactgcagccttgacctcctgggctcaagtgatcctcccacctcagcatccataatagctgggactacaagtgcacaccaccacacccagccaattttttaatttttttgtagagacagggtcttactatgttgcccaggctggtctcgaactcctaggctcaagtgatcctcccacctcggcttcccaaagtactgagattacatgcatgagccaccatgcccagctctattgtgtttcttgttttttagcttgacaggagggtgtcagggatttcctggcttacagaaatgatatgaatttgcagaagaaactaaaactgttaacaaacatttgaaaaaagttagccccattatttattttttaaatgcaaattaaaacaacatacctcagatttaatgtaacttctatcaaaatctctgctggcttctttgtagaaattgacaaactgattctaaaattcacatggaaattcaagggaccaagaatagccaaaacaacttagaaaaggaacaaaattggagggcccacactttctgacttcaaaacttgctagaaagctacactaattaagactgtgtggtactggcataaagacagacaaacagatcaatggaataaaattgagagtccagaaataaaccttcacatttatggtgaattcatttttaagaagggtaccaagataattcaagtaggagaaaaatagtcttttcaacaaatagtgccaggacaactagatatccatataccaaaaagtgaagttggactcataccaagtacaaaaatcaactcaaagagaaaaaaaacctaaatgtaaaactataaaactcctagaaaaaaagatgtaaatcattgttactttggattaaacaatggtttcttttctttttttttttttttttgagacagagtcttgctctgtcacccaggccggagtgcagtgacacaatcttggttcacttcaacctttgcctcccaggttcaagtgattctcctgccttagcctcccaaatagctgggattacaggtgcctgccaccacgccaggttaatttttgtattttcagtagagatggggtttcaccatgttggcaaggctggtctagaactcctgacctcaggtgatctgcccaccttggcctcccaaagtgctgggattacaggcgtgagccactgcgcccaccctagacaatggtttcttaaggtacaaaaccaaaaagacaagttatgaaagaaaaaaaatagattggacatcatcacaattaaaaacttttgtgcttcaaaagtcatcttcaagaaagtgaagccaaaaacagaatggggggaaaattttgcaaatatatatctgagaaggacctaatatccaaaatgcataaagaactcttacaattcaataataaaagaaaatcaatccaattttaaaataggcaaaggatctgaatggacatttctccatagactatacacaaatggctaataagcatattaaaagatgctcaacatcattagccatcagagaaacacgagtcaaaaatcacttacgataccacttcacacctattagtacgactataataaaaaagacagttaacaacaagtgctagcaaggatatggagaaattagatcctttatatactgctggtgagaatgtagaatggtacagccct

SEQ ID NO: 72 - DNA, 2100 bp, Homo sapiens
tagaaatattgcaatggaaacttcagaagtaaaaatgattataacgggctattataaacaattatgtgcaatatttaatcaggaagaaatagaaagcttgaatggaccaataaaaattaagagattgaaatatgaattcaaaactttgcaacaaagaaaagcccaggacgagatggcttcatgaatgaattctactaaacattcaaagaagtattaccaatatttaaattctcccaacaaatagagatagaagaaatacctgcaaacacattttacaaggcaagcatcaccttgatccctaagccaatgacatcacaaaaaagaaaactataggccaatatctctgatgaacattgatggaaaaattctcaataaaatattagcaaacaaaattcaacatcacatcaaaaagattatacatcatgaccaataggatttatccctagcatgcaaggctggtttaacatacacgaatgaaacaatgtgacacatcacattaacaggatgaaagataaaaaacacagaattttctcaatcaacacagaaaaagcatttgacaaagttcagcatcctttcctgataaaaactcttaacagtttatgtatagaaagaaaatttctcaacataatataataaaggtgatttatgaaaaatccacagctaacataataatcagtgggaaacagttgaaagctttttcactaagatccagtgcaaagcacaaatgcccacttttgctacttctattccacataatattggaagtactagcaatagcaatcagaccagagaaagaaataaaaagcatttaagtcagaaagaagaaaaagtaaaattatctctatttgcagatgatataatcccttatgtagaaaaccctaaagattccacaaaaaactgacagaatgaattaattcagtaaacttgcaggatacaaaatcaacatacaaaaatcagtagcatttttatacactaataacaacatatctgaaaaagacgctttaaaatcccatttatgaaagcataaaaatagttagaaataaatttaaccataaaggtgaaatatttgtataccgataactataaacctttgataaaaaaagttgaagaagacacatataaatagaataatattctgtgttcatgaatcaaaaaatttaacaatgttaaaatgtctgtattaaccaaagcaatatacaaattcaatgcaatttctatcaaaatttcaaggatatgcatcacagaaatagaaaaaaaattcttgaaattcatatggaaccacagacacataaaaacagaataggcaaaggaacaatgagaaagcaaaacaaagcttgaggcatcacacttcctaagttaaaattatattgcaaagctacagtaatcaaaaacagtatacaaatggcatgaaaacgaaaatgtggaccaacggaacagaatatagagagccagaaacttaactaattttcaacaagggtaccaacaggacaccctgaagtaaagatagtttcttcaataaatgattctgggaaaattggattgcaacatgcagaagaatgaaattggaccctaatcttgcaccatatacaaaaatggactcaaaatagataggagacctaaatgtaagatgtgaaaccataaaactcctagagaagaacatagggggaaaaattccttgatattggccttggagatgatttttggatatcacaccaaaagcttaggctacagaatcgaaaataaataaatggaactacatcaaactacaaagtgtctgcacagtaaaggaatcaatcaaccaaataaaaaggcaacatacagactgggaaatatattttcacacagcatatctcctaagaggctaatattcaacatttgtaaagaacacttacaaatgagtaacagaaacaacaaacagcttgattaaaaacaggcaagggacctgaacatacttttctccaaaggagaaataatggctaacaggatatgaaaaggtatacaacattgctaatcattagggaaacacaaatgaaaaccactatgagatatcacccttcacccattaggatggctattataaaaaaaaaaaaagacaagagg

SEQ ID NO: 73 - DNA, 2100 bp, Homo sapiens
agtaagtggtggagccaagaattgaaccacaatgttcaggattcatagaatgattagaatatagtgaaaacaaagcaagagataaattagagagctggcaggcggggcccactcatgagagatgcatgtgtacgccatgtaggatacccaggttttcttcttttggtgaacggaatctcttgaagggtgttgagcaagacagcaatatgagcagagagttagaagccccaccactcaggaagcaatgcagaggtaggcacgggagtgcatctgagatgaagagaatgggctgaggttgcagctcagaggcataaactagaattgaatgtgctgggcctcggatggactctaagcctcttgttgagtgttagggagacagggccatcatggaagccagctttggacagcaggagggacaccttttctaccgagatgaaaaaggaatggcaagttgtggatgtgggggcatgggtgtggtttacatctcaggatctctatatgctctattaacctggagatgaggttttctgttataaattggaggaaaatggtgaagttaggagcttaagagtgagaaagtctgaaatatccattgtggagagtggaaacaggtacaactaggccttgattccctccttaactgcttcttcatcctcagctactcactgatccatcagttgtccagtttgtcatctgtgactttgatttctattggccctttccttttggaccttattggtcctttcctttcagcatatagaatgttcatgtctctctcaacttaaaacaacagcaaaacccagcccatgaccttctgcccggtaaagtcatcctggtagtgtccaccagcctggcagaacctgtcttccacagcagcctggtgacaacaagaagccacagcagcagtgtttggcagtggtctggccctctccagacaaaataagcatccctggggttcctggacaatgaagggcaaaggaaaagagaacaggtctcaagaatatactgacaaatctccaagaggaattcaagataatatggcaaatgggaattaaatgagaaagtaaaagagaggtgaccatatgccaatctggagcaggtggaatgtgtcatgatggttcaagggcaatcaagagttcacatttatggtgaggatttctcttgattttttttcctcctgtcatcttattttgtgttttcagtttttcatgtattgttaccattaccttatattgattgcttcttgtcttttgcaatacatcctaaatatagcaggttcttgaataacatcacctcgttcaacatcattttatgataatgttgatggggaaaaaaaaatggctccaggctggagccactgtctgcatgaagtttgtactttctctccatgtctgcgtgggttttctctggtttcttctcacatcccaaagctgtgcacatgaggtgagctggcatgtctatatggtcccagtgtgactgagtgtggatgtgtgagtgcaccctgcgatgggatggtgtcctgtccagggctggttcccaccttgtaccctgagctgctgggacaggctccagccacccatgaccttgaactggaataagcacattggaaaatgaatgaacaaatgaatacaaattaggataaaataaaaactcatcaagtctatgacaataaaggacatgggacaaaagcgctcagcaagcctgctctacttgtgattttttggttttgaactgcaaggtgggaaaagatgctccttacaatgttcgctctgcaaacatttattccctgatttaacccatcactaccatggccactgccactcactgattcaccaaaattgggtaaatcattgtcttgtttttattaatttttttttttgagacagagttttactcttgttgcccaggctggagtgcaatggtgtgatctcagctcactgtaacctccgcctcccgggtttaagcgattctcctgcctcagtctcccgagtagctggaattacaggcgcccgccaccacacccagctaattttttgtatttttcgtagagatggggtttcaccatgttggccaggctggtctcgaactcctgaccttagg

SEQ ID NO: 74 - DNA, 2100 bp, Homo sapiens
aaaggcttccttccctcaacaaaaaggatctcaccattctttatattccaggtttactttctgatttacccgtacagtatcatagcctggaggctcctgagatgcattcttttttggaaggggcctgattttgaagtctgactcagccactgattagtgagcaggctttcctaaatcttcctttcctctctagtaaagtaggtcagatcgtaatctcttcctcacaggaccattgtgcaaattaaaggagataacgtgaatggtttggtagcaactcaatgaatgtatctgaatcctcccgtcaatttactctgctgtccattatagtcagttatgtatctgttttatcctgtctggtcgagaatggaatctgaggaccaaaactcctctcttactcatctctgtgcattttcctctttgcccctgccccttcctccaacccagtacctagcatggtttcttcatttatgtgtaaattttatcaaagtggtgtcagtctgtgcaaatcatctgctgaagtctgttaagaacgttcagcaatatacacttcaacagtaacagggacagtgtggaaaacccttgttttcttcctactctccaattcttctgcctctttccactgctttggagaggaggttacaatcagagtcatttcaccataagaaaatcatcaattttctaatgaaaacccccctccttaaatattcagccgtggcacaatccctaaaggagagaacacattaatgcaatgcacttggccaatttgtgagccctgacttgttgagcacagactcttccctgaccctggaagacaatcggcaggacccctgaataatgaagtatgaatgctcaccattgtgctatctctagctacaccagcttctacctaatcttttttttccttttcattgctctcctttgcaacttcctaaggatttggccattttccactgtgtccaacctacgaagctgtaaatacctgactgagaaacacaaaatgtgtttataggacttctgatggcttctcatctaaaccataaatgtgaaatgcatttcaacatttctcagaaaacaccccatcccccagattcaattggcagcacaaatttacttctcagaagacagcaccaaaatctcaacattggcattttgaatcaagcacacacacgcagctgcattgtgttaacaaaagtgaaacattattaggtcgcatactctgagtgacaatatcctcgaatgatcatttctgtgagaaattaagctatgagaaattcaatttggtagcatttgggcattacagacagctagtctgtttatatcagcaaggctttgatgttaagactctgtaactgcagccacaggaaaagcagactgaatacagggtggataaggtcacagatataaaaatcagatagagttctgttctattatctacatagtgtgtactttgggaagttacttaatatttctaagcctcagtttcctcataaaaataaaaatggcaagcaatatgaaaactatctaatagaatatttgtgacactaaattgtaataatgtatataaatcacttagcctagtatgtggcatttattaacactcaagcaaaagtgtaattttttaaaaaaagactcttatatcccttacatgacagaaatattaagaccaaaatggttactgagccctcaaaggtattatctcattctggctgagctatctggacctggagaaaagtctagaaagactctatttcactccaagtttcttgaccctatctttattttttatcttctatccactaggacctgtgatcaggccaggattaaccagtgttctctaggattaacgtttttggcagctggggatgattgcctaagataattgtttttgtgtctgcctctcctgctagaatgcaaactctcaagggcaggacatatgtctttctttatcctacctgttagtgatcaacaggagaaggccactgcttaactgttagtgtcaggtcagctccaagctggtacttcttaggaactcttttcttttctttttctttttccttccttccttccatttctttctctttcttccttccttccttctgt

SEQ ID NO: 75 - DNA, 2100 bp, Homo sapiens
cacgtgaggacacagcataatagtagccacctgcaagcaaaggagagaggcccaggagaaacaagccctgccagcaccttacttaatcttagacttccagcctccagaattgtgagaaaataaatttctgttgtttaagctatccagcctgtgatattttgttatggcagccccaataaactagtatgtgtataatgaagccctagacaacaagggactctcatttctccgcatatttgtagaactcatcccaattatatagagctcctactctgagtgctagacacagtgttaaacactttccctgtgttatctcgtttaaccattaagctgaatcctccaaaaccctttggaaatcagacttatctaagaaactcactattgtagtgaagctgttttaaagaagaattgaaggtatttttctttatcttataatctgttacattgtgttacattttaagataatactaatctaaggactgataacaatttaatttgccagaatcattaaaccaaataacatctttaacagtggctgctagacaggggcagctgtatattttaatgccatatttgggggaaaaaaaacagggtagcaaacatatctataaataagaattaattgctacaaattacctgggaagggaaaaatgtcaagttcatataaagaatattattgacccatggatttacagctatataataatttggtacctggtttatttcttaaagacctagcacgtttcttgttttctcctgctatattacgtgtacatggcgtttcaataatcaagcaaaaaagatgtatgcactatcttagtctttgttgtctaattaaaactttttatgcatagcaattgcttaccattttgcattatcaccagagctcatttctcatggaaaaaaaattagcatcagtttaaaagaatatttctttaatcaacagttctgattgtcagtagtaccattttgtagatagtttttagctgactaacaaatcttttattttattggctgtctcattttgctctcttgcatatttcacatttatggtctattcagacattctcctgttttgttaagtggaaatctgtgtggtctttgatgtaagacataatttatttgacaaggaaatatgagtctgtgccctgaatccacatttaactgatggattgagaaattttaaaattgcaacaagatagactctcctccagattgccgtactacttgcattttgcttatctatttgggagtgaatttacatatgtgtgtctatatacgaatatatagagagtcatacaaccatgcagctgtacttgtgcaatttttctactttgttaatagaaaatgcagtctcattttgttagtcattaatggttcctatagaaaatttttaaagaatttttttctgaaattaaattcaagatacttattatgttttatcttcatatagatagctttataaagagagtgatgtcttcaagtctgtactgctcgcttctcagcctagtaaatggaagttttgttagcatttcaagatttatatatttcatatgttctcccaagtctatggcccagttctggtaattggaaacttactttcagctcattccctctgctcagactacttgtcaattaacctttgcaaaatgatagttttaaaaaatatgactttcatatttcaatcatgttcattttcaatcatctcaaaatgtagaaattgaataacacccggggttctacagtgctttttacatatcatttaaggtttaaaacatctctttgatgttcaaatatgactgccatttatattcaatggatgagattaagtggttaaaattacttgtactgggcatgcccctgctttgtttataggtatgaacaaaacactaaggatttttcataaatatgcaccatttccattgatgtttttgactgctgtctgtgacacactaggtaggccatattaagtaatggggaagaaatcataggtcctactgtgatattaaaaatttacattttgatgaattaaatagagttgttgaccattctacactgttgattatatgaagggaaaaagctaacaacttctaagaataa

SEQ ID NO: 76 - DNA, 2040 bp, Homo sapiens
aaaggtaaaagagagaagcacagagacactaaatgtataagtaattattcatttagttttgtaagtgtcctggtttgcatttcaggaaaaagaacaaaatctatcaatcgacatttttgtgcctattacaattgtccctcagtatacttggtgtatcggttccaggactacctgtttatacccaaatccaggcacattcaagtcctgcagctggccctgcagaaccttcatatgtgaaaagttggccctctgtatacacgtgttttagatcctacaaatattgtattttagatcagagcttgtttgaaaacagtttgtgtataagtggactcttgcagttccaacccatgctgttcaagtgctgactgtactctcaagctagttcacattttctaatttaatcctcaaaacgatgccttgaggaaaaataataatgacagaagatgcagagagagggcactgaagcttgtccaagtttacaaatagggtctccatgcaagtgttcacaattttcatcttttaatttcaaaaccagtgattttcccccctacttttacccacatataataataaattttagggtttaattcattttcttgtattatattttcatcctatagttctccattttaaccctaaaattacagaaatatttcctgtggctgaaagaaattagttagaaactcttaggtattttaagtaagaagaatgctacatgcaaaaagtaaagtctgaagaatgtatttcaaccttgaaaaaaaaaccctcagactcagtctgcagtaaagttcattcaacattaagaagcagaagctatttaaagagtttaatgcagaacacggtagaaaagatgcccattgccaagtccttcacggctcaccataaatgtgaacagcaaagtgctatccattttaaaaatcaatcaaggtgatgcagtgcttaaaatgcaattgattttggctaatttgcaagtagaaaggtttagaacttgaataatttctttcccactttagctggttcaacagcctactcaatgagttgcctaattattagtcttgttttttttttttttttcatttatttactttagacaattcctgagtgagacaacttttgatttatttcaactggctttgggtagatgaaagagaccacaaaccaaaccaaaccaaaacaaaacaaaacaaaaagccaccaggcaggccaagcacgatgaagaaaaacttcaaatgcaagaactataattttcaaactgtgagtttggaattcttaaaaagcaaaaaccattaagtgactccatttcagctacctattagtgatcaggttgaaaaacaatgagaaaaaatggaaagaaaatcactgcaaattccattggggtagattagaaagatgttaaattactctttttatctgccttctctgattaggaagcctaaagagcttaccacctttccaaggagtcagcatgtcactttaatgttttgctgctgttgtttggttgattgctgtatatatatatatatacatatatatatatatatatatatatatatatatatacatatatatatatatatatatatatatatatatatatatatatatatatatatatgtattcttgctctgtcacccaggctggagtgcagtggtgtgatcttggctcactgcaacctccacctactaggctcaagtgatcttcccacttcagcctctcaagtagtggggaccacaggcacacatcaccatgttgggtttgtttgtttgtttgttttttgtatttttagtagacacagggtcttgccatgttggccaggctggtctcaaactcctaaactcaagtaatctgcccacctcagcctcccaaagtgctggaattacaagagtgagtcactgtgcccggcctttagtgttcttttaaatagcattcataatcttttaaaatatgttttacaaatgaaacattgtgtttgagtgacatgatggggaaggagcctgcagtccacaacgtctctggcttcctttcttgcattttctcag

SEQ ID NO: 77 - DNA, 2040 bp, Homo sapiens
ctcagttttaaatgtttccatcaatcagtaattcagctccagagttgccatagaaggttatgggaaaaaaatccttctgcttttccaatatcaaagagagagatgttctggaaggttttatttttgccaccctgtttctaatgcatttcgccttaagaataatattactcatctccagcaatcaggctcaaggaggaaatttgataccattctgtgggtcatccccagatctctgcaggcttctggcagatgtatgatgtagtgggcaccactaactttgtctgctagggtattagtaggacgtggtctactgaataccggacagacatttggaaattaaactaatcaaaataggctaatttcacgtgctaagcaatgcaatttccctgaatttgtagttccatgatctactttttcttctacatttctcttactccctctcttctccgtttacaacacaaaattcaacaaactgctaactctagctattaatatcccatagtatttctctaagcagcttcatagtcacagtttcagttaaggctctagggctttcattcctgaactacgtgtactacagatcccagttaaaatctctatccttcccacaacagtggttgaccaccttcctcaacattggatttggggtgataatgacaacattctgaacaaagctcattttttcccctaacccccagctaaatagaagaaatgttaatgttactcctctttagatttttttaattaagaatatttttaaaggatttttttatgatttaatgggacacaatgaaaagatattttgacaaaggtaaacatctgaaactgaggcaaagaattttagatgttgcctgtgtaaccattattccatgatcaaaagcccaatgtttaacatatcctgatttcatcattaaacaagcataagaaaaaaaaaaaagaaaaccataaaagtgaaatagattacatcttgtaataatacagctatgaaattctgaccagaatgaaaatatgagtatgaagagagtaatcatttgcttattaattcaaggaacaatttgccatttttcaagtattatgaaaataagagactgttggactctttttaaacacgcaggtttttcaaatgtatgtacagtaatattatagctctggtgaaaattttgatgaaaacaaaattttctgtcttcttttacttaccttgcccttttcaaaaaatagtgcttaatgttaaccaatggacagtctaactacctgaagcttttcattcagctttattttctcagcaactatggttgaaactgacaagttaggtgaaaggttgtgtaagtatccaggcaggggcaaaaatatcgagttatccccaaatactaacaagcacataggtagaatatttctaccaagttaaagagaataaaggaaccacattgagcagagctaccttattcaaggacctcattatctcaaggcaccccaattgaataagtgtaccattattcccttcgttctgtgcaaacagcagacgtagagcacaaagagagacgatttcagtgaatcacactgtaattataaatgccacattaaaaaacggaacaaaagcaacagcaggacactgtaacgtcgatggttaaggagggcaaacagagaaacattctcaaaggccacaataaattacatgatccagttctttgttacaggcaaattattggacaataagagagacactgaacacaacttatcagtggtaaagtaacttgcacacattctcctaccactgaaatattcctgttacctacacaactgcatttgtatatacaagcaagaattttgatgcactaagtaattaatatagctctctagtattttttttacctctgtgcttattctatctgggcaaggtgtggtaatagcaccttaaaaaataagatcagatttaggagtgaagaacactgcattggaaaaggtaattgctaattttattgtattttaattatttgaccatttgtgcacaaaattaaataatacttgcttcatctctatatt

SEQ ID NO: 78 - DNA, 2160 bp, Homo sapiens
gtgtattgctgtgaaaaaagtcacttcaaatttagtagcttcaaccaacaatcattttatttgctaccaattttttgggtgagaagtttgtgctgggctcagttggacagttctactgatttccctgtatatacctcatgcagcaaaagtcatcctggtggttctatgtgatggttaatttttgtatcgacttggctggatgatagtccccagatatttggtcaaacgtcattctaaatatttctgtctatgtgttttgggaatgagataatattttactggattattcttcacaatgcgggtgggcctcatccagttagttgaaggccttaatagacaataattgacatcagacctaagtaagaaggaattctgccagcagactgcttttggactcaaattgcaactcttccctgagtctcaagcctgctggcctaccttgcatattttggacttaccaagcttccacgactgtatgaagcatttccttaaaataaatgtacatatctgtctctctacacacatacatatatacacactctcacacacatcctgttggttctctttctctggagaaccctaacacactccactcagacagattgtctaaggtggcttcacttaagttttggatatcgactcttgccgtcactctcttgaaacaatgttcttagactctcatgtaaagaaactccatctagctgcaccctggcactgtcccaaaaaagtgaggataatacctgcaaggtttcttaaggattatgccttgaagtcatattttacttctgtagagttctattagatcaaacaagttacctggccaagcctagggtcatagtgggagaattttgagggccatatggcaaacagcccaccatacaatacattctcaaatggcttctcaaattttacattcttgtgaatgattctctcttcttgtgattaattttacacacattacttcaatataaatttttatctcattgcattttcagtttttttgtaatttcacatttatggtactcttacaatgtgccatgtgctattctaagtattttatgtagattaatttaattcttacaacaaccttttgaggtaggtaatactattttgttcccattttcagatgaaaatgccaaggcatagaaagcttatgtaacttgcccagtaacactcagagacttaatgtcacaaccagtacttaaatgtatactatctgactacagggtatgcatgcttagtcataatgttattaaaatatcatttgtgatgactgaggcatcatggcagataggaggcaggactagattgcagttccagacagagcaggagacagaggcttgcacattgaattttagctccagatcgactgcaagagcaaaccggtaatcctgagaggacccacagatcctctgccggaagcagactgtttctgcaggaccaaggagacaccacagatactgtgggtgtcccaactgcagaaattggaaagggagacccttctcttccaaacacacaccccactggagaagctgtttctgactttacctggagctgagtcaagttagagagctgagccaagtgaaatacaggggtaggggaagtagcggaaagaccctgggagctcgctgggtcccccaagcagcccatacctgcctggcaccacagggatccactgggaggttggccagagaagtagggggtaaaataccacaggcagaaggaattctctagctaaactctgtaacaatttgaacggggcatgaagcctcctggccagtacttcaaggagggtgtgaatccagcatgcagacctcacaggcaggggggaaaactaaagcccttttctttggcagccgggaggtggaaagcctcaggcaagttttcaagcagggctcaccctccacctggaaacagactccaggttgttgagggggacacggtgggagtgagactggcccttcagctagcatgtgaactaggtgaggcctgtgactgctggctttcccctacttacctgacaacctacatgactcagcagaggcagccgtacttctcctagtgtgtccggaattggtgggttcttggtctcgctgtcttcaaggatgaagccgcggaccctcacggtgagtgttacagttcttaaagatggtgtgtccg

SEQ ID NO: 79 - DNA, 2100 bp, Homo sapiens
cctcccacaaggtccaactcaatcagaaccaaaagggagatcacagcatatccatgcaatccctggctggacagacggggcaccctagggcctggagttacgcagctgactggcagaggtcagtacccagttctacccagtgtggccacccgatccaactctgtgactcattttaaatcataagtagtttgaacaaagacttaaaattaacgggtttggtcatcattaaagtttatttttaaacgtcaagtaatttggcattttactaacaacaacttgaggatatccacagtgtatgaaacaccaactcttgtttcaataaaagtctaatgaaagtttctcaaattctgaaacctaattccaggattcttatttggaactgtttttttgtttttgtttttgtttttgttttatcagagtacattaactcagggggaaaatgagattatcttttgattcagagagaaacagaacattccactgatagtttaaaaataacacagtgaccacagataactgtagttcagcaattttcaaattcagattctgggtcagggcacaagattatgcgtctttaacaagcacaactgattataatgctgatggtctagaggaaacttgatgggaaacactgatctattcaacactttttaaaagtacactttagtactatttctacctagtcaataaaataaagaaggaaggagaaaggaggacgaataagagagaatccgaaagacacactacccagacagactactcagacagacgagacagtcaggcagacatggtgctacccgtcaagcatgcaggaaagcagctttcatggaataacattcctaaactcttgcctaataaattatgctgagagctgctgctaagaattttttaaaccaactcaaagcaaaaaaggagctattcacccaatcaacaggtgaaatcaagtcacagactagtatagggtttggcagatttcaggccctccagaaatatctgtttaattgagaagcaactccagctctagctagaaatctatttaaccataaaagtgaaatcataatgaatttggtcgtatcttatttttcccctttgtttgtttcctcggggcatctataatggctgaattggaaatggaaccacaagtattataacaacatttgttggaaagttcatcctgtattttagtagtacataagttgacagatatggctttatgaattgttctcagagacttaaaaaaaaaaaaaccctgaactttgtaaaaattacatccattatccaccaagtaacatttgcaagcaaaaccctctactagaaaaaatgggtgcgaaaataggaaaaggagaagaacaggaggaagaaaaggagaacaggacgtacaattaattgaggggaaaaaaatcatgagtaaagaagtcagaaataaatgtagctaaaaatacaaactgctactttatggtccagatattgtaatatatcatttttaacataaaagaaaaacaaatcctcaacagacttcctataaacgaaattatcagagttcccgagtacaccgggggtcgagggaagaatctccatgtgctccgagtatcgatagccagtccagcttcattcactcattcatttcttttctttcatttcaggagaacatttagcagtgttttgttttatttattttattcaaagggaaatcctcatgtgacactagcggtgaaaataacttgtatttgtaagttaatgtctgctgtacatctgagtacacaattgtctttcacagaagatggagcaaagtattacggaaagttcattggcttctgagtctgagagaaatgggttcaaatcctgaatacgttccttatctgtgtgatcttaagactcatcatttaatattctgagtcagtttcctcctctataaaacaagaatcagacggggcacagtggctcacgcctgtaatctcagcactttgtgaggccaagacggatgaatcatctgaggttaggagttcgagaccagccggactaacatgggaaaacccccgtctctactaaaaatacaaaattagccgggaatggtggtgtacgcctgcaatcccagctact

SEQ ID NO: 80 - DNA, 2040 bp, Homo sapiens
ttgattcatgggatgtttatgtggataattcctttgaaatccagcttgatttatgaacaatcttctctgctctattgagccattaaatccagagtattagtgcatttggaatacacagagatgataatgacatccaaagaagagtccagcaaaacttatttccatgaggactttttcagagggatgaagtaacattagctatacaggttagcattataagacttcccaagtgtagatgagaataatggcaactctgtggtcctaaggatgaatattgctctggaatatgcattttacactatatgaaagaaattaggatcgatataagctcacttatctttgccttattcctcctcatgttgtttttgtctagattgtctcagccacttgttttattttacttaaattttaatttcatcttattgtaaacctccattccttcagaaacaggtcaagaaacatgtcaatctacctaagtgaataactaatattaacaattaaataataaatagtactgaatgaatatacgaatacaagaataaataaaaataaaatgtattacttcatcgatggatttcctagtagatggggaaacggtgagaggatatgagcttcaataagaaaaatggtgcaaaataaggcagaagcaaatgcccaaaacaaatcaacacattcacaatttttccaaggacctgtcatgtatatatttttttctttttttaaccatttgtggcccctttttttaaccatttgtggcccctttcttatatctcatttctctcttttgtaaggcttctgtgttaattgacagcatgttcagatataaatccatcacaggaatgtgatgaaattagccattcagacccctgatattaagaaattcaaagaaatggatagagtatccaaccagtgaggaattaaagaagaaagaagagaaagagaaggagagagaaactagctgtaaagttgggatgggtcgggggtggtgaagaaaaccaattattcattgaaggtgccagaaggaaaattgatggcatgaatccatagcttctcaccataaaggtgaataatgacacagacacttagattggggaatgagaaaaaaaaggtgcatgcaaggttcttctatttatatctgattaagatatgaaaagaaaatgagagactggattactaaagaaaaattccagacagttaagcaattttaggaatgattcattttaagatatggccatcaattatttataagggttaataaatagatttataagcaagaggtacatggaatctagaaatacataaatgctcttcaaattatttacagctctgacaagtcataacacatgaactactaccaaaaacaccatttacttgactttaaaatttgcaccataaactataaatggaccagttatggagcatcagccatttgtaatgtgccatgcaatatttaacatcaactaaatgtgttttcactagctgctgaccacttggattaatttaataagcatgcctagtgcctaatgatttatttgtgggtaaatgatcataactatttaatggccttaatattacagatgtaattctgaaataaaatatcataacttggatttagtacatccagttaaataacaagcatcgacatttttaaaaaataataaaaacagtggccagaaaaagaaattaaagcacttgctagtcatatgtccccataggtttccagcttcatattggctttatttctttttttcctttcatttaggtcacccattaattttctttcttcatttgcacaccctcttccatttcctgtactatcttttgttctaatcctctagtaattccccagtgagctctcagcttccaaagggcactctatttctattaagcatggcagtcaacaagtggaaatagtccttggttgtcctgctttctgggtgaatagcagagtccctttgcatcacctcaaagactctgattctcatgatcctcagtctggtgctgaattgtgcttttgctcatccacacacatcccctacc

SEQ ID NO: 81 - DNA, 2100 bp, Homo sapiens
actcagcaatgggtagctatacttgagataataggcgggattttatgtgcagcaatgtagaggatgcaggggcccaggatgggctgccaggtcttccagagagtgctaaggtatccaccaaggatcatgaatgtgaacaagataatgaatcactgtctacttactcttttggaaaagcttccatatctctgccaattgaatcacactataaccagtcccaggcaattcaggatgacaagttccacttcgaacagttctgggagtcatcctgagggtccctgtgtatagacataaaaagttccatttgttcttacacagtgaaaatgacagaacaaatattatggggattatgcctggggaaaaaaaatctgtctctggatattcctgacactatggagagaaaatcagcaaaatttagaatcttggatctcttccactcacactaggatgttgtttctagaaatctccctgaagtatggtactgactcttggtggtaaaagtggagaggcttagaactgaaatctggtcagtagaagactgagggttaaaagtggacggtcaacccattgaatgaaggcctagcaggaaatagagagacaaaaatacaggcattaagggaataatagctgaatagtaataataatacattatgtcaacagcggtgacaaaggaaacactcaatgtatttatagagctaaataaacggcagatctaggtcctacgttttgactctgaacaaccttctcggttggattttgcttctgcctaaggattattttggaaagagctattattatccgtgatttatcacgctgcactggggggaactcatactttccacggagacaattactgaattctcactggaggcgcttaaaggagccaggacctgttctgagggttcaggtgggaaaggtgtgccagcaggggactgcagcctggcaccatgggacgtgtgtgctgttgaccacttctgtgcccagatgcctcaggcgctttctcattagatgcacccttcaatctcctggttattgaaacaggactggggagaggagttcacatttatggtgagcccatgcaagaagaccctccgacaggtgcctgtcacccctgaggagtcactggttgcagcccgttctgaagtgtcattgagatagaaaccaagtcaaagccgtggcctagaaagagagtctgggcagaattctgcaagcagattctttatttgagtaagtattccttgaagaagcccagttgtgcagctgtgtttgggtggaggtcatcagaggtttagaaaaagagagaagtcatggttaatattagaaaagaactctgagaatctggaggaaggaaaatgcattactagttctaagcaacaactgtggaaataaacaatgataaataccgtattaaatctaaagagttacgttaatagataataacaagtaggagagctaatagctagccattaatacaggccaatttattatttaaaacatttattaagatttaacaatagtcaaataatttttttgtgaaacagttattaaactgaatctctgcatacattaatcaactgatatttatcattcagaatgtatctcattatatccaaaagggtgtgtgtataggcttcaaaaacaaactggaagatttaaaatgaactgtagttcatttttgcaaagtgtagatgtgtaaagattattatgtttgccagctgggtagccagacagtgaagtggacttgtctaattaggaacaatcgctgataaatcaattctttccttttataggacaattacagtttgtgtgtatatgtgattgtgttttaaattctaattcgattttgtgcattgttctgtaaccaagttaattctttgaagcctttttaaatggtacaaattttccataaaatataaataggtttattgctgttttatcagtcacgcaaataatccaagatccatctattcacataattcaggcattaactgtgtataattactcacatgaagtcttcagtctggtttactatacggaaccccaaatatgactttaaattgctcgcctcctcttttcctctgttatttctctccctctctt

SEQ ID NO: 82 - DNA, 2103 bp, Homo sapiens
ttgcatcatctgacctctcttcgagtccaaaggactgagaactagaagaactactgctgtaagttccaaagtcccaaggcccctgaactaagatctccaatgtatgagagcaggaaaagatggatatcccaatttaaggagagagaaagagaaaatttgcccttcttgttctatttagatcctcaaggaattggaatgcccaaccacattggtgagtgtgggtcttctttacttattctactgattcaaatgttaacagattctggaaacgttctcacagatacacacagaaataatgttttgccagctctctgggcagctcttagcccagtcaagtttacatataagaataatcaacccagctttttataatcatcttagtatttaatcaaggaaatgatatcagctatctactaccacaaaaataattttaaaaattggcttataaaagatgacttagtggccctcagctgggacagcttgtcactatccaaccagcctttaaatccttccaagaagcaatcctggcttgttctcctgaaaactgggtagtttttcaagagattgagcagaagcatacaaggcctcctgacacctaggtgtgagatgggcacacaaccactcctgtcaaattctattggccaaagcaagtgacaaagccaatgcagatttaagaggtggtggaaaaaaaccctaaaaatagaagttgttgaaaagtcatatttcaaaagtcattggtataaagtagtgaaaaatttgacatttttgcaatcaagcttatcaaacaatattatcccaaaatataacaatacactcagtttgcacacttgtttaccttttgcaaaacaggtaagacagtaggacaaagcaggtgctttatgttgtttcaatcattcaggatttggacagtttggatattttctgtatcactataattgataaatactcagatgattctatagttaagttagaatggaaattttgggtatagtaacaaatactactttaatttaaacttacatgtaaacagtttccctaaagcagttagaagtgtgaccataaaagtgaaatggtttaaatacatgcatttacatctgtcctagagtgattaatgtaactttattataaaactactaattttgtgattacatacacccttccaaagatacattatacattcctatgtacactcaaatattatttttaaacttccattccaatcattaagtagaaatgcatttaagaatcatgatttttttagagtaagtctataggtggtacttttatattatagataacatttcctataccctttccacataaacacaagaacattatgctatagattgaaaattcctgtaaacactaagcagagcttttgtacataaacttgtaaaaactctacataaatgtattcagaaatacatgctattaaaatattttattgtatattactgtttggaagttttcagcttaaatatttttatttgatgatcaataagatctagtattaaatggtcttatttattaaccattaagttaaatacatggagaaatccactatgtcttttcctccagcctgtaagtaacacagggttgcatttctaatattaactaagttacatgtattttccattgagaagagtgctatcgaactcatccatgttaaatcactcttatgtggaaaaggctaacatataaataaaaaaactagaaaatttaaaaaaggataaagaaagaagaaaaatgaacagaatttaacagcagtgcaacagtagtctcttcctacctttcctgggcatcttccaattttatggtggtctgataagctttccaaaacactttgctcatttccagcactggacatttacactcaagactgcagactcgaggagtcacacactcagcatcttttagctgtatgttgtcaagttcagactactcaaagtggcatgtctttaaattagaatgtgtcaagtgggtctagtaactgcaccgaaatattttaatagtcatattaatcattaataagtcaggaaacatgtttttctaattttcagatcccaatacacatgactgatatggtttgcatctgtgtccctc

SEQ ID NO: 83 - DNA, 2099 bp, Homo sapiens
aaggtttggaaagcttaagtcaaaatgtgttgttcataaatacggtctgaataatttgaacattttctgttaatggtatttgttcaactataatgatattttccagccaagatataattggcaatgtcaaagtcacacacagatgggtaaaatggccaatgtctctggaaaatcttgataataactttttagtatctctggtgcaaggtcacttaaattcagaaaatagcacccaaggaaaaaatagccatattcaaaaaaataagctccatatatttagatgtagatataaatttggggtgatttatttcttattagacactaatattttttaaaacagagaatgacaaataaggaaattttgcagttaactatgtcctaatgaaaaagggtagtagttttacaagaaagatataattcatcaaaaaggcagggaagcattcagactaaacattgagtatgtttggaaataataaaaattattgtttcttttaccaacatcaacaatcttttcaaattaatttataaaactgtcatctctgttcactaattttgaattactcatattatttttaattttgaatacttataatattacttactaatttttaattaattttgatatacctatatcactgttttgaattgatctatgaatgatctagaaatgactttgcctgtttttttttttgactcatgggtatttacttttcattaggtaattttaatgtattgttaactagaaaaataagatgaagaaaaaaacattttaaatgcaaaatataaatttaaagaacttcaaaagaataaaatttcagttttatgtctttcaagtaaatttgctgttttcaaaattattttttgttacaaacctattttatttcaaaaaatatgctattgtttttaacctataatttttaaatatctgacagcattgtaggacttaaagctattaaatatataaagatataatagaacttattggaaatattcaaggaaaaactaacatattctttaaaaacattttaatttttaaattctatgttaattgactttttgatacatattttacttttccttcacttcttttgtcaattcttaaaaatgtctttcttcataatttttggcaattagtttttacactttaatagcaaacattgccataaaagtgaaattaagcattaattaattttatgtctgcaggcagagtgatttccttagggaatcaatttaatagagagaactatgtttgtacctggcaggatattcacagaaataaaatatttattggccatctactttgtttaagacctcttaacaaaccataacttattaaagcataaagtaacatacatagtaaatacttttaaaatctgtaaacaactaattcctttcttcttgtgaagtcttgtttagatcattaaagtaatagcagattttctcacaacaggtttgtgaatattgtctgtttaacatgaaaactataaaaaaattaaagacaattgatatatatttattcaactatgtcaactcaaagatgatctgcaattgttttctgaataacttattaataatgcttaggcccctttgttgaacatgcttttatttgtgtaaataagaattcatttaaaaatacattgtacaacttcaacacattgtgtgtccctgaaggtactctgagattttgcagttatagtataaatgagacaaaacggcagagaaaatattccccatgtgtaattctttctacatttatttcccacatcaatctcacaagtgtttttatttcacactgattgatatcattgagcacatacctcaatatctattatcacaaaaactatcattatcaacaaggactttaaaaaatatctaaacattattatctgggtagcaactctatactccattttatccattaattttgtctaattagtaaagaagtacttatggtaaaaacaaattaaaaatagtacagaaaacatactcctgtatgcaattattacaaatattttatttagttcctataaagtatttacatagctgagatcactatataatattatactcatgttactttatgtcctaactttatatca

SEQ ID NO: 84 - DNA, 2040 bp, Homo sapiens
cggctctcctggcctcgcgctgcacattctctcctggcggcggcgccacctgcagtagcgttcgcccgaacatggcgacacggagcagcaggagggagtcgcgactcccgttcctattcaccctggtcgcactgctgccgcccggagctctctgcgaagtctggacgcagaggctgcacggcggcagcgcgcccttgccccaggaccggggcttcctcgtggtgcagggcgacccgcgcgagctgcggctgtgggcgcgcggggatgccaggggggcgagccgcgcggacgagaagccgctccggaggaaacggagcgctgccctgcagcccgagcccatcaaggtgtacggacaggtgagcagttttgcaacccgcctccctccagttttttcctctccctgcacttcctcacccccgcatccatccgttgcagtcgcctcctaggtgcaggcaccactggggacttcccggcttgcatttgtttttttccttcacgagtacaaccgtcagcacttgaatcgcattgatctttccttcttcctgtcgatttagtaaacgtattccaggtaactcgccgggtgcagtgcgtattaccccagggtgtgtgcagagagatgtagtttccggcaggtataggaggggtgcagcttcattttacatctggataaaaaacgggctttctttagtgtatcatcagttggcagtggaggcgagcaccctgcagttgcggtacacttacacagaacagcacgaggtgggggtttccacacttagcattattagcacaataaaagtgggcaaacctgaaagcttgtcgactatctctgtacagtcagacaagaggtgtgtgtatgtgtgtgcgtgtgtaaaggctgaatttttaatttttaatttttggcgagcgtgtgagatgctctccattccttcttccccacccttcaagatgctgactctcccacccccgtcaagataactttattttggagaggaatacccctcatggcacttggagatttgaaaggactgcaggaaatttggtgggcattattattctataagtgatttatttctacccaggcaataggtttattagatcataagtaacgtgaatttcacttttatggtcagacttactgcgaggaattgcagatggagtttgtaggttaggatcagcactggcaaaattaatttgaccgtgttattgcctcatgagactcccagtcctgcagttaagattgacatcagcaaaagtataaggtcggtgggggagaaaaagtaggaccagaggagggggtaaatacacttgttttctagagtcaaattgttccttttgaagtagaaattattaataaaagattaccctgagttctgccttttctcactaatttcactttagccatttcttcaggaaatacagagttaaatgttcaacccttggatccaggacgaaccttgtaaacatatcaccctattgtgtcattttgttggtgaagaaactgaagcgtggaatggtgaagtgactagtccaaggtcataccgggaaggtggcctgctctctagtttttgtctgcattgtctcagtgacctttgcttgactgcagtcaccctgtctttatgcaatgctgctgaaatacctcctttctaaaataaaatagatctggtataaagggggaaaggatggtggtgactgggtgggagcgttggatttccctccactattggtccctgggcaagaatgtgtgccccagggcatgtaactaatggtggccacaggctgcaggaacctgcatgctcagttcctcttgggcccagatccttgtccccctgtccccaccccatatgacaaatatgtgtatgaacaaaaagaagtcatcaaggtccttgctcttaacagcgacaccagcatggggctgatggagggtgggagaaggaggaggagtggcccacttcttcattgggcctccgcagtcagcccagctctgctgtgctcttgaatcagcattctgggaactgggagttgggggctggtgggagacaa

SEQ ID NO: 85 - DNA, 2099 bp, Homo sapiens
acaaaaggcaaattggtgtctctgtcctggagtccttactcctcatcttgtgcttagacatgaaattacacatctccagccttgggattccaggacttacaccagtagcatgcctatgttctaaggcctttggcctgggactgagaattacaccatcagcttttctggttctaaggcttctggacctgaactgagccatgctaccagtatttcaggatgttcagcttgcagatagcctgtcgcggaacttctcagcctctagaatcacatgagtcaattcccctaataaatctccttttatctatctgaacatctctcttcatctctccatccatccactcatgtgtccatccatccatccatctattgctatctatctatccatccatgcatccatccattcaaccatccatccacccatccatccatccctgtgccatctatatctatctatctatatatctatctatccatgcatccatccatccatctatccatctatccatccatcactatctatccatgcatccacccatccacccatccatccatccatccatccatccatcactatctatccatccatgcatgcatgcatccatccatccatccatccatccatccatccatccatttatcgctatctatctatccatccatgtatccatccatccatctgttcatctatcactgtctatatatctatgtatctatctatccatccatccatgcatccatccatgcatccatgcatccatctatcactatccatccatccatccatccatccattcatccatctatctgtcttctacctacctacctatctaactctctggagaactctgactaataaactagctttaaaacatgttattctctctctgcaatgtctattgctttatcttcaggaacattccacacatcctgtaagacttcagttaaattatctctctgtttcttctccaatcatcctctgccttccctagtctcctaacgtactttgtacatctgtcacaaacccctcatcatatttactgtaatttttttcctacagatttggataggaattgagccatttttttaatttcacttttatggttgttacaaataaaagagcaagcaggcccctcactgtaattcacctgtatttgcatttaacttattaaccaaggcatactatttcaaataatctaatatagtatttcctatttaataaccaaacatacagaacagttccaagcacatgtaaccatgtgatacattttcctctttgaataataaatatatttcttataattaatatgtgataaaattgcaatatttttaatctcctacatccttctcttttaatcaggtttccttatcaactggttcctatctcacggggttgttgcagagatgaggaaaaaaagtattctattggttcatgcatctcaaaataggcagattcttttctctgcttcttccttcattggctcaggtgtggagtgcttctcccaattatatgtgccagccttggtatgttctcattgctgtaccacactgcctgagacatccaagaccacatcttcctttgggggcacattggacctttgtcattggcactggcagggaagcttttatttcaccaggtctaaggcaattcttccaaaaaaatcccaaatagtgaaagaattgatttattcttctaatatttaagcaaatgtaaaaaaaaagttacattagttatgtttttttcagattttggatcagtgagacttcattaaaacactttgaggttataaagcaagtaatttttgtttccagaaaagttagtttcctttggctgaagggacatctctatgcaggccagatcaagacaaaaataacttttaagaagggaaatgagggaatggagtttggaaaacataaatcccacagcaaagtacgtcaccaacaataagagtcatctctttcacagaggcctttcctagaaaagccctgacagactaggagtccaatcttcggctcccatagcacccatgcctgcttccactctggagcttactactttgcgttgaaattaatttttacatgtctatggcttctattacaaa

(19) United States
(12) Patent Application Publication
(10) Pub. No.: US 2009/0023674 A1
(43) Pub. Date: Jan. 22, 2009

(54) snRNA gene-like transcriptional units and uses thereof

(76) Inventor: Aldo Pagano, Genova (IT)

Correspondence Address: JUNEAU PARTNERS, P.O. BOX 2516, ALEXANDRIA, VA, 22301, US

(21) Appl. No.: 12/066,829

(22) PCT Filed: Sep. 19, 2006
(86) PCT No.: PCT/IT2006/000664

§ 371 Date: Sep. 8, 2008

(30) Foreign Application Priority Data
Sep. 19, 2005 (IT) RM2005A000475

(51) Int. Cl. A61K 31/7105; C07H 21/02; C40B 40/08; A61P 25/28; C12Q 1/68

(52) U.S. Cl. 514/44; 536/23.1; 506/17; 435/6

(57) ABSTRACT

By a computer search for upstream promoter elements (DSE, PSE) typical of small nuclear RNA (snRNA) genes, we have identified a number of previously unrecognized, putative transcription units whose predicted products are novel noncoding RNAs with homology to protein-coding genes. By elucidating the function of one of them, we provide evidence for the existence of a sense/antisense-based gene regulation network where part of the Pol III transcriptome could control its Pol II counterpart.

The instant invention concerns sequences transcribed by the RNA polymerase III type III and their use for medicine, agronomy and biotechnology.

The author has identified an unknown transcription of encoding and unpolyadenylated genome elements that are synthesized by means of RNA Pol III type III promoters or of very similar elements. Other than their identification and molecular characterization, said new transcription units were functionally analyzed and their regulative features were identified. Each transcription unit is functionally related to a specific RNA Pol II transcripts giving rise to specific sense/antisense sequence molecules.

### BACKGROUND ART

Recent advances in mammalian genome studies are bringing to light the occurrence of a widespread transcription of non-coding (nc) regions devoted to the regulation of the protein coding genome expression [1-4]. The mechanisms of action of these transcripts are various and of different nature, although all of them are devoted to the regulation of fundamental genetic pathways involved in the determination of the cell phenotype. The concomitant evolution of non-coding regulatory transcripts and proteins that target different RNA:RNA or RNA:DNA complexes emphasizes the importance to study the regulatory processes mediated by nucleic acids interactions. It's now clear that either in procaryotes as well as in eukaryotes different ncRNAs can act in cis and be contemporaneously regulated in trans by other non-coding transcripts. The simultaneous occurrence of cis and trans regulatory elements bring to light the complexity of this network where the coexistence of different non-coding RNAs plays a key role in the control of other targets gene expression [5]. In this context a prominent role is played by the enlarging family of microRNAs (miRNAs) that act at post transcriptional level by inhibiting the translation of protein coding genes [6]. The known miRNAs, as protein-coding mRNAs, are synthesized as polyadenylated precursor molecules by the RNA Polymerase II transcription machinery [7]. Considering that the vast majority of the tools used in molecular biology are based on transcript collections obtained by oligo-dT RT-PCR (thus encompassing only polyadenylated RNA Polymerase II products) a wide contribution of non-polyadenylated transcripts to the human transcriptome has been shown [S]. However, the role of such transcripts in Pol II transcriptome expression regulation remains largely unexplored.

Among the non-coding elements one of the most investigated has been the Alu class of repetitive sequences that represents about one tenth of the whole human genome. Although it is not yet possible to discern a peculiar Alu's role these short transcripts has been shown to be involved in several biological processes such as RNA editing (where Alus are preferential sites for A to I RNA editing thus having profound implications either in gene expression regulation as well as in the mammalian genome evolution) [9], alternative splicing (internal exons that contain an Alu sequence are almost always alternatively spliced) [10], chromosomal recombination (the recombination between Alu elements is at the base of many genomic deletions associated with many human genetic disorders) [11], gene expression regulation (functioning as naturally occurring antisense RNAs) [12], cell stress response (such as heat shock response and/or translation inhibition) [13] and as putative miRNAs targets [14]. However, although the physiological role of Alus and all the other 7SL-derived transcripts needs to be further studied in detail, the fact that their transcription is RNA Polymerase (Pol) III-dependent bring to light a previously unexpected role in gene expression regulation of this enzyme that would need to be investigated in detail.

In this work we focus on a specific class of non-coding RNAs starting from a theoretical hypothesis on their putative function. In fact, starting from the observation that RNA Polymerase (Pol) III is specialized in transcription of non coding ncRNA genes, we postulated the presence in the genome of a large number of Pol III (or Pol III-like) transcription units each specifically regulating one (or more) specific Pol II genes, thus constituting functional “co-gene”/gene pairs.

### DESCRIPTION OF THE INVENTION

Therefore it is an object of the invention a nucleic acid molecule comprising a nucleotide sequence that is characterized by:

being transcribed by an RNA polymerase III,

it does not undergone any polyadenylated tail addition (as for Pol II transcribed genes) and

it is able to modulate the expression of one or more specific RNA polymerase II-transcribed target genes.

Preferably said nucleotide sequence comprises a sequence of at least 50 nucleotides that is at least 70% identical to a fragment of one of the strands of the specific RNA polymerase II-transcribed target genes.

More preferably said sequence of at least 50 nucleotides is in a sense or an antisense configuration with respect to the fragment of one of the strands of the specific RNA polymerase II-transcribed target genes.

In a particular aspect the nucleic acid of the invention is comprised in one of the sequences from SEQ ID No. 51 to SEQ ID No. 84, preferably the sequence of at least 50 nucleotides that is at least 70% identical to a fragment of one of the strands of the specific RNA polymerase II-transcribed target gene is comprised in one the underlined fragments of the sequences from SEQ ID No. 51 to SEQ ID No. 84.

It is another object of the invention an expression vector comprising the nucleic acid according to the invention.

It is another object of the invention an array for the detection of specific nucleic acid sequences containing a repertoire of nucleic acids according to the invention.

It is another object of the invention the use of the nucleic acid according to the invention to modulate the expression of RNA polymerase II transcribed genes.

It is another object of the invention the use of the nucleic acid according to the invention to identify a target sequence for treatment and/or prevention of a molecular pathology, preferably an age related pathology, including Alzheimer disease; alternatively the pathology is caused by an alteration of cell proliferation, preferably the pathology is a tumor associated pathology.

It is another object of the invention a nucleic acid comprising at least one sequence being able to modulate the RNA polymerase III mediated expression of the nucleic acid as above described, preferably the sequence being able to modulate the RNA polymerase III mediated expression of the nucleic acid as above described is a promoter sequence.

In a particular aspect the sequence being able to modulate the RNA polymerase III mediated expression of the nucleic acid as above described is comprised in one of the sequences from SEQ ID No. 51 to SEQ ID No. 84. Preferably the sequence being able to modulate the RNA polymerase III mediated expression of the nucleic acid according to claims 1 to 5 is comprised in the bold regions of sequences from SEQ ID No. 51 to SEQ ID No. 84.

It is another object of the invention the use of the nucleic acid comprising the sequence being able to modulate the RNA polymerase III mediated expression of the nucleic acid as above described to modulate the expression of one or more specific RNA polymerase II-transcribed target genes.

It is another object of the invention the use of the nucleic acid comprising the sequence able to modulate the RNA polymerase III mediated expression of the nucleic acid as above described to identify a target sequence for treatment and/or prevention of a molecular pathology, preferably the pathology is an age related pathology, including Alzheimer disease. Alternatively the pathology is caused by an alteration of cell proliferation, preferably the pathology is a tumor associated pathology.

It is another object of the invention a vector comprising the nucleic acid comprising the sequence able to modulate the RNA polymerase III mediated expression of the nucleic acid as above described to get expression or silencing of a RNA polymerase II transcribed specific nucleotide sequence.

The invention shall be described in the following non limitative examples, by referring to figures.

### FIGURE LEGEND

FIG. 1) A: Human CENP-F gene structure as resulting from GI:89161185 (region 212843155-212904537). B: The position of the 21A antisense homologous regions are reported together with their percentage of identity. C: Sequence alignment of 21A/CENP-F homologous regions.

FIG. 2) A: Northern Blot analysis of Human Skin Fibroblasts and HeLa cells. Results show two bands: the first (detected at about 300 nt) being the 21A endogenous product and the second (of a very high molecular mass) representing CenPF mRNA. B: 21A-specific RT-PCR amplification. As expected for non-polyadenylated transcripts an efficient amplification product was obtained only in the random hexamers-primed reactions. C: Promoter activity transfection assay. A specific luciferase silencing hairpin is transcribed by six novel PSE/DSE-dependent promoter elements (11A, 14A, 21A, 29A, 38A, 51A). pGL3+pRL: negative control; pSHAG-U6: canonical Pol III promoter; No Promoter: hairpin without PSE/DSE-dependent promoter thus resulting transcriptionally inactive. A schematic view of the silencing constructs including the hairpin nucleotide sequence is enclosed. D, E: Promoter activity transfection assay in presence/absence of 20 μM ML-60218 cell-permeable Pol III inhibitor or 10 μg/ml α-amanitin Pol II specific inhibitor. Results are reported as luciferase emission of treated versus untreated samples.

FIG. 3) A-D: Constructs structures. p21A: whole transcription unit; p21A-1: promoter region. p21A-2: transcription region; pMock; empty vector, p: PSE Element. d: DSE Element. t: TATA box. E-H: CENP-F protein expression level after 0, 24, 48 and 72 hours of constructs transfection. s: anti-CENP-F Antibody. 1: anti-Tubulin Antibody (Indicating that equal amounts of proteins were loaded). Striped columns: Quantitative determination of CENP-F expression modulation as determined by Western blot analysis. Full columns: Quantitative determination of CENP-F mRNA expression modulation as determined Real Time RT-PCR analysis. I-N: 21A RNA level in transfected samples indicating that the exogenous 21A expression inversely correlates with CENP-F protein expression. O: Dissociation curve of 21A amplification products. A: 21A-transfected HeLa cells. B: Untransfected HeLa cells showing the very low basal 21A transcription level.

FIG. 4) A: Proliferation inhibition of HeLa cells after 48 hours of 21A constructs transfection. Results emphasize the specificity of the Alu Jb-containing regions as proliferation inhibitors. B: Proliferation increase of HeLa cells after 48 hours of pAnti-21A and si21A transfection. siEx-FABP: unrelated chicken-specific siRNA (negative control). C, D: Anti-21A construct structure: the transcript region is inverted and the construct maintains 21A promoter as well as its termination site. si21A:siRNA 21A-specific. CENP-F protein expression level after 0, 24 and 48 hours of constructs transfection. s: anti-CENP-F Antibody. 1: anti-Tubulin Antibody (Indicating that equal amounts of proteins were loaded). Striped columns: Quantitative determination of CENP-F expression increase as determined by Western blot analysis. Full columns: Quantitative determination of CENP-F mRNA expression modulation as determined Real Time RT-PCR analysis.

FIG. 5) Mouse NIH-3T3 cells proliferation rate after transfection of 21A constructs. No proliferation decrease was observed.

FIG. 6) Real-Time RT PCR analysis of 21A endogenous RNA in different cell types. Striped columns: 21A RNA; Full columns: 5s rRNA. The dissociation curve of 21A amplification product in PBL is reported.

FIG. 7) Graphic representation of the total number of DSE consensus sequences in all the putative promoter sequences (Y axis) versus the distancies from their neighbouring PSE elements (as grouped in 50 bp long sequence classes) (X axis). As expected a high frequency of DSE consensus is associated to the distance of about 200 base pairs from the PSE. As evidenced by the trend line (polynomial) the DSE frequencies significantly decrease at about 800 base pairs upstream the PSE; this roughly suggests a PSE/DSE functional relationship in these putative promoters.

### MATERIALS AND METHODS

### Databases and Searches

All the sequence searches and alignments were carried out taking advantage Basic Local Alignment Search Tool of the National Center for Biotechnology Informations (www.ncbi.nlm.nih.gov/BLAST/); The sequences used as query were the following: H1 PSE-nCACCATAAAnGTGAAAn (SEQ ID No. 1) or nTTTCACnTTTATGGTGn (SEQ ID No. 2), U6 PSE (Acc N°: M144S6) CTTACCGTAACTTGAAAGT (SEQ ID No. 3), 7SL PSE (as reported in PMID: 2011518) TTGACC-TAAGTG (SEQ ID No. 4), DSE (Oct1 consensus sequence)—ATTTGCAT (SEQ ID No. 5) or ATGCAAAT (SEQ ID No. 6) with or without a single base of mismatch.

### Cell Culture, Transfection and Luciferase Assay

For transient transfections Hela cells (grown in DMEM supplemented with 10% FCS), were grown in multiwell Petri dishes 16 hours before transfection. The expression [2]A, 21A(1), 21A(2), 21A(3)] constructs containing the regions of interest cloned in the pTopo vectors (Invitrogen) were introduced into the cells using the Fugene 6 transfection reagent (Roche) according to the manufacturer's instructions. A plasmid Expressing Luciferase was used as control of transfection efficiency (to which all the results were normalized). 24, 48 and 72 hours after transfection cells were harvested and firely luciferase activity was measured by Dual-Luciferase reporter assay system (Promega). manufacturer's protocol. In order to specifically inhibit RNA Polymerase III and/or RNA Polimerase II, a cell-permeable chlorobenzenesulfonamide (ML-60218) (Calbiochem, California USA) and/or α-amanitin (Roche Diagnostics GmbH, Germany) were used at the concentration of 20 μM and 10 μg/ml respectively in the medium for 25 h (ML-60218) and 12 h (α-amanitin) before the luciferase activity detection.

### RNAi-Silencing Assay.

In order to test the promoter activity of the novel transcription units we prepared six plasmid constructs expressing a firefly luciferase silencing hairpin (obtained by Gregory Hannon's Laboratory-Cold Spring Harbor Laboratories) which transcription was driven by the 11A, 14A, 21A, 29A, 38A, 51A promoters respectively. The hairpin sequence [targeting a firefly luciferase mRNA from a co-transfected expression plasmid (Promega)] is:

5′GGAUUCCAUUCAGCGGAGCCACCUGAUGAAGCUUGAUCGGGUCUCGCU
GAGUUGGAAUCCAUU-3′.

Oligos used to subclone the novel Pol III Type III promoters within Not I/HinD III restriction sites (in capital) were the following:

11AFprom Not I:

(SEQ ID No. 7)

5′-atgcGCGGCCGCatttgcatgtcgctatgtg-3′
11ARprom HinDIII:

(SEQ ID No. 8)

5′-gatcAAGCTTcatcaggtggctcccgctgaattggaatccacgcact
cagctcgtg-3′
14AFprom Not I:

(SEQ ID No. 9)

5′-atgcGCGGCCGCaactgatgtatgattatatctt-3′
14ARprom HinDIII:

(SEQ ID No. 10)

5′-gatcAAGCTTcatcaggtggctcccgctgaattggaatccattatta
tctcctttgttctgt-3′
21AFprom Not I:

(SEQ ID No. 11)

5′-atgcGCGGCCGCacagctgtagcagatgct-3′
21ARprom HinDIII:

(SEQ ID No. 12)

5′-gatcAAGCTTcatcaggtggctcccgctgaattggaatccaccacac
ttggtcaactat-3′
29AFprom Not I:

(SEQ ID No. 13)

5′-atgcGCGGCCGCttctcacctaaaggagtc-3′
29ARprom HinDIII:

(SEQ ID No. 14)

5′-gatcAAGCTTcatcaggtggctcccgctgaattggaatccttctaat
cctcctaagatca-3′
38AFprom Not I:

(SEQ ID No. 15)

5′-atgcGCGGCCGCttcactaagatccagtgc-3′
38ARprom HinDIII:

(SEQ ID No. 16)

5′-gatcAAGCTTcatcaggtggctcccgctgaattggaatccgattcat
gaacacagaatatt3′
51AFprom Not I:

(SEQ ID No. 17)

5′-atgcGCGGCCGCgttgaacatttaactctgtat-3′
51ARprom HinDIII:

(SEQ ID No. 18)

5′-gatcAAGCTTcatcaggtggctcccgctgaattggaatccctcatgg
cacttggagat-3′

In this analysis the above constructs were co-transfected with a pGL3 plasmid (Promega) expressing Firefly (ff1) Luciferase as target to be silenced and with a pRL plasmid (Promega) expressing a Renilla Luciferase to which all the determinations were normalized. 24, 48 and 72 hours after transfection cells were harvested and firely/Renilla luciferase activities were measured by Dual-Luciferase reporter assay system (Promega) according to the manufacturer's protocol.

### Plasmid Constructs Generation and Sequencing

The plasmid constructs p21A, p21A(1), p21A(2), p21A(3) were generated amplifying from a genomic DNA preparation the regions of interest; the PCR products were then subcloned in a pTOPO Vector (Invitrogen) following manufacturer's instructions. The oligos used to generate p21A PCR fragments were the following:

(SEQ ID No. 19)

| | |
|---|---|
| 21A Forward: | 5′-GGAAATCTTACCTTCCTGCC-3′ |

(SEQ ID No. 20)

| | |
|---|---|
| 21A Reverse: | 5′-TGGCTAGGTCATGTGACCAT-3′ |

(SEQ ID No. 21)

| | |
|---|---|
| 21A(1) Forward: | 5′-GGAAATCTTACCTTCCTGCC-3′ |

(SEQ ID No. 22)

| | |
|---|---|
| 21A(1) Reverse: | 5′-TTCATTCATTCATTCATTGATTCAC-3′ |

(SEQ ID No. 23)

| | |
|---|---|
| 21A(2) Forward: | 5′-CAGCTGCAGCAGATGCTAGCAGGGC-3′ |

(SEQ ID No. 24)

| | |
|---|---|
| 21A(2) Reverse: | 5′-TGGCTAGGTCATGTGACCATTC-3′ |

(SEQ ID No. 25)

| | |
|---|---|
| 21A(3) Forward: | 5′-CAATCCTCAGAAATTTTCAACTGCC-3′ |

(SEQ ID No. 26)

| | |
|---|---|
| 21A(3) Reverse: | 5′-TGGCTAGGTCATGTGACCATTC-3′ |

The plasmid constructs pAnti-21A was generated amplifying the transcribed region from p21A plasmid using the following oligos:

Anti-21A Terminator-containing Forward:

(SEQ ID No. 27)

5′-CTGAAAAAGTAGTCCCAGCACTTTG-3′
Anti-21A Bam HI-containing Reverse:

(SEQ ID No. 19)

5′-ATGCGGATCCGAGACAGGGTCTTGCTC-3′

thus generating the transcribed region in anti-sense configuration. The pAnti-21A promoter was obtained by amplifying p21A promoter with the following oligos:

21A Forward:

(SEQ ID No. 19)

5′-GGAAATCTTACCTTCCTGCC-3′
p21A Bam HI-containing Reverse:

(SEQ ID No. 28)

5′-ATGCGGATCCGAGCCACCACACTTGGTC-3′.

The PCR products were digested with the restriction enzyme Bam HI, purified by gel electrophoresis and ligated by T4 ligase (Invitrogen). The insert obtained was then subcloned in pTOPO vector (Invitrogen) following manufacturer's instructions. Prior to transfection all the plasmids were sequenced by DNA Sequencing Kit (Applied Biosystems) following manufacturer's instructions.

### RT-PCR Reactions

In order to isolate and sequence a partial 21A cDNA ve performed different RT-PCR reactions. Starting from about 5 μg of total RNA, cDNA was synthesized by using an Oligo(dT)₁₂₋₁₈ primer or a random hexamers mix and a Superscript first-strand synthesis system for RT-PCR (Invitrogen). cDNAs were diluted 10-50 times, then subjected to PCR reactions. The oligo used to isolate 21A RT-PCR product were: oligo forward 21AF 5′gctcacgtagtcccagcacttt-3′ (SEQ ID No. 29) and oligo reverse 21AR 5′-actatgttgcccaagctggtct-3′ (SEQ ID No. 30).
PCR products were separated on 1.5-2% agarose gel. The DNA bands were cut, purified by the DNA Gel Extraction Kit (Millipore) and sequenced.

### 2.8. Real-Time Quantitative RT-PCR

The RNA for 21A was measured by real-time quantitative RT-PCR using PE ABI PRISM@7700 Sequence Detection System (Perkin Elmer) and Sybr Green method. The sequences of 21A forward and reverse primers as designed by the Primer Express 1.5 software were 5′-GCTGAGGCAGGAGGATCACT-3′ (SEQ ID No. 31) and 5′-GCACTACCACACCCAGCTAATTTT-3′ (SEQ ID No. 32). The sequences of CENP-F forward and reverse primers were 5′-CTGCAGAAAGAACTCTCTCAACTTC-3′ (SEQ ID No. 33).
and 5′-TCAACAATTAAGTAGCTGGAACCA-3′ (SEQ ID No. 34). For endogenous control the expression of Glyceraldehyde 3 phosphate dehydrogenase (GAPDH) gene was examined. The sequences for human GAPDH primers were 5′-GAAGGTGAAGGTCGGAGTC-3′ (SEQ ID No. 35) and 5′-GAAGATGGTGATGGGATTTC-3′ (SEQ ID No. 36). The sequences for human 5s rRNA primers were 5′-TACGGCCATACCACCCTGAA-3′ (SEQ ID No. 37) and 5′-GCGGTCTCCCATCCAAGTAC-3′ (SEQ ID No. 38). Relative transcript levels were determined from the relative standard curve constructed from stock cDNA dilutions, and divided by the target quantity of the calibrator following manufacturer's instructions.
Anti-21A siRNA Synthesis

The Anti-21A siRNA was synthesized against a region of the 21A transcript of no homology with CENP-F so that the silencing effect was specific for the Pol III regulatory RNA and did not interfere with CENP-F mRNA stability. The siRNA synthesis was carried out taking advantage of the siRNA Construction Kit (Ambion, USA) according to the manufacturer's protocol. The Sense/2Antisense oligos used were: 5′-aaGTGTGGTGGCTCACcctgtctc-3′ (SEQ ID No. 39) and 5′-aaGTGAGCCACCACACcctgtctc-3′ (SEQ ID No. 40).

### Proliferation Assay

We tested proliferation of HeLa cells transfected with 21A, 21A-1, 21A-2, 21A-3, Anti-21A constructs plating 5×10⁵ cells per well in round-bottomed 96-well plate, incubated for 24/48/72 hours after transfection and pulsed with 3H thymidine (1.0 μCi/10 μl/well) (Amersham Biosciences) for the last 18 hours. We harvested the cells and evaluated cell proliferation by counting the thymidine uptake. We calculated the averaged proliferation rate, measured as counts per minute (cpm), and standard deviation (SD) for the triplicate wells of each sample.

### RNA Isolation and Northern Blot Analysis

Based on a single step acid-phenol guanidium method, total RNA was extracted using TRIzol reagent (Invitrogen) according to the manufacture's protocol. Total RNAs, from HeLa cells, were electrophoresed through 1.5% agarose gels in the presence of formaldehyde and blotted onto Hybond N membranes (Amersham). The blot was hybridized with an 85 bp long probe contained the region from nucleotide 1194 to nucleotide 1278 of the 21A reported sequence (see Table 1) spanning a region internal to the transcript and complementary (96%) to part of the CenPF mRNA. The probe was obtained by PCR (using the 21A plasmid construct as template) using the following oligos: 21AF 5′-GCTCACGTAGTCCCAGCACTTT-3′ (SEQ ID No. 41); 21AR 5′-AGACCAGCTTGGGCAACATAGT-3′ (SEQ ID No. 42). Blot prehybridizations was performed at 65° C. for 2 h in 333 mM NaH2PO4 pH 7.2, 6.66% Sodium Dodecyl Sulphate and 250 mg/ml denatured salmon sperm DNA. Blot hybridization was performed at 65° C. for IS hours in the same solution containing 10⁶ cpm/ml of denatured and labeled probes. After hybridization the blots were washed twice at 65° C. for 30 min in 0.2% sodium dodecyl sulphate, 2×SSPE and once at 65° C. for 30 min in 0.2% sodium dodecyl sulphate, 0.2×SSPE. Membranes were exposed to autoradiographic films for 24/48 hours and then developed.

### 2.4 Real-Time Quantitative RT-PCR

Total RNA preparations from different CENP-F (Centromeric Protein F) (Acc. n°NM016343) samples was subjected to reverse transcription by SuperScript II First Strand Synthesis Kit (Invitrogen) following manufacturer's instructions. The cDNA obtained was measured by real-time quantitative RT-PCR using PE ABI PRISM@ 7700 Sequence Detection System (Perkin Elmer). The sequences of forward and reverse primers as designed by the Primer Express 1.5 software were 5′-CTGCAGAAAGAACTCTCTCAACTTC-3′ (SEQ ID No. 43) and 5′-AGTTGTTAATTCATCGACCTTGGT-3′(SEQ ID No. 44). The TaqMan™ fluorogenic probe used was 5′-FAM-AGTACCTGTTTTCTGCTTCTCCTGTGCAGC-TAMRA-3′ (SEQ ID No. 45).

The probe was placed at the junction between two exons. During PCR amplification, 5′ nucleolytic activity of Taq polymerase cleaves the probe separating the 5′ reporter fluorescent dye from the 3′ quencher dye. Threshold cycle, CT, which correlates inversely with the target mRNA levels, was measured as the cycle number at which the reporter fluorescent emission increases above a threshold level. For endogenous control the expression of Glyceraldehyde 3 phosphate dehydrogenase (G3PDH) gene was examined by quantitative RT-PCR as described above. The sequences for human GAPDH primers and probe were 5′-GAAGGTGAAGGTCGGAGTC-3′ (SEQ ID No. 46), 5′-GAAGATGGTGATGGGATTTC-3′ (SEQ ID No. 47) and 5′-TET-CAAGCTTCCCGTTCTCAGCC-TAMRA-3′ (SEQ ID No. 4S).
Relative transcript levels were determined from the relative standard curve constructed from stock cDNA dilutions, and divided by the target quantity of the calibrator following manufacturer's instructions.

### Western Blot Analysis

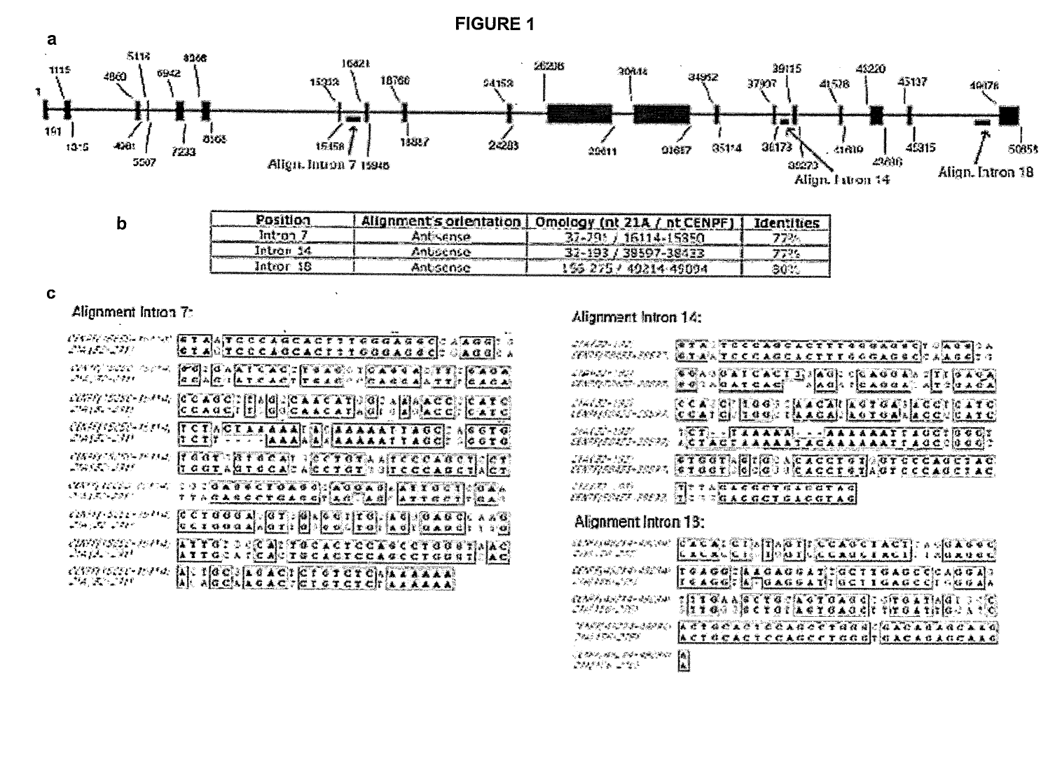

Equal amounts of proteins (10 μg/sample) from each sample were loaded on standard 4-12% NU-PAGE gradient gels (Invitrogen S.r.l., Milano, Italy). Blotting onto Protran nitrocellulose membranes (Schleicher & Schuell, Dassel, Germany) was performed in the X-Cell Sure Lock™ Electrophoresis Cell (Invitrogen S.r.l.), according to the manufacturer's instructions. The membranes were saturated overnight in 3% non-fat milk in TTBS buffer (500 nM NaCl; 20 mM Tris/Cl, pH 7.5; 0.05% Tween-20) and incubated for 4 hours at room temperature with the human Anti-Mitosin/CenPF ab90 (ABCAM, Cambridge, UK) and/or anti-Alpha Tubulin (Sigma, Missouri USA) mouse monoclonal antibodies. The Anti-Mitosin antibody recognized a weak signal at a very high apparent molecular mass (350-400 Kda) while the Anti-Alpha Tubulin showed a clear signal at 45 KDa. The immunoreactive band was revealed by an alkaline phosphate conjugated affinity-purified monoclonal anti-rabbit mouse IgG (Sigma-Aldrich Inc.) and (in the experiment indicated in FIG. 1C) the ECL detection system (Amersham, UK) or (in the experiment indicated in FIG. 1E) the alkaline phosphatase substrate BCIP/NBT (ICN Biomedicals, Aurora, Ohio, USA).

Anti-21A siRNA Synthesis

The Anti-21A siRNA was synthesized against a region of the 21A transcript of no homology with CENP-F so that the silencing effect was specific for the Pol III regulatory RNA and did not interfere with CENP-F mRNA stability. The siRNA synthesis was carried out taking advantage of the siRNA Constriction Kit (Ambion, USA) according to the manufacturer's protocol. The Sense/Antisense oligos used were: 5′-aaGTGTGGTGGCTCACcctgtctc-3′ (SEQ ID No. 49) and 5′-aaGTGAGCCACCACACcctgtctc-3′(SEQ ID No. 50).

### Results

In Silico Identification of a Novel Set of snRNA Gene-Like Transcriptional Units in the Human Genome

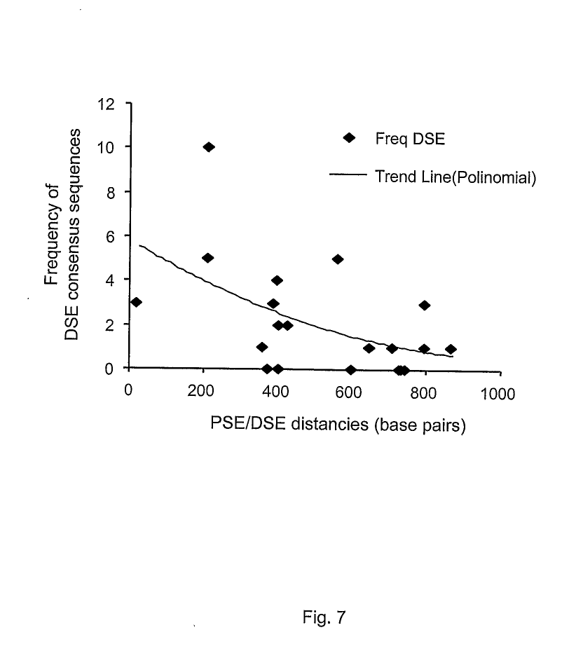

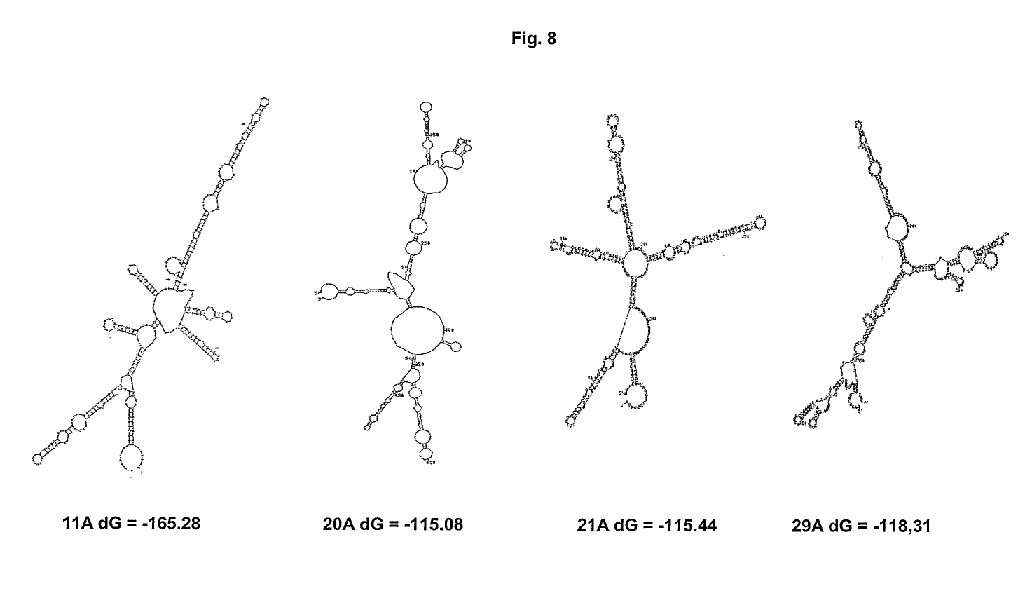

To test our hypothesis we focused on Pol III Type III extragenic promoters, that are located upstream of the transcribed region. We screened the human genome for regions containing the consensus sequences characteristic of Pol III type III promoters: the Proximal Sequence Element (PSE) and the Distal Sequence Element (DSE) [15, 16]. As first we tested the PSE sequences of three well characterized Pol III Type III non-coding (nc) RNAs (U6, H1, 7SL) for their ability to identify a large number of similar (if not equal) elements in the human genome by using the BLAST (Basic Local Alignment of Sequence Tags) algorithm as bioinformatic tool (available at http://www.ncbi.nlm.nih.gov/BLAST; “Short Nearly Exact Matches” option, “Homo sapiens” organism database). (For sequences used as query see Materials and Methods). Interestingly while the first search with U6 and 7SK did not identify a significant number of homologous regions scattered throughout the genome the H1 consensus elements shared a high homology with 60 novel putative consensus sequences. Among these we selected (by a BLAST analysis) those who contained a DSE consensus sequence within an arbitrarily defined distance of 1000 base pairs upstream the PSE. Results evidenced 33 putative novel PSE/DSE-dependent promoters. In order to test the functional relationship between the occurrence of the PSE and the DSE consensus elements within that defined genomic distance we examined the frequency of the DSE consensus elements occurrence versus the PSE-DSE distance in the whole pool of novel promoters. Results pointed out an inverse correlation between the DSE occurrence and its distance to the PSE. A very high frequency of DSE elements was associated to the distance of a nucleosome (about 200 bp) from the PSE that significantly decrease at about 800 base pairs to the PSE [17]. Although the restricted number of putative DSE elements did not permit a proper statistical analysis the inverse correlation between DSE frequency and DSE-PSE distance was taken as preliminary indication of their functional relationship in these novel promoters (FIG. 7).

However, since the Pol III Type III promoters were at the base of our search some of their structural features needed to be considered: i) the occurrence of a PSE consensus sequence does not constitute per se the minimal Pol III Type III promoter that is, on the contrary, the result of the simultaneous occurrence at an appropriate distance of the PSE and an A/T rich element (TATA box). In fact, it has been clearly demonstrated that the occurrence of a PSE consensus that lacks a downstream A/T rich element makes the promoter readable by RNA Pol II such as in the case of snRNA U2 [16]. In this context the transcription start site is not relevant for the choice of the RNA Polymerase at least in humans although it seems to be of fundamental importance in Xenopus [18]. Therefore the putative transcription units identified by our search might thus be transcribed either by Pol II or by Pol III, depending on the occurrence of a functional A/T rich region downstream the PSE. The further occurrence of a TATA box-like consensus sequence downstream the PSE in a large part of the novel element collection further support a canonical Pol III Type III structure pointing toward their Pol III-dependency. Altogether these findings brought to light 33 novel putative transcription units whose promoter organization is compatible with Pol III transcription (Table 1).

TABLE 1
i) The predicted TATA box, PSE and DSE consensus sequences (in sense as well as in antisense configurations) are indicated in bold.
ii) The putative transcribed regions are underlined and arbitrary predicted as starting from the 21^{th} nucleotide starting from the predicted TATA box. A 4×T repeat was considered as stop although events of “read-through” are possible and documented in literature.
iii) The 21A region in Antisense configuration with respect to CenPF mRNA is indicated in italic.
iv) Single strand sequences, complementary strands deducible

Human Genome Map 14q11.2 (784 bp sequence)

(SEQ ID No.51)

11A
ATTTGCATGTCGCTATGTGTTCTGGGAAATCACCATAAACGTGAAATGTC
TTTGGATTTGGGAATCTTATAAGTTCTGTATGAGACCACTTTTTCCCATA
GGGCGGAGGGAAGCTCATCAGTGGGGCCACGAGCT6AGTGCGTCCTGTCA
CTCCACTCCCATGTCCCTTGGGAAGGTCTGAGACTAGGGCCAGAGGCGGC
CCTAACAGGGCTCTCCCTGAGCTTCGGGGAGGTGAGTTCCCAGAGAACGG
GGCTCCGCGCGAGGTCAGACTGGGCAGGAGATGCCGTGGACCCCGCCCTT
CGGGGAGGGGCCCGGCGGATGCCTCCTTTGCCGGAGCTTGGAACAGACTC
ACGGCCAGCGAAGTGAGTTCAATGGCTGAGGTGAGGTACCCCGCAGGGGA
CCTCATAACCCAATTCAGACTACTCTCCTCCGCCCATTTTTGGAAAAAAA
AAAAAAAAAAAAAAACAAAACGAAACCGGGCCGGGCGCGGTGGTTCACGC
CTATAATCCCAGCACTTTGGGAGGCCGAGGCGGGCGGATCACAAGGTCAG
GAGGTCGAGACCATCCAGGCTAACACGGTGAAACCCCCCCCCATCTCTAC
TAAAAAAAAAAAATACAAAAAATTAGCCATTAGCCGGGCGTGGTGGCGGG
CGCCTATAATCCCAGCTACTTGGGAGGCTGAAGCAGAATGGCGTGAACCC
GGGAGGCGGAGCTTGCAGTGAGCCGAGATCGCGCCACTGCATTCCAGCCT
GGGCGACAGAGCGAGTCTCAAAAAAAAAAAAACC
Human Genome Map 2p24.3 (3000 bp sequence)

(SEQ ID No. 52)

12A
TGTATTTTAAATTATGATACATAATGACTATTTAACTTCCAAACAGAATT
CACTCATTTACATTTATGAACATTCTGGGTATAATATCCAGAGGGAATTA
AACCACTATCTCAGAGAGATATCTGCATTCTGATGTTCACTGAAACATTA
TTCACAGTAGCCAAAATACAGAAACAACCTGTCTGTCAACGAATTAATGG
ATAAATAAAAGAGATAAGGAATATATATATACACACACATACACACACAA
GCACACACACACACATACAATGGAAAATTATTCATCCTAAACGGAAATAA
AATTCTGCTATTTACAAGAAGAAGAATGAAACTGGAGGACCTTCTGCTTA
GAGAAATAAGTCAGACATAGAAAGACATATACTGCATGATCTGACTTGTA
TGTGGAATATAAAAAAGTAGAACTCATGAAAATAGAGTAGAAGGGTGGTT
ACCAGAAGTTATGGGGTGGGAGAAATGGAGAGCTATTGGTCCAAGGATGC
ACACTTTGAATCATAAGGAATAAGTTCTGGAGACCTGATGTGCAGTAGGA
TGACTATAGTTAATAATCATGTATTATATGCTTGAAATTTGCTAAGAGAA
TAGATATTCAGTATTCTTACAACACACACAGACACACACACACACAGGTA
TGTCAGGTGATGGATATGTCAATTAGCTTGATTGTGGTGATCATTTTTAT
AATATATACATATATCAAAATAGTATATTTCCAGTTTTTCACTTTTCTTT
TAATTTTTATTATCATATATTTTACTATATAAAATATTTTTAACTAACAT
GATGTCAGTCCAGCCTGACCAACATGGAGAAACCCCATCTCCACTAAAAA
TACAAAATTAGCTGGGCATGGTGGTGCATGCCTGTAATCCCAGCTACTGG
GGAGGCTGAGGCAGGAGAATCATTGGAACCTGGGAGGCGGAGTTTCTGGT
GAGCTGAGATCACACCATTGCACTCCAATCTGGGCAACAAAAGCAAAATT
CTGCCAAAAAAAAAAAATTCTGGACAGAATTTTGCATAGAAAGCCCTTTT
TCATCCCCAAATTATAATAATAACATAGTACATTTTCTTTTTGTAATTCC
AAGGGATCTATTTTTTGTTTATTTTGACATATAGCTCTTAGGTTCTTTTG
GCATTATTTAGTGTGTAAGAGTAAGTAAGGATATATTTTTATAGTTTTCC
AAATAATAGCAAATATCCAGAAATAACTTATAAAACAGGTCATCCTTTCA
CCATAAATGTGAAATGCTACCTTTATCCTATGTATTTGAATATATATACATAT
ATATTCAAGTACATTCTCTCTATATATGTGTGTTTATAATATCATATATA
TACACACACATATGTGTGTGTGTGTGTGTGTGTGTGTGTTACCTCTTTCA
ATTCCATAGTGTTTTAAGTAATCTAATTTTGGCATACTGAAAATACTGAT
AAGAAAAATTCTTATTTTTTCTTTCAAAATTTCCTTTGCATTTATAATAC
ATTAATTTTCCAGATAATCTTTAGAATCAGCTTATCAAACTTTGTTAGAA
GTGTATTTTATGTTAATCGAGACAATACTGGAGCTTGTAGAATAATTTCA
AGAAGAAAGACTCATCTAACATATTTGAGTATTTTCATGCAAGGGCAGAG
TATGTTTCTTCCTTTATTATTCTTTGCTCTCCTAAAGTAAAGATTTATAA
GTGGTTTATAATCCTTTTTTACTTTATATTAAGTTTATCTCTAGTTTTTT
ATAGTTTTTGCTATTATTATGGCTATAATTGTCTTTAATTGCTATTTTTA
ATTGAATGTTAATGTGTTAAAGGAAAACCATTAATTTTTGTATACTGATT
TGTGTCCTGTTAAGTTCATAAACTAGATTACTACTTCTAAAAGTTTTATT
TGATTGTTTTGACCTTTAGGTATAGAAAATCACATTGCTTGCACCTTACT
GCAAGTTCACAAATCCTTTCCATTACTTATACTTTGTAATTTTTTATCTG
TATTTAAATTAAGTAGTCCAGTGCAGTCAATATTGAATAAATGGTACTAG
TTATAGCAGGCCAGTTTTACTTTTATTATGAATTCTTACAGTATTATCAA
AGACGTTCTTTATCAAGTGAGGCAGTTTTTCCTATTTGCAGTTTGTCAAG
AGTACATTTTTAGTTTTACTATAAATTTGCGTTGAGTATTATCAAATGAC
TTCTTTCATATAGTGTCATTATCATGTATTTTCTCCTTTCACATTATCAA
GTAGAGAATTACATCAACAGTTGTCCTAATTCATATCATCCTGAACAAAT
TCTACTTAATCATGGCACACTAAATATGTAATTTTTTATAATAGTTTGGA
ATCTGAATTTATATTTTTGAATAAAATTTGTCGATAGTTTTTATTTTTCT
ATATGTGATTAATTTGCATTTTTGGAACAATGATTTTGCCAGGTTCATAG
ACTGAGTGAGAAAGGTCCATTAAAACTAGATTCATGTATTATTTGCTGAT
TAATAAATTAAATAACTTAGAAGTTATCTGTTAATAAATAGGTTACTAAA
ACTTGCCAGTGAAATTAGAGCTAAATTTTTATTTTTGTGTCAAGTATTTG
CCGAAATTCACTTTCTAAATTGTTATTGAACTATTTAAATTTTCTACCTA
TTCTTGAATCAAATTTTGTAATGTATATTTTTGTCAGTCTTACATGTTGT
TATAATTTTTAATATTATTTCATGCATTTGCATAAAATGTCACAAATTTC
TAAAATACGTTATGTTCCTGTAATTATGCTCATTATTTCATACTAACAAT
TTTCATTCTAGTACTCTTTTTTCCCCTTTCATCAGACTTAACAAAAGAGT
TGTCTATTTTATGAATCTTTGCAAATAAGTAGCCCTTGATTTTTATTTTT
AAGCCTATTTTTTATATGGAATTGTAATTGGAAGATTTAAAAAGTCAATA
TTGCACTGGAAAATATAAAATAAAAATTAATGTTTAATTCTATGTGT
Human Genome Map 3p12 (1921 bp sequence)

(SEQ ID No. 53)

14A
TTCTCTTTTCTCCACATCCTCACCAACATGTTATTTTTTGTCTGTTTAAT
AATAGCCATTCTAACTGATGTATGATTATATCTTATTTTGGTTTTAATTT
GCATTTTTCTGATTAGTAATGTTGAGCATTTTTAATATGCCTCTGGGCTA
TTTATATTTCTTCTTTTAAAAATGTCTATTCATGTTCTTTGCCGACTTTC
TAATGGATGATGGAATGCTAAAGGCCCAGACTTAACCACTATGCAATATA
GCCATGTAACAAAAGTGTACTTGTACCTCTTAAATTTATGCAAATAAAAA
CTCAAAAAAAAAAAAACAAAAAAACCTAAGATGACTAAATGTCAGAAAAC
CAGGTTTTACATGCCACTTCATTTGCTGAAATACAACGTACACAGCCTGT
TAAAATGAAGTCGTCTGCCCCCCAAAATATATAATATTAATAAGGTCTCT
ACCTAGAATCACCAGTTTACAATAAATGCAGAGGATAGATGCACATGTTA
GAAAACACCATAAAGGTGAAATCACCCAAAGTCTACTCGACAAATTATCC
AACTTCTTCAACCATTAAATAGCATAAAAGTTAGGGGAGGGGAATCTGTT
ACAGAACAAAGGAGATAATAATATATCATGCAAACACAAACCCATCTATA
TCTTGATTCAAATATAAATTGCAAAAAACGGCTTGAAATTACTATAGAAA
TTTCAACAGAAACAAGGTCTTAGATAAACAGTCCCCAACTTTTTTGGTAC
CAGGGACCAGTTTTGTGGGAGACAATTTGTCCACAGACAAAGGGTGGAGA
GGTGGGGATGGCTTCAGGATGAAACTGTTCCACCTTAAATCATCAGGCAT
TAGTTAGATTCTCAGAAGGAGTACACAACACAAATCCCTCACATGTGCAG
TTCACAATAGAGTTCATGCTCCTACGAGAATCTAATGCTGCTGCCCATCT
GACAGGAGGTAGAGCTCAGGCGGTAATGCTTGCTTGCCTGCCACTCACCT
CTTGCTGTGTGGCTCCGTTCATAACAGGCCACAGACTGGAACCCATCTGC
AGCCCCAGGGTTGGGGACCCCTGTCCTAGATAACATTTAGTAAATACAGT
TAATTCTTTTCAGTGCAATAACTTTGTTGAGATTGTTTTTTAACTGGCTA
GCCATATACAGAAAACAGAAACTGGACCCCTTCCTTACACCCTATACAAA
AATTAACTAATAAAAAAACACTGACATGTAAGACCTAAAACCATAAAAAC
CCTAGAAGAAAACCTGGGCCATACCATTCAGGACATAGGAAAGGACAAAG
GCTTCATGACAAAAACACCAAAAGCAAAGGCAACAAAAGCCAACATTGAC
AAATGAAATCTAATTAAACTAAAGAGATTCTGCACAGCAAAAGAAACTAT
CATCAGAGTGAACAGGCAACCTACAGAATGGGAGATAATTTTTGCCATAT
ATTCTTCTGACAAAGGGCTAATCTTTGTCAGAATCTACAAGGAACTTAAA
AAAATTTACAAGAAAAAAGCAACCCCATCAATAAGTGGGCAAAGGATGTG
AACAGACACTTCTCAAAAGAAGACATTTATGCAGCCAACAAACAAATGAA
AAAAAGCTCATCATCACAGGTCATTAGAAAAATGCAAATCAAAACTACAA
TGAGATACCATCTCACACCAGTTAGAACGGTGATCATTAAAAACTCAGGA
AACAACAGATGCTGGAGAGGACGTGGAGAAATAGGAACGCTTTTACACTG
CTGGTGGGAGTGTACATTAGTACAACCATTTTGGAAGACAGTGTGGCAAT
TCCTCAAGCATCTAGAACAGAAATACAATTTGACCCAGCCATCCCATTAC
TGGGTATATACCCAAAGGATTATAAACCATTCTACTATAAAGGCACATGC
ACACGTATGTTTATTGTGG
Human Genome Map 9q22-9q31 (2521 bp sequence)

(SEQ ID No. 54)

17A
TTTCAGCCTCCCTTCTACCCCACTCCAGGTACTTCTGCCTCTGTGGAATT
CCTGCTGATTCTAAGCCATGATGAGCATGGCTACCCTACCCTCTGATCTT
CCCTCCTACCGTGCTGGGCTCCTGTAGGAGGGGATCCCTCTCTTCCTCCT
CCACCAAATGTTGTCTCTTTTTGGAACCTTGTCTGAGCACTCTCCCCAGG
TGGGATGAGTCACTTCCTCCCTTTGTTCCCAGGCCCCTTTGTTCCTGTTT
CCCCTGAGAGGTCTCTGTCTTCTTCACCATGCTGGGAGTAACCTGAGGAC
AAGGTCAAGGCCGATGATGTCTATGAGCCCAAGAGAGGGTCTGGTGCGTA
AAAGCTGTTTGAGAGAGTATGCAGAAGGAATGGACAAATGAAAATTAGAG
ACTGACTTACAACTGGGGAAACTTCTCGTTGACCCTTTCTGTTCCTAAAG
AGAGTGTCACCGGATAGGGGTCAGGAGCCTGGGCTTTCAGTTGCAACAAG
AAGACTTCTTTGCTGTGGGCTTTCTGAAAGACAGTTCCTCTCTCTGTGAC
TCTTCAAAACAGACATGACAATCATGTGTGCCCTGCTTGCCCCTGAGGCT
GCGTTGAGAGATATAAAACCATCAGGAAAGTGCTCAGTGGCTGTGCACCT
GCAGCCAGCACCTCTGGCCAGTGTTGGAGAGCAAGGAAGGGAAAGCCAAG
GGAAGCCAATTCCTGGGAGCTTCTCCTGTCTGGGATGCCAAGGTGGAAAT
GAACTTGAGACCCAGACCAAACTTGAGGCTCTTTCATAGTCAGGTAATTT
GGGCACCCAGGGCATTGAGATCAGTCTGCCATTCACCCTGTGGCTAGCCA
CACCTACCTTCAGCTTTTTGACACTGGTACAGGGATCGTTGGAGAAGCTC
TCGGTGTCTGAAATCTCAATGTCCTCGCCATACAGAACTCCAGTCAGGTC
ATTCCGCACCTGTCAGCAAAGAGAAAGCAGAGGGTGGGTGTGCTGGGGAC
CACAGGAAGGGCCAGTTCCGAGGGGTCACCCTGGGGAAGTCAATTGGGCA
AAGCGATTTTCTCTACCGACAATGCAAAGTGAGTGGTTTTGTTTTACATT
ATTAACTAGACCGCCCCACAAAAACTTGAGGATCCCCCAGTCCCACCCTG
CAACTGACACATGGATACAAGGAGGCCAGACAGGGAAGGGACTTTCCAAG
ATTGCCCAGGGAGTTCCTGCAAGAGTCAAGATTAGCACCTTTGCTGGTGT
TTCTCCACCACATCACACTGTCTCCAAATCAGGCTATTCAATTGTGTCTT
TGTTAATATTTTGCACTATTTATTTGCAACATTATTTCACTTTTATGGTG
AGGAAATAGCTAAGATATTCAAAGACAATATAGAGTAAAGGAAAGAGGAA
AGAAGTATGGAACCTGCCTATGATGTTACACGTAACTATGTGTCTACTGA
CACTCAGAATGAGGAATATCTATGGATGTGAAAAGCAAAGGGCTGCAAAC
TCCAGTGTTACCAGGACCAGCCAGTGTGTGAACTAGCCTGTGTGGAAGGA
TATTACAGAATGATAGGGCTGGGCGTGGGCTCACACCTGTAATCCCAGAG
CTTTGGGAGGCCAAGACGGGGGGCTTGCTTGAGCCCAGGAGTTCAAGACT
GCAGTGAGCCGTGATCACGCCACCGTACTCCAGCCTGGGTGACAGAACAA
GACCTTGTCTCAACAGAACAAAACAAAACAAAAGACAGTAATAGTTGGTT
GCTGAGTTAGAATGTGGGTCAAGGGTTACCAGACCTTCTGATTTTTGAGG
GGAGAAATCAGAAATTTAGACTTTTAAAATATAAAATCCCCTAATTTTTA
AATGTTGATACTATTTTGAATTAAAAAAAAAAGAAAGTAAGGATCAAACC
AAATAAACTTAAAGTCTGTATCTGGCCTGTGGCCATGGCTGTGCACCCTC
TGACATATAGCAATGGAAACTGGATTTTGGGTTTTAGTAGCAAGAACTAG
GCTGGGGTTAGGGGATCCAGCTTCCAGGTCCTGCTCTATCACTGACTTGC
TTGTGACCTCAATCTCTCGTGTGATTCTTCCCTTCTCTGGGCCTCAGTTT
CTTCCGCTTTAGGAGATGCTTAAAGCACTTCTTGTTCACACCCATTAGCA
TGGCTATTACCAAAAAGCAAAACCACAAGTGTTGGTGAAGATGTGGAGAA
ACTGGAACCCTTGTGTGTTGCTGGTGGAAATGTAAAATGCTGCCACTGCT
GTGGAAAACAGTATAGCAGCCCCTCAAAAAAATAAATATAAAATTACCAC
ATGATCCAGCAATTCCACTTCTGGGCACGTACCCAAAAGAATTGGAAGCA
GGGACTTGACAGACATTTTACACCCATGTTCATAAGAACATTTGTTCACT
GCAGCTAAAAGGCAGAAGCAGCCCAAACGTTCACTGCTCGATGAATGGAT
AAATGAATTGTGGTGTATACAGACAGTGAAATACTATTCAGCCTTCAAAA
GGAATAAAATTCTGACACAT
Human Genome Map 3p12.3-12.2 (2641 bp sequence)

(SEQ ID No. 55)

19A
ACATATGATTTTTTCAAATTTTCTAAATAGTAATTATTTCCTAGCTCTGC
CTTCTGAAAAGTCCTAGAATTACAACAAGCTGGAAACAATGAACAAATGG
AGCCTTCAGACTGTAATCTCTAAATATGATTTTCCTTTTAGTGAAAAGAT
TTCTTTGGAGAAATAGTTGATTATAGATCTAGGTCAAGACATTTATGAGA
TGACCCTGGGACATTTTATTTTTGTCAGAAAGCCTGGAAAGTATTAATGT
GTCTACACAAAACAAAGGAACCAACTTAAAAGAGCAGTCACTGACCACAG
CAGAGATAATTGAAGCATCAAACTGAATAAAAAATATAACTCATTCAAGC
AGATGCAATTAGTTATCCTGAAAATGATAAATGCATGAATGTAATCAAAC
ATTAATACTTGGTTTCCTGTGCAATTCCTTTTCAGGGTAATGAAAACTGA
TGAGTGAGAGTTAAATGAAGGACTCCAGAAAGAATGACAGTTACAATATA
ATGATTTGTGCCCCCCAAATAAAATAATTGATCTACACAAAATACATCAG
TATTAGGCAAAACTAGATGGTAAAAAAATTTCGAAGAAAAATAGATTACG
GAGAACAAATCAGAACTCACTGATCAAACTTGATATGACTAATTTATAGT
TATTTGAGAAATCCACATGCTGTTTTCCATAGAGGTTGACTATTTTACAT
TCCAACTAATAATGTATAAGGCATTCTCTTTTCTCCACATCCTCACCAAC
ATGTTATTTTTTGTCTGTTTAATAATAGCCATTCTAACTGATGTATGATT
ATATCTTATTTTGGTTTTAATTTGCATTTTTCTGATTAGTAATGTTGAGC
ATTTTTAATATGCCTCTGGGCTATTTATATTTCTTCTTTTAAAAATGTCT
ATTCATGTTCTTTGCCGACTTTCTAATGGATGATGGAATGCTAAAGGCCC
AGACTTAACCACTATGCAATAGCCATGTAACAAAAGTGTACTTGTACCTC
TTAAATTTATGCAAATAAAAACTCAAAAAAAAAAAAACAAAAAAACCTAA
GATGACTAAATGTCAGAAAACCAGGTTTTACATGCCACTTCATTTGCTGA
AATACAACGTACACAGCCTGTTAAAATGAAGTCGTCTGCCCCCCAAAATA
TATAATATTAATAAGGTCTCTACCTAGAATCACCAGTTTACAATAAATGC
AGAGGATAGATGCACATGTTAGAAAACACCATAAAGGTGAAATCACCCAA
AGTCTACTCGACAAATTATCCAACTTCTTCAACCATTAAATAGCATAAAA
GTTAGGGGAGGGGAATCTGTTACAGAACAAAGGAGATAATAATATATCAT
GCAACACAAACCCATCTATATCTTGATTCAAATATCAATTGCAAACGGCT
TGAAATACTATAGAAATTTGAACAGAAACAAGGTCTTAGATAAACAGTCC
CCAACTTTTTTGGTACCAGGGACCAGTTTTGTGGGAGACAATTTGTCCAC
AGACAAAGGGTGGAGAGGTGGGGATGGCTTCAGGATGAAACTGTTCCACC
TTAAATCATCAGGCATTAGTTAGATTCTCAGAAGGAGTACACAACACAAA
TCCCTCACATGTGCAGTTCACAATAGAGTTCATGCTCCTACGAGAATCTA
ATGCTGCTGCCCATCTGACAGGAGGTAGAGCTCAGGCGGTAATGCTTGCT
TGCCTGCCACTCACCTCTTGCTGTGTGGCTCCGTTCATAACAGGCCACAG
ACTGGAACCCATCTGCAGCCCCAGGGTTGGGGACCCCTGTCCTAGATAAC
ATTTAGTAAATACAGTTAATTCTTTTCAGTGCAATAACTTTGTTGAGATT
GTTTTTTAACTGGCTAGCCATATACAGAAAACAGAAACTGGACCCCTTCC
TTACACCCTATACAAAAATTAACTAATAAAAAAACACTGACATGTAAGAC
CTAAAACCATAAAAACCCTAGAAGAAAACCTGGGCCATACCATTCAGGAC
ATAGGAAAGGACAAAGGCTTCATGACAAAAACACCAAAAGCAAAGGCAAC
AAAGCCAACATTGACAAATGAAATCTAATTAACTAAAGAGATTCTGCACA
GCAAAAGAAACTATCATCAGAGTGAACAGGCAACCTACAGAATGGGAGAT
AATTTTTGCCATATATTCTTCTGACAAAGGGCTAATATCCAGAATCTACA
AGGAACTTAAAAAAATTTACAAGAAAAAAGCAACCCCATCAATAAGTGGG
CAAAGGATGTGAACAGACACTTCTCAAAAGAAGACATTTATGCAGCCAAC
AAACAAATGAAAAAAAGCTCATCATCACAGGTCATTAGAAAAATGCAAAT
CAAAACTACAATGAGATACCATCTCACACCAGTTAGAACGGTGATCATTA
AAAACTCAGGAAACAACAGATGCTGGAGAGGACGTGGAGAAATAGGAACG
CTTTTACACTGCTGGTGGGAGTGTACATTAGTACAACCATTTTGGAAGAC
AGTGTGGCAATTCCTCAAGCATCTAGAAGCAGAAATACAATTTGACCCAG
CCATCCCATTACTGGGTATATACCCAAAGGATTATAAACCATTCTACTAT
AAAGGCACATGCACACGTATGTTTATTGTGG
Human Genonie Map 14q22.1 (2341 bp sequence)

(SEQ ID No. 56)

20A
AGGCCTCAGTGTCCTAGACTAGCACAGAACAAGCAGATGAAACAAAGTTT
ATATCAGAATGTCAACTGAAAAAGTATCATTTACCATAAATGGAAGATAA
TTGTATTAAATTCTAGTTAGATGCTAAAACTTCAAGAACTTTTAGAGTCT
GTACCTGCATTCTGTTAAAAATATAGATTAAAAAATGCTAACATGTTAAC
ACAAAGGACTTTCCAGAAAGACTTAAAGAAAAGTGAAGGGGAATAACTGT
CTTGCAATGTAATTCATTGTCGTTTAAGACTGGGTCTATGGAACACCCTA
AATCACCTGGTTCCATCACGTTCTTTTTAACATGGAGATGGATAGTTTTT
CCCCATACTCTATATATTGAGCATTCTATAGTTCATGATTTTTCTGCATA
GAGAATTGTTCAAGCCGGGGGTGCAGGCTCACCGACTGGATAGTGAATCA
AGAAAATAGTGTGTTCATTAGTTCATCATTACCCTGAGTTTCCAACAAGA
ATTTAGTACAGGAAAGTAGACAGCGGAGCTGGGAGCCATCTATTTGAAAC
TGTCTTAAGCAAACTAAGAAACCGAGTAAGCTTGCTTTTGGTGTCTTTCA
TCCCTTCTTGTGTGCCCCCTAATTATTCACTCCCCAATGCCCAGACATTA
TGATGCCTTCTCCTGCTCAGAGACCTTTCTGGGAGGAAGACCTACTCAGA
CCTGGTATTCCCTCATCCTAGGCTCTACCCTATTTTTCATCCAGCTGTTA
AAGCTGAGTGACTAATTTCACACTTATGTACGAATGACCCATAACTGGCT
TAATGCTGTGACCATCTTGGGGGTATTCAAAGCTGATAAACACTTTTTTA
AGTTATATAATAATCAAAGAAGCTTATCTTTCTGCTTTATTTCAAATTTC
ACCCCACAGGCCTTACTTATTTTTAAGATCAATGATTTTGATGGGCCCCC
CCTTCCCACTCTTAATTCAGGGTATTTCTGGCCCCATCCGGATCCAAACT
CTAATGCTCATCTCTTCCATACTGTCCTTTGCAGGTCATCGGTATTGCAA
GAGTTGCATAAGGCCCAATTCAGTCTCTGCCCCAAAAGCTCAAGTCCAAA
CTTCAGAATCTGGGAGGACAAGGATTCAGGAAATTTTGTCAGAACTATGA
CTTTGAACTTTCACTTTTATGGTGAGGGTCACATTTGGTCTGAATCAATT
AATCCATTACCCGCCCCCCCCCCCCCCCCCACCACCACCATGTGTGAATT
CAAAATAATCAACTTGGGTTTATTATAAAAAACAAAATATATTAATATAA
GTATACTAAGATTTTTCTAGAAAACTTGGCCGGGCGCGGTGGCTCACGCC
TGTAATCCCAGCACTTTGGGAGACCGAGGAGGGCGGATCACAAGGTCAGG
AGATCGAGACCATCCTGGCTAACACGGTGAAACCCCATCTGTACTAAAAA
TACAAAAAATTAGCCGGGCGTGGTGGCGGGCGCCTGTAGTCCCAGCTACT
CGGGAGGCTGAGGCGGGAGAATGGCGTGAACCCGGGAGGCGGAGCTTGCA
GTGAGCCCAGATCGCGCCACTGCACTCTGCCTGGGTAACAGAGTGAGACC
CTGTCTCAAAAAACAACAAACAAATAAACTTAGAAGAATATATGTGACTA
TTGGCCGGGCGCGGTGGCTCACGCCTGTAATCCCAGCCCTTTGGGAGGCC
GAGGCGGGCAGATCACGAGGTCAGGAGATAGAGACCATCCTGGCTAACAT
GGTGAAACCCTGTCTCTACTAAAAATAAAAAAATAAAAAATAAAAAATGC
GAGGTGGCGGGCGCCTGTAGTCCCAGCTATTCAGGAGGCTGAAGCAGGAG
AATGGCGTGAACCCGGGAGGCGGAGCTTGCAGTGAGCCGAGATCGCGCCA
CTGCACTCCAGCCTGGGCAACAGAGTGAGACTCCGTCTCAAAAAAAAAAA
AAAAAGAAGAAGAAGAAAGAAAAAGAAAAGAAAAAGGAAAAAGAAAACTT
AATTCTGGCAATGGACTGTTTCTAAAATAATATATTAATACTACTTAATG
AGGAAGAAAAAACCTCTGACATCCTAAAATGCCAAGTGTTTGCCTTTACC
AAGGTTTAAGCACACATAAACACGCATATTCAAATACCACCCAAAGTGGA
GGTGCAAAGATCAGCCTGTACCGCACAGTAACACAGACTGGGTTGTTTTT
TGTAAAGAAGGCAACTAGTCCAGTGAGTAATCCCTTCATTTTCCACACAC
ATACCCTTCTGTTTTCTCCCTCTCCTCCCCCCACACCCTCCACTGCAGTT
AAAACGTAATTCGAAGAAGCCTAAGGTAAAAGCCCCT
Human Genome Map 8q24.13 (2100 bp sequence)

(SEQ ID No. 57)

21A
GTGGACAGGGAAATCTTACCTTCCTGCCTCTCTATGTTCAGGCTGAGTGG
GTCAGAAGGAGAGTGTATTAGGTAAGAAAATTTATCAGTATTATTTAGTG
AACACTGGATTTATCCTTTTGCATTCTGGCTGTAGTACCCAACTTCCACA
TGGCAATGCACCCTCACCTCAGCCCTCCGCCCACGTGGTCCCCTTGCTGA
GCACTTTAATGAATGACTGCATCTCATTTTCACAGCTATTTGATGCACCT
GCTATTATTACTCTTATTACCATTTTCCAGTGGGAAGCTGCTTCTTGGGC
AGGGTGGATTTCCATCTGCGTCTCCTTTTCGGTGTTGAAAGCTGGTAAGT
GAGGACACCAGGATTGGAACCTGGGTAGTCTGAGTCCAGAATCTCTATTT
TCAAGTCTTCCTGCTCTCTGCTTCTGGCAAGTTTGATGTCCACTTTTGAT
CTTCACCTACATTCCAGCATAATAGCTACTTTTGGTTGTTTTCTCAGCAG
CACAAGAGAAGTGTGGCGAGATTTTTAGGTGAGTCATCTAGAGAAGTTAA
TCTTATTTTGGGAATTCTACTGGCAGCTTCAGGTGGGGAAAATTTTGTTA
TTTTCTATCCTCCTCTAGGTTCTAAAAGGGAAGAAAGATGGTGAGCGTAG
AAAGATGTGACTGTATTCACTATTCACCCTTTGTCGGGTGGTGAGTAAGC
AGCTTGCAAAGATGCAATGAAGTTTGGAACAATCCAGAGAACCAAACTTT
CAGCTGCCAGAGATGGCACCTGGTATCCTGGGTACATCTGCCTGTAGGGC
CCAGAAAGAGCTGGAAGCCAAGTGCATGGATCAGGTCTGTAGGAAGGTGG
GAGAGCCAGGAATCGAGTGTCAGGGGGCATTTATTACCCATGGAAGCAGG
TTTTTGTCAATTTTGTTCACTGCTGGATCACTAACACCTGGACTGGTGCC
TGGCCAGGTGGTGGCTTCATAATCATTTGTTGAGTGAATCAATGAATGAA
TGAATGAACAGCTGTAGCAGATGCTAGCAGGGCTTCCTATTTCTTCCATC
ACCATAAAGGTGAAAGACATCATAAACGGGAATTTAGACAATCCTCAGAA
ATTTTCAACTGCCATGTATCTTGACTTGATGCTTCTAGTAGTTATATTTA
TTTGTAATTCAATCTTTCTTTTTAAATAGTTGACCAAGTGTGGTGGCTCA
CGTAGTCCCAGCACTTTGGGAGGCTGAGGCAGGAGGATCACTTGAGCCCA
GGAATTTGAGACCAGCTTGGGCAACATAGTGAGACCTCATCTCTTAAAAA
AAAAAATTAGCTGGGTGTGGTAGTGCACACCTGTGGTCCCAGCTACTTTA
GAGGCTGAGGTAGAGGATTGCTTGAGCCTGGGAAGTTGGGGCTGTAGTGA
GCTTTGATTGCATCACTGCACTCCAGCCTGGGTGACAGAGCAAGACCCTG
TCTCTAAAAAATTAAATAAATAATAAAAAAATTAAAAAGTAACTCCCTTT
TCTTTATTTTCAGGCTTCCTTCCCACCTGCTAATTCAAACACTTTACAAC
CAAAAATATCTTACCTTGATCCTGTTTCTTTCTCTATAACCTCTCTATTT
CTGTTTCTTTCAACCAAATTTCTTAGGTCATCTATAATTTTGTTTCTACT
TTTTCTATGCATGCCTCAATCCATTGCCAACTCCTCAACCTGCCCCAAGT
GCCCACAACTCCACCAAAAGTAATTCTAACATTTTACCAATCCAATACAT
CACAGTTTTTTATAAAAAACTTAAGAAATATACTTTAGTTGAATTTGAAA
GAGTTGCCCACTTGTGTTAAATATTTCTTTCCTTGTGTCTGGGATATCAT
TTGATTCTGATTCTATTCCTAATTCTCTGACCACCCTTTCTTCGTAGATT
TCTCTTCCTTTGTTCAGCCTTTCACATCCTTGGAGTTCCATCCTCTGGTG
ATTGTTTGTCCTGTTCCATACATTCTCCTTATATGAGCATTGTGTTTTAG
CTTATGAATGGTCACATGACCTAGCCAGGCCAATCAGAGTCTTCCATGAG
ACTTTTGTTTATTTATTTATTTATTAATTTATTTATTCTTCCACATGCCA
TC
Human Genome Map 6q16-q21 (2100 bp sequence)

(SEQ ID No. 58)

22A
GCACACCTGAGCAAGGGAGAGGAAAGGGTTCTTATTCCTGACACAGGTAG
CCCCTACTGTTGTGTTGTTCCCCTGTTGGCTAGGGCTGGAACGCACAGTC
AAAGCTAATTCCGATTGGCTATTTTAAAGAGAGCAGGCGTAGGAGCCAGA
ATGGTGGGGCGAGTAGTTTGGCGGGAAGGTCAGTTACAGAACAGGTGACT
CAGGATGACTCAGGTCAGAGCAGGTGACCAGGGGTGTCTCAGGATGGAGC
AGGTGACCAGGGGTGACACAGGATGGAGCAGGTGATAGAGGCTAGGAGGG
GGTTGTTTACTGAAACTAGGGGCAAGGAGATGACGAGAACGAGAAAGTTA
AACTTTAAAATGAAGAACAAAGAACAGGGGAGCTGAACATACTGATAGAA
CTCTTTCAAGTCTACTTAGGTAACTATTTGTTTGTTTTTCTGCTTCTAAA
ATTTTGTTGAAATTTTCTCCTTTCTTATTCTCATTGTTCTTGAGGTTTCG
TGTATTTAAAAAATCTTCTTACTCTGTAATTGTCATAGTTGAGTAGGGAG
CAACGTTAGATTAATATATTCAATACTTCACTGTTACCTGGAATAAGAGC
CCTCTCTTTAAACAAAATATTATGCAGAAATCTAATACAGGAAGCAAATA
AAAACTAGAACTACTCTGGTTCAAATAGAGTGAAGACAGAGCAGATCTTG
TTCTTGTAATTGAAAGGAATATGATATAATAAGTATTGACAATATTTTCT
TCTCACCAAATAAGTTTCTAATTCTATATATAAAGGAAATACTTTCAGAA
TAAAACGAATATATGAGTTTTATTTTTAAATCACAAAACGAAGTTCAAGA
ACATTTTTGAAACTGGGAAGATTCATATTTTAGTATCTGTCAAATGATGA
TAAATTCGGAAGCCAGTGTAATTTATACCCTAGGGGCTGAGGTCTAATTC
AACATATTCCAGTTTCTATTTTCTAAAGCTAAAGAAACATGTGTTACAAT
GTAGATAGGGAATACTTTCTTAATGAACCATGCTGAACTGTAAGATTTTT
AAGACTCCTTTTTAATGCATTACATTACACTGTATCTTGTTTTCACATTT
ATGGTGAGGTTAATATAAAGAGACATTAAACAAATATATTTCTGCTCTTT
ACAAAGGATGATTATTGTTTTCTTACATTTCAACTAAAAATTTCTATAAT
ATTATACTTGCAAGAAGTATAACACTCTTAATGAGCAATACAGTTAACCT
TAAGGTTAACTTGCAAAATTTCATGTCTAATTTAGTATCATTAACACATT
GAAAAATCTCTCCTAAATTTCACTCATCTTGATCAAAATCCATGTTAAAG
GTTTTGAAACTACACTTAATACATCTGCCTTATTTTATGCCCCCACTTAT
ACTACTAGTTATTAATGCACTTTGGACAGCTGGTTCCTCTGCCTTTTGAG
GATTCTGTGGTGTAACTGATTGGTTCTCAGCTTTTTCCACTGCCCATTTG
GGATGCAACCCTTTCAAGTCTGCTTAGGTAACTATAATTTGTTCATGTGT
TTTTCTACTTCTAAAATTTGGCTGACATTTTCTCCTTATTCTTGTTGTTC
TTGAGGTTTTATGCACTTAAAAAATCTTTCTACTGTAATTATCGTAGTTG
AGTAGGGAGCAACATTAGATTCATGTATTCAATACTTCACTGTTACCTGG
AATAAGAGCCTTTTTTAAGGGCTCTTACTGAAAAACACAATACACTTATG
TTCTTCTATAATGTTTTAAGGAATTTTTTAACATTAATCTCCTGTCTCAG
CCTTTAAGGCCATTAAATGACTTAAGATAGTTGCTGTGGCTCCAAACATT
GTATCCACATTTCCACAAGAGGAAGTAAAAAAAGAAAAAAATGAGTCTTG
CCCTTTCCTTGTAAAGACAATTTCCACAAGGTTCACATTCCTCTGGTCAG
AAATCAGTCATACACCCACGCCTAGCTGGAAGGTAGGTTGGGTAATGTGG
ACTTTAATTCAGACAGTATTGTGTCAGTAAAAGTCAGGGGTTCTATTATT
AGAAGCAGTGTAAACAGACATGAGAAAACAAACTGTAGCCTCTGTGCTT
Human Genome Map Xq21.3 (2160 bp sequence)

(SEQ ID No. 59)

23A
TAAATATAAATAAATCATCACTAGGTATGTTCTAATAAAATTTCAGAACA
CCAAAGATGAAGATAAATTTTAAAAGTAGCCAGAGGAAAAAGGTGAATTA
CCTTTAAAAGGTTACAGTTTAACAAGAAGTCTGACTTTTCAACTACAAAG
ATTTTCAGAATAATATCTTCAGTATGCTGAATGAAGTTTTAAAAAGCAGC
TGCCAAAAGAGTTTTAAACTGTGTAGGTATATTTCAAGACTAAAGGGAAA
TGAAAACATCTGTAGATAATGAAATCTGTAATTACCAACTGAACTTAAGG
ATATGCTTCAAGTGGAAGAAAAGTTAATTGAAATGGAGGGTCTGAGATGT
AAGACAGAATGAGAAATAAATAAAATAGCAAATATATAAATAAATCTAGA
TGAACACTGACTGCAAAAAGCTACAATAATGATAACATCTTTGGGCATTT
AAAAAGATAATTAAAATAAGTAAGAGGAGTGACAAATTCTTTGGGAGGTG
ATTAAATTGAATGAAAGTATTCTAAGGTACTTGCACAGTCACAAGGGGTG
AAAAAAGGTTTTGTTTATAGTAAGACTTTGTCAAGTAGGCATATGTAATT
TCCAGGTTACCCATAAAAAATTCAAAACAGAAAGTGAAATTTCCAAATTA
GTAGAGAAAAAAGTGACATAATAAAAATTATTCAATTCAAAATGAGGCAA
AAAAGAAGAGACAAAGAAACACGGGATATTTAGTACAAATAAAAATCACA
ACATAATACAGTTCATTAAAACTCAAATTTGTTAGTAATTCCATTGATTA
TATATGAATTATGTGTTCCATTTATGAAGCAAAATATCAGAAGAAAAATC
CACCCATATACTGTTTTAAAGAAACATTTAAGACATAAGAATACAGAAAG
TTTGGCAGTAAATGAATCAAAAATGAAATAATGAGTCTACGTGAGGATAA
TCAATGTTCACTGAAATATTAGGGTGAAATGCTGATGGAGAACTTTATAA
TAGATATACCAGGTCTATAACACCTGAGCCTAATCAAATGTAACACCATA
AAAGTGAAATATCCAGACGCTATCCACCAGATTATAGGAAATGCAAAGCA
AAAGAATATTAAAAGACATAAAGATGACTCAATCTTGCAATTCCAGAACA
TGGGAACTTCTAAAAGATAAATTGTTTTAATCAATATGTAGTAAAAAGGG
AAAGGGAACTGTTATTGAATAAAAGTGACATCGTGACCAAATGTAATGTA
ATAACTTTGGACACTGCTTGAAGAAACCAACTATAAAAATTCATATTGAG
TCAGTCAAGAACATGTTTATATTGACTGGAATTTTATTACTTTAAGGATT
AGTATTAATTTTTCAGTGTAGTAATGGATTGTAGTTATAGTAAAAAAAAG
TTCTTATTTTTGAAATTTACATTGAATTATTGATGAATAAAATTATGTGA
TATTTGGAATTTTCTTTAACATAATTTTCATTATTAATAATAAAATCATG
AAAAGGAACAACTCTTGTTGAATGCACATTGGAACTCTGTTGAAGCAGGC
ATTTCTGACCTAGGGGGAAAAAAAACATAAAAGAGAAGATTTTTATGTGA
TAAATACAGGTGGTTGCCAGGGGCTGCCGGGTGGGGAAAATGGGGAGATG
TTAGTCAAATGGTACAAAGTTTCAGTTGTGCAGGATGAGTAAATAAGCTC
TGGAGATCCAGTGTACAACATGATGACTATAGTTAATAATACTGTATTAT
ATACTTAAAATTTTCTGAGTAGGTTTGAAACGTTCTCGCCATACACACAC
AGAAAAGGGTAACTGTGAGGTGATGAATACGTATTCAAGCTAATCACGTA
ATTAGCTCGATTGTGGTATTTATTTCACAATGTATAAGTAAATTAATAAA
TCACATTGTACTCAACTATAWATATTTTTTGTCAATTATACCTCAATAAA
GCTGGGGAAAATGTAAAATAAATAAATAAATTACCGAAAAJ4ACCCAAAC
ATCCATAAATGAAAATGATACCAAATCTGGCGCCACTTTTTACAATGGAT
GTAAAAGTCAAGAGTTAAAATCTTTAACATGCATGCTTACTATGTCGAAA
GATCACGTACATGAAAACAAACATACTTTATTGTGATTTTTTTGAATGTA
AGCGATGAA
Human Genome Map 12q21 (2219 bp sequence)

(SEQ ID No. 60)

24A
TATAATTATTAACTGAAGTCATAGTTTACATTAAGGCTTACACTTTGTGT
TGAATAGTTCTATGGATGAGGGAAGGGGCTAAAATGCATAATTTTATGCA
TTCACCATTAAATATCATGGGGAATAGTTTTACTGTCTTAAAAATTTCCT
TCATTTCAATTATTTGTTCTTCTCTCCACTCTCTAAAGCCCTGGAAACCA
CTTATCATTTTATTGTCTCTATATTTCTGTCTTTTTCAGAGCGTCATGTA
GCTGGACTTATACAGCAAGTAGCCTCTTCAGATTGGCTTCTTTAACTTAG
TAATATTCATGTAACATTGCTCCATGTGTTTTCGTGGCTTAATAGGTCAT
TCCTTTTCATTACTGATCATTTTATTCTGTGCATGTACCACAATTTGTTC
GTCTACTACTGAATGATGTCTTGATTGTTTCGGTTGTTGGTGATTATGAA
TAAACTTGCTATAAACATTTACTTGTGTGGATGTAAGTTTTCAACTTATT
CAGATAATATTTAAAAGAGCAATTGCTGTATAGTATGGTAAGATTATGTT
TAGCCTTGTATGGAACTGCCAAAGTGGCTGTACCATTTTGTATTCCTACC
AGCAATGAATGAAAACACCTGTTGATCTGCATCCTTACCACTATATGATA
TTGTCATATTTCAGATTTTAATCCGTCTAATAGATGTGTAGTGGTAGATA
GTTGCTTAATTTTCAATTCTCTTATGACATACAATGTTTAACATCTTTTT
ATATGTATATTTGCTATCTGTATATCCTCTTTGGTGAGGTGTCTGTTCAG
ATCTTTTTCCCATTTTAAATTGGATTGTTTTCTTATTTTTGAGTTTTAAG
TGTTCTTTTTATATTTTAAGTGCAAGCCCTTTATCAGATATGTATTTTGT
GCATATTTTCCCACTCTGTGGCTTGTATTTTAATTCTCTTAATAATATCT
TTGCAGAAGTTTTTAATTTGAACAAATTTCACTTTTATGGTGTGCTTAAG
AAGTTGTATCTAAAAACACAAGGTCACCTATATTTTCTCCTGTTACAGAA
GTTTTAGACTGTGGGTTTTTTATTTAGCTCTATGATCCATTTTGAGCTAA
TTTTTGTGAACTGTGTAAAGTCTATGTCTGGATTCTTTTTTTTTCCAATG
TAGATATCCAGTTGTTCCAGCATCACTTGTTGAAAAGATTATCTTTTCTA
CAGTGAATTGCATTTGTTTCTTTGTCTAAGATCAGTTTACTATATTTGTG
TGGGTCTATTTCTAGGCTCTCTATTCTGTTCTATTGGTCTATGTGTTAAT
TCTTCCATAACATGCTGTTTTGACTATTGCAGCTTTATAGTAAATTTTCC
ATTTGAATTGTGTCATATTCTTCTTTGTTCTTCTTCTTGTGTATTATGTT
GCCTATTCTGAGTCTTTTTGTATTTTAATATAAACTTTCTTGTCAATTTG
TTGATACACAGAAATAACTTGCTTGGATTTTAATGGGAGTTGCAATGAAT
GTGGAAATTAAGTTGAGAAGAATTGACATCTTAGCAATATAGAGTCTTTC
CTGTCCCTATACATAGAATATCTATCTAGATCTTCTTTGATCTCCTTCAT
CAGACTTTTGTAGTTTTAGCCACATAGATCCTGTACATATTTTGTTTGAT
CTATACTTAAATATTTTATGTAATCAATTGACTTTTGTATATTAACTTTT
TATCCTACAACCTTGCTATAACAGCTTATTAGTTTCAGGGACTTTTTGCC
AACTATGGGATTTTCTGCATATAAATCATGCAAAATATGCAATCATGTCA
TCACCAAACAAATATAGTTCTATCTATGCCTTCCCAATATGTACACCCTT
TATTTCTTTTTCTTGTCTTATTGCATTGGCCAGGCCTTCCAGTACAATGT
TGAAAAGGAATGGTGAGATACAATATTCTTGCCTCTTTTTTCATTTTACG
AGGAAAGCATTCCTTTTAATAGTAGGCAGTCAGAATATAATATGTAATAT
TTTTAAAGGCAATAAATAGACATCTAAGTGAGTTATTTTAAAATTGAGAG
TTTAAAATCAAATAAAACTAAAAGAATTTAATAGTTTGCCTCTGTATCAT
GGAATGAAGGAGTTAACAAGAGCTGATGAGAGAATCTGCTATTTGTCACT
ATATCTTTTATTAGCATTTGACTTTTAAATATGTTACAATGAATATTTTA
ATAATTTTCTTCATAA
Human Genome Map 7q22 (2160 bp sequence)

(SEQ ID No. 61)

27A
ACATCTGTCTGTTTTGTGTGGCCGTCACAAAATAATCAAAGACTAAGTAA
TTTATAAAGAACAAAAATTTATTTCTCATAGTTAATGGAGGCTGAGAAGT
CCAAGACGAAAGTGCTGGCATCTCATGAGGGTCTTCTTCCTGTGTCCCCA
CAGGGCATAAGAGTGTAAGACTATGAACTCATTTCTGCAAGCCCTTTATA
CAATGATGTTAATTCATTCATGAGAGTGGGGACCTCGTGACCTAAACACC
TCCCATTAGATTCTACCTCCCAACACTGTTGCACTGTGCATTGAGTTTCT
AACACATAAATTTTGGGGGGCACATTCAAACCATAACATGGAGTTTTCTG
CATTGAGAAATGAAGGATCCATTTAATACAGGGACCTCAAAATACAAAGA
GAAAACTGACTGGCTGTATGGAGCTAGACGAAGAGGAGTAAGAAACTACT
ATTTGCAAGGCTGTGTAATTCCAAGGACTGTTATTCTTGGATGCTATGAT
GTTTTTAAAGAACAJACTACTATACATTTGTAAGTTATTAAATTATTAAT
ATTATTTGAGAATTTCAAATGGCTTAACTAATCAATGTGACATAGTGGGA
AAATTGGGCCTTTAATTGAAGACACAATTTGCTGATTACCACTTGGTAAC
TTAGCCCTTGCTTCTCTAACCCTTAGTTCATCTTGTAAAATGCATTAGTT
CTACTCCATAGGGGTATTGTGAGATTTAAACGCAGTGCAGCATATTAAGC
ACCCAGTGTAGTCCCTGATACATAGTGAAACATCAATAATAAATTGTTGC
TACTGGTAGAAATCCCTTGGCGTTTGGTAGATTTCCAATAAATACTAATT
CTTCTAAAACTTTTAATGATTATGTAGATAGATATATGCCTAGATCTGGT
AACAAATATGCTATATCAATAGTCAAAACATTCTCTCTTAATTTTATTAT
GATATATATTGGAAATCTTAGTGTGGTTTTGATTATACTAACATAATTAT
GTGGCATTAGTATGCCAAATGTACTCACAGTTATGCCAAAATTACCTGCC
CCAAATTACAGCTAATCCTTTCTTTGGTCCTAGGAGAGATACGCACACTA
GGGATATCACCATAAAAGTGAAGAAACACTTTATTACTGGCTGGGCTTGT
TTCTGAAATTCTAACACAGAGTTCTTATAACATGGACTTTTCCTTGCCTC
CTAGTTCAAGCTTGAGGGCTTACTGTGCTCTTGCAGGGAAAGATAAAAGA
AAGTGTCAGAGTGAAAGAATGGTCAAATGTATGAACTCTTCTTTTATTTA
TTAATTTAAATACAGTGACTCTGTTCACTAGTAAACACACCTAACCCCTG
CCTTAGAGTCAGATTAACATCTTCTTTGAGGACAGCCCAAAGAAGAAAAA
TGCAAGGATGAAGCCTAGAGAGGTTTCCATCTCGTATACTTATATTCCAC
TATCTTTGGTTCTTTCTTTCAACCATTAGACTTAAACCCAACTGTATAAT
TAATCAAACATGTGGATATTTCCTTGGAGGAAGAAATAGAGAAGTGTCAG
GGAAGTTCGACCGCCACCTAAGTGTGTCTGCTTTTTTAATGCTGCCTTAT
GGTCTAAAGAGATGGGTGAAAAGCAGAGTATTCATTTCAAGGCCATACTA
TATTATATGCCATCTATTCACTCCAGGCTGCTTGTTGTCAAGGAAGAATA
AAAACCTTGATATCAAAGAGAATTAAGCTCTCAAAATTAGTTTCTCTTTC
ACATACCAAAGTAACCTTGAGCTTTCTAGCCTGCAAATTTCTCTCCCTTA
ATATTCTTTCTCTGTTCCGTTCCACTGAAAGTGATGTCACAGTGGTGTAG
TTAGAGTCTGGGTTACTCTTTCCTGCAGAACTGTTCTTCAGTACCTCTAG
ATAGAAAATAGTCCAACATCAAGTCTTGCATGAGTTTTCCTTTACCAAAG
ATCTAGTAGTCTAGAAGATATTTAAAAATCACATTATTGAGCCCACATCT
GCAAAAAGGAGAAGTATATACAATATTCTTAGGACTCAATACATATTACT
TGTGTGCTTGTCTTTGCATGGACATGTATGTGTTTTAATTTCTCTTGCGT
AAACACTTAGGGTTGGCATTGCTGACCCACATGGTAAGTGTATGTTTAAC
TTTATAAGCA
Human genome Map 11p15 (2160 bp sequence)

(SEQ ID No. 62)

29A
TACTGCTTCATCCTTGAGTTCTACAAAACACACACACACAAAAACCAACA
AAACTTAACTATAGGCTGGGTGAAGTGGCTCATGCCTGTAATCCCAGTGC
TGTGGGAAGATCTTTTGAAGCTAGGAGGTTTAATCAGCCTGGGCATCAAG
GCAAGACCCCATCTCTACAAAAAAAAAAAAAAGCCAGGCATGGTAGTGCA
CACCTGCAGTCCTAGCTACTCAGAAGGCTGAGGTAGGAGGATCACTTGAA
CCCAGCAGTTTGAGATTGCAGTAAGCCATGATCACATTACTGCACTCCAG
CCTGGCTGACAGAACAAAACACCACCTCTAAAAATAAAAATATAAAATAA
ATAAAAAAATTTAAAAACCTAAACATAGCTGCACTTTACTCAATATATTT
ACAGTTCTACATATGTAAAAACTTGTATATTGACTATGTTTTAAATGTGT
AGGGGAAGTTTCTCACCTAAAGGAGTCCCATAGTGAACATTTAAGAGCAA
ATGATTCCTTTTTTATTTGTATTTTTGGTTTTGCCTCTAGCACATCAGGT
ATTCTTTAAGAAGGCTATGCCTCTGAGGTTGCATGATCATTAACTAATTC
ATAATTTCCCTTGCATATATTTGGGTATTTTGGTGTTTCAGCCTTTCCCA
CACTTTTTTTATTTGCATGTCTTCACGATCACCATTATATCTTTGTTCCA
CCTGTACTATTATTTACTCACTCTTTGTCTTTAAATCAAATCACGTTTCT
TACTCAAGTAGATTTAGTTTTAAGACAAACCTTATGGCCGGGCACAGTGG
CTCACACCTGTAATCCCAGCACTTTGGGAGGCCAAGGCGGGTGGATCATG
AGGTCAGGAGTTTGAGACCAGCCTGGCCAACGTAATGAAACCCCGTCCCT
ACTAAAAATACAAAAAATTAGCTGGGCGTGGTGGCGGGCACCTGTAATCC
CAGCTACTTGGGAGGCTGAGGCAGGAGAATCACTTGAACCCGGGGGGGCA
GAGGTTGCAGTGAGCCGAGATCGTGCCACTGCACTCCAGCCCAGGCAACA
ACGCGAGACTCTGTCTCAAAAAAAGAAAAAAAAGGAACTTTATGTCGCTA
CCATAAATGTGAAATTACTAGAACTCACAATAAATAGAAGTTAGTAAAGA
CACTGAATTCTAACTAGACGCTATTGCTTGTTGAAGGCTTTGATCTTAGG
AGGATTAGAAAGCATTCTAGGCCAGGCACGGTGGCTTCCTGTGTGTAATC
CCAGCAGTTGGAGAGGCTGAGGCAGGCGGGTTGCTTGAGCTCAGGAATTT
GAGACCAGCCTGGGCAACATGGCAAGACCCTGTCTCTACAAAAACATACA
AAACTTAGCCAGGCGTGGTGATGGCCACGTATGGTCCCAGCTACTCAGGT
GGCTGAGGCAGGAGGATTGATGAACCTGGGAGGCTAAGGCTCTAGTGAGC
CATGATCACACCACTGCACTCCAGCCTGGGTGACAGAGCCACACCCTGTC
TCAAAGGAAAAAAAAAAAAAAAAAAGAATTCTAGTGGTGTGGTGTGGAAG
ACACATTCTCAGCAGACTAAGGTTGTATCTTTATAACCACAAGGATTGAA
AAAGAACGGAAGGACAATAACTTTCTCATAAGGTGATTCAATGTTATTTA
GTGCTGTTTCTGTGTACCATCAAAAATCCTCTTACTACACACAGAATATT
ATAACACCATCTCATTGTCCACATGAGCTCAGAAATTGGTCATCAAAGCA
GAAAAGTCTTTAAAACATTGATCTCCGGCCGGGCGTGGTGGCTCACACCT
GTAATCCCAGCACTTTGGGAGGCTGAGGCGGGCGGATCACAAGGTCAAGA
GATCGAGACCATCCTGGCCAACATGGTGATCCCATCTCTACTAAAAATAC
AAAAATTAGCTGGGTGCAGTGGCAGACGCCTGTAATCCCAAGCTACTCGG
GAGGCTGAGGCAGGAGAATTGCTTGAACCCAGGAGGCAGAAGTTGCAGTG
AGCCGAGATCACGCCACTGCACTCCAGCCTGGGCAACAGAGCCAGACTCC
ATCTCGAAAAATAAAATAAAATAAAACATTGATCTCCAAGAAAGTAGATC
ATATCTGCTCTCTATCTGACCACATTGTTAAACTTGGTTATGTTTGCAGG
TTAAAG
Human Genome Map Xp21 (2040 bp sequence)

(SEQ ID No. 63)

30A
CAAAGACAGGCCAGTGCTCTACTCCTTGCTTCCTGGGCTCCCCAAAAGGG
AGCTGACTCCTCATCCCTCAACCTGGAGAACCAGTTCAGCTCTTCTTTTC
ACCAGAATCCTTTCCCTGCTTCCGACTCATCTTCTTTTTCTCAAAGCTGT
TGTAACTGTATTGTTCTCACCTGCTTTGCCCAGACAGATTCCCCAGTCCT
CCCCATCAGTGTTTGGCATTTATTCTGGGTGTTCTACTAGTAATGCCCAG
CCCCGGTCCTGGGCTTCCTGCTGTTTCTATTGCATCTCCCTAACTCTTAC
ATCCACCCCAACTCAGTGTTTTTGGCCTTCCTCAGCAACCAGGAATCTAA
ACCACCCTCCACCCCATAGCACCCTATGGATGACGGAGCCTTAGTTCTTG
ATGGTGATGCAGACACCTTGAGGTGTGGCCATGACATTCACTCAGCCCTT
GGCCTGGTAGCAGCAATTTTCCCTGATAAGGTCCCCAAACTGACCCTCAG
TTGTCCCCTGCAGTCCCATTAGGGCCTGTGGAATTTACGACTTCCATACA
CAGCACCAGGAAGTTGAGGATGGCTCCACGTGCTAGCTCAGTCTCTTTGC
CCTCTCTCTGCCTGTGGCAGATTGTATTTTCCAAAGATGACTGCACCAAA
ATATTCCACCCCATGTTATCTTCTTATGTGAAGTTCACACTAATTCTTCA
AGAAATGGGGCCTCTGTTTACACCTGCTGAATCTTGGCAGGCCTATAATT
ATAGTGGTTATGATTCTAGTGATGCTATATGACTTCTGAGACCATAAAAA
GACAATACAGCTTCCACCTGGTCCTATTGGAACAGTCATTCTTGGAACCA
AGCCACCATGTTGTGAGAAAACCCAGCCCACATGGGAAGGTCACATGTAG
GGATGACAGTCCCCACTGAGCCCCAGCCAATAGCCGGCATCAACTGCAAG
ACATGTGAGTAAGCGAACCCTCAGATGATTCCAGCCCCCAGCCTTTGAGC
TGCCCCAACTGATGCTTTGTGGAACAGAGAAAAGCTGTCCCCATTGAGCT
CTGCTCAGATTTCACATTTATGGTAAAAATCTATATGGTCCTTACTTTAA
GTTACTAAATTTGGGGATGCTTTCTTTACATAGCAGTAGGTAATTAGAAC
ACTGCCTGATCAAACTGCACTGCAACTTTTACTCGGCTGCTAACTATATG
GCTATAGCCGAGCATCATGGGGCCACCGTGTCTGGCAGTCCCCACATCCG
AGTTCCAAATGCGGAGCACAAAAGTCCCACTGTCACTGATCTTCCCTTCC
ACTCTCAGAATCTCAGTCTAGTATGGGGAAGCAAGGGTCGAACCATGTGC
TTCCCCCGTCAGGGCAGATGGTTCTCTTCCTGCCTGGAAGGAATTCCCTC
TACATAAAAGCCTCTTTCCACCAGGTATGGTGGCTCAAGCCTGTAATCCC
AGCACTTTGGGAGGTGAAGTGGGCAGATCACCTGAGGTCAGGAGTTTGAG
ACCAGCCTGGCCAACATGGTGAAACCCCATCTCTACTAAAAATATAAAAA
TTAGCTGGGCATGGTGGTGGGCGCCTGTAATTCCAGCTACTCGGGAAGCT
GAGGCAGGAGAATCACTTGAACTCGGGAGGCGGAGGTTGCCGTGAGCCGA
GATCATGCCACTGCACTCCAGCCTGGGCAACAGAGTGGGACTCCATCTCA
AAAAAGTAATAATAACAAAAAATTTTTAAAAAGTGTTCTTCTTCCCAAGA
AAGCAGACATCAGACATCTTTCCCCCTTCATTGGGGCCTTAATTGCAGAT
GGGACTCTGGAAGAGATACTGACATAAGCATAAAAGTAGGTCCAAGAATA
TTTAACCTCTACATAAGAATTCAAATAAGCTATTGACCTTATGAGAGAGT
CACAATGATGGACACCTTCAAAGGAAGGAGAAGCACCATGGAGGGCAGAG
GAGAAAGACCATACCTGCTTGACTTGGTGTGAGAGGCATTCTAGATGTTA
TTGACATCATATAATAGACAGATGACAAGCATAGAAG
Human Genome Map 12q21 (2100 bp sequence)

(SEQ ID No. 64)

31A
TGTACATGCATTCATTTTGCTATCCTGCATTTGTTCTTTCTTCCTCAGAC
CTTTTCAGTCCCTAAGAAAATGGTATCATTCTGGGTAGGACCTAAGATCA
GTGATGAAATAACAGAATGCAGGATGGCAAGACTCTATATGGAGAGGGAA
ATTTTACAGAATCTAAACCTGGGGATACTAAATTAGATCAATTGAGTATA
GGCAATATCAGAGGGTAATAAAATAGTTTAAGAATACATAGATGTTTTTT
GTTTGTTGGTTAGTTTTTGCTTTTCTTGTAATTCAGGTTAAAGATGTGAC
ATTTCCTAACAGCACCAAGGAAGGAGCACCAGAGAACATGAAAAGGCACT
CAAGCTAAGTGGTCTGTTGAACTTTGCATATCTTCCTCTTTGTCAATGAA
AGAAGGCAAAACCAAACCTAAATGACAAAACAAAGAACTACTTACAAGGA
GTTTTAAAATATCTCAATCTCTGCGTTACTTATGTAACCCACAGAGTATA
TAGATGACCACTTAGAGGTATTCCTACTATTAACAAAGGTATTAGTATCT
GTGTTTTACCCATGAAAAATTAGAAACTTAGAAAAATATCTTGTTCAGTT
TCACACTGGTACAATATGAGGGTATGAAAGTTGTAACTGACTCTAAAGCT
TGAGCTATTTTCTTATAGATATAATTTAATAATGTCTATCAACTTTCTTG
AAATATTCTCATTGTTACCTAAGAATTTAAAATATTGATATGCAATTGAT
CTAAGAGAGGTTAAACATGAATTGGAAATTCCTTCTCCGATATCAGGTTT
GATTTTTCACAAATATCAATGTTTATGAAAGTATTCCTAAAATTTCAGGT
AAACCACCCAATAATATGACTGTGAAAAATTATGCTTCTTCCTCTATGAG
CTACTACATTCTTAAATTATCTGAGCATTCTATTAAAACTTAAAAAAATG
CTTAACTTGAGTCTGCATGAATCTGAATTCCCTGCATATTTAATTTTAAG
AAAATAGTTTATTTTTTTGTTAGACCAATACTTTACAAACTTCCCCAACC
AATAAGAAAGAACAAAGAGGAGAACATGAATATCCCTGGGTATTTGTGAG
TAAATCCCCAAGAGACTAACCATAAATGTGAAATTTCTTTATAATTGTAT
GTCTTCTTCTAAAATATTCATGTGGATTGTGCATTCTATTCTATCTTCTT
ATTTTAATAAAATCTGTTTTAAATTATTTACTTCCTGGAACAAATCTCCC
TGTTGTGTTGGTTTATGAACATGGTTCTATTGCCTTCAGTCTATTGTCGG
AAATAAAAACAGTCCTGCAGTTGTTGATTGAGTGTACTATGCCTTTAAGA
AGTCATGGCACTCATGCAACAGCCATGTAGTTGTTGATTGAGAGTACTGT
GTCTTAAAAAAAGAACTTTTGCTAAATAAACTGACTCTGTGAGCAGCCCT
TCATCATTTAAGTGAGAAATGTATTGAATTAAGTTACCTTGATATTGCCT
TTTGTTATATTTTTATTTCTTTGATACAAAAGAGTAACAATTTAATTCGA
AATTTGAAATCCCTGAATTGCCTATCCTCTCCAGTAAGTCACTACACACC
TGTATAGGGGAGCAGCCTTTCAGAATATTTTTCCTGAACATGAGAATATA
AAGCAGGAGGTGGTCATATTTGTTTGAGTAGCACCTCCTGATACCATTAA
TCTGAGCAGAAGAGTATGGGTCCATACTAGAACAGGATATGACTAGGAAA
ATGAAGAAGAATGAAAGCAAAGTATTCAACAGAAACATCTATGCTTTTTG
CCATTAGCTGAATGTGACATAAGAGTATAGAATGATTTGACACGATTCCA
AATCTAAATGTAACCAAGGAACTTTAAATATTATTAATGAGCATGGCAAA
GTTTAGGGTCAGGGGGAACAAATTTAAAAACTATGAGCATTCTCATGACA
TGAGTTAAAATGCAAAGACGTAAGTTAAACATAACTAATGACATTAATAA
AGTGATTGAAGCTCTATGTCACTTAAAGATAAGAAGGTATGATAGTTTAG
ACATTGTTCTAAAGGCCAATCTAAGTGAAAAAAGTTTTCAGG
Human Genome Map 17q21 (2100 bp sequence)

(SEQ ID No. 65)

32A
AGTTAGAATTCTGTGAGGTTTGTATAAAAGGAATAGAGTGGGGGCCCAAA
AACCAGTAAGATGAGAAAGTACTGTTTGCTCAGTTCTAGGATCCATGAAA
TAAATAATAAATAAATAAAAAAGAGAAAGTAGTGTTTCCTGCCACTTTAG
AGGAAGGACTCACATATCCTACCTTCCATCAGCCTTGAAGGAGATGAGTG
CCCTCTCTCCAACACCTGGTGGCCTTCCCTACCCCTTCCCCAAAGCCTCC
AAGAAGGCCCCTGGCCTAGCCTGATGCCCACTATCAGCAGGAACAGGCAC
GACAAACTTTCCCCTTCCTATCCCTCCCCACCTCTGGAAAGGGCTGGGGA
CAGCAGATGTGTCCTTGTTAGTTCCATCCATTTCAGCTTTGGCTGGGGAG
CTAATTTCACTGGAGCCAGGATAAGCATTAGGGTAAGTAACTATTTTTCC
TGTCTTGGGCAGTTTCCTCACTGACAAATGAGGGCAGAGTTCTAAGCTCT
CTTCTAATTCTAAAATTCTAATGTAAAAATTGCCAGACTAGTGGTGGCGC
AAGCCTGTAATCCCAGATACTCAGGAGGCTTAGGCAGGAGAATCGCTTGA
ACCCAGGAGGCGGAGGTTGCGGTGAGCTGAGATCGCGCCATTGTACTCCA
GCCTGGCAACAAGAGGGAACTCCATCTCAAAAAAAAAAAAAATCACCAGA
CTAATATTTACCTTGAGTGTTATGCGCATCCATGTGAAGAGACCACCAAA
CAGGCTTTGTGTGAGCAATAGTTTTTTAATCACCTGGAGTCAGCAAAAGG
AGATGGGGTGGGGCAGTTTTATAGGATTTGGGTAGGTAGTGGAAAAATTA
CAGTTAACGTGCGTTTTCTCTTGTGGGCAGGGGTGGGGGTAACAAGGTGC
TTGGTGAGGAGCTCCTGAGACTCATTGTCCAGGAGAAGGAATGTCACAAG
ATCAATTGATCAGTTAGGGTGGAGCAGGAACAAATCACAATGGTGGAATG
TCATCAGTTAAGGCAGGAACTGGCTATTTCACGTTTATGGTTCTTCAGTT
GCTTCAGGCCATCTGGATGTATATGTGCAGGTCACAGGGTTATGATGGCT
TAGCTTGGGCTCAGGGGCCTGACATTGAGGATTCTTTTTTATCTTCCTCT
GATGCTCTTCTATAAGAATGACTCTGTTTTGGAAGAAAACGCAATTAAGA
TTTTCCATCACAACAACCACTATCTCCAAATCTGTATTCATTCCTTTTAA
TTCATTATAAGTCTCATCTACCTAATGAGATAACTTTTTTGAAGACAGGA
ATTGTATGCTGTTTAACAGTGCTTTGTTTCTTCCATAGTTCAGTCATCCT
TGATATTTTGCGGGGGACTGGTTCTAGGATACTGCCCCCACACACCAGAA
TCTGTGAACGCTCAATCCCTTACATATAATGGTGCAGTATTTGAATATAA
CCAACACACATCCCCCCGCCACCACCCAATTAACTTTTTTACTTTTTTTC
CCCCCCGAGACAGAGTCTTGCCCTGTCGCCCAGGCTGGAGTGCAGTGGCA
CGATCTCGGCTCACTGCAAGCTCTGCCTCCTAGGTTCATGCCATTCTCCT
GCCTCAGCCTCCCGAGTAGCTGGGATTACAGGTGCCCGCCACCACACCGG
GCTAGTTTTTTTTTTTTTTTTTTTCTTTGTATTTTTAGTAGAGATGGGGT
TTCACCATGTTAGCGGGTGGACCTTGTGATCCGCCCACCTTGGCCTCCCA
AAGTGCTGGGATTACAGGTGTGAGCCACCATGCCCAGCCAATTTTTGTAT
TTTTAGTAGAGACGGGGTTTCACCATGTTGGCCAGGCTGGTCTCGAACAC
CTGACATCAAGTGATCCGCCCACCTTGGCCTCCCAAAGTGCTAGGATTAC
AGGCATGAGCCACCGCACCCAGCCTCAGACTAAACTATAATAAAAGAGAA
AGCAGAGAGAGTAAGAGCACCTCATATGGAATCACCTACATTTCAGAAGC
TGGAAAGAAAGAGAGTGGTCTACTTGATGATATGAAGCATGATCAATCAG
TATCAATACTAGCTTTAGGGTGAAGGCATAGCCAAATTGGAAACTGTGGA
Human Genome Map 1q32.2 (2100 bp sequence)

(SEQ ID No. 66)

33A
AAAAAAAACCTTGTCCAGGCACAGTGGCTCACATCTGTAATCCCAACACT
TTGGGAGGCCAAGGTGGGCTGATCACTTGAGGCCAGGATTTTGAAACCAG
CCTGGCCGACATTGCAAAACCCCACAAAAACTAGCCGGGTGTGGTGGCAC
ACACCTGTAATTCCAGCTACTTGGGAGGCCGAGGCACAAGAATCACCTGA
AGCTGGGAGGCAAAGGTTGCAGTGAACCAAGATCATCCCACTGCACTCCA
GCTTGTGACACAGTGAGACTGTCTCAAAAAATAATAACGAAAATAAAATA
ATCTAAAATTTAAAAAAACCCTAATTCATAGTTATGGAATTATTGAGCAT
ATTAAATAAGATAATGCATGCAAAGTACTTAACAGCATCGGACATATTTT
AAGCACTCACTGATGCTTGCTATATAGTTAAATTATATAGCTATATGTAT
GTGTATATACATGCAAAGATCAGGAGATATGCTGACATAGAATGACTATG
GCAGGGTCCTGAAAGAGACCACAAAAGAGAGAAGTTTTCACACTGGTTCT
TTCCTTTTGGGGGATGCTGACAGTTCCCCTAGAAGGCAGCAGACTTTGCC
TCTGGGAGCAAGCCACTGGCCTGGCCCAGCACTCCTGAGATGAGCAGAAA
TGGGCAGAGGAGAGCTCAAGACAGAGCACAGGCCAGACTAATGTCTTCCT
GAGAGGAGAGCAGTGGGAGGAAAAAGGGGGCAGCAAAGAGACAAGAGATT
GCCTCCTTCACCTCCACCAATGTTACCTAAGCTAAAACCCCTCTGTCTAC
CAAATCAGCCCTGGTCACAAACTAAAACCCAAACCCAACAGGAGGCTTAC
TTATCCACGCCAATCTGAATTTCTCCATGACATGGACCAGGTGGGACTGT
GGGTTTGGTGCCATGTACATGACCTGTGACTTAGTGGATGGAGTTCCTTA
GGCCACAGCAGCCTCTGGCTCAATGAAGCTTGATCTACTGAGTACCTGGA
CCACATGGGGCTCTAGCAGCAGTCCTATCTTGAGCCCAGAACAGTAACTT
TCAATAGAATATACACTGGTATGTTGAATCAGAAGTTCAAACGCCCTTCA
CCTTTATGGTGACTTTTCTCTCAAGGACCTCCACTGCTTTCTTCTACTAT
GCCTTCGTATCATTGACCATTCCATCAGTGAGGGCCACCACAGTCCCTCA
GAATTCTTTAAGACTAACTAGGGGGAGATTAGAGTACCAATCCTTCTAAA
CCTTTCAAAAGGCTTCTTTTGAACCCTTTTCAAAAGATTTCTTCACTTAG
CACCCTGGAACCAAATGGAAGTGAATATTTTTGAGAAGACGTGACATCTT
TCTCCTGGGCCTTGCCCAGCCAAAATGTTCTGTTATCTGTTGCAATTAAA
AGAGAGCAAAGAGTAAGAAGTCTCTTTCCTTAAAGTTTCTTTGGCCACTT
GAGCGGAGCTTCCCAGAGCAGTAAACTCCTTTAGGATAGGGACTGTTGGA
ATTAAATGAGCTGGGGAACCACAACCTAGAAACTGGACTTCAGCTTTGTA
AACTCCGAAACTCATTATCACTGTGATGGTTAATTTGATGTGTCAACTTG
AGTAGGCCATAGGGTGCCCAGATTAAATGTTGTTCTGGGTGTGTCCATGA
GGATGTTTCCAGATGAGAATAGCATTTGAATTGGTAGACTCGGCAAAGTA
GATTGCCCTCCTCAGTGTGGCTGGGCATTGAAAAGGCCGAAGAAAGAATT
TGCCCCTCTTTTCCTGCCTCACTATTGAGCTGGGATATCTCATTTCACCT
TCTCCTGCCCTCAAACTGGGATTTACATCATCAGCACCCCTGGCTCTCAG
GCCTTTGGACTTGGACCAAACTGCATCACTGACTTTCCTCCATCTCCAGC
TTGCAGACACAGATTATTGGACTTCTCAACCTCCATAATCATGTGAGCCA
ACTACTCATAATAAATAAATAAATAGGCTGGGTGTGGTGGCTCATGCCTG
TAATCCCAGCACTTTGGGAGGCCCAGGTGGGCAGATCACGAGGTCAGGAG
TTTGAGACCAGCCTGGCCAATATGGTGAACCCCCATCTCTACTAAAAA
Human Genome Map 5q15 (2040 bp sequence)

(SEQ ID No. 67)

34A
TACAGGATGAAGGTAACAATAAGAGAAAGTGATCATAATAGTCAATATTT
AATACATATTTAATATATATATTTTTAGATTTAACAACTGGTTAAATCTA
TTAACCATATATCATCTAACCATTTCTATACCTTCCTATCACTCTTCTTT
TCCTATTCTCTCTTAATTCCAATTTTCCTACAACACACACACTCATATAC
ACATACGCACACATGCACCCACTATCCATAAGACCATCACGTCTGGGGAT
TTTGCACATACAGAGCCTAATAAAATTCAGCAACAAAGATCATTCAAATT
CATAACTCAAAAATTATCAGGTACAGAAAATACATGAACTGAGGTGAAGA
AAAGCCAAAATACTGGGAGAAGTAGAATATCTTTCACAAAAGACCTTCTA
GGAAGATCATATGGTACTGTTCTGCAAATCTTTCCTTTTACAACATTGAA
TGTTTTAATGTGAGCTTTGCAGATTCAGTTTCAAATCTTATCACTAATCC
TGCCCTTAAGCAAAGCTGTAAAGAAGGTGAAATTAATTTTATACATTTCC
TATTCTGCATTCTGTCATCCTCATCTTCCTTTGAGGGTCTAACAACTTCC
TACCACTTTCTGCTTGTGCCCATTACAACCCAGATTTTCATCTTTTGTAC
CTGGAACAGGCCTGGCCTTCCACAATGCTCTATGTGCTATGAAAGTCAGT
TTCTGCTATTATCATTGTGTTCTATTATTTCATGTATTTTCTAATAGCCT
TAGATTACTTTGAATAGGCCTGGAATCTCTAACACAATTAAATACTATAG
CAGGCATATAGTATTACCAAGGAAAGTAAAGCAAGATATAGAACAAATTG
AATGATGAAAACTGAGACAAACTGGAGACAGGGACCATGTCCAAAATGGA
CATCCATTATTCAACTGTAGCCTCATGTTATCATGTGGGAATGAAGGCCT
AGTGTTGTAAGATTTTAGATTTTTCAAGAGGAGCCAGAAATTTGTACCTT
CATACAAAAATTTCAATATTTGTAAAACACCATAAAAGTGAAAAAAATAC
CTACAAATCAGAATTTAGACTATGTTGACATTAACTTGCTACCTCTGTGA
TAAGCGATTTTAGAATAAGATTTTATTCTTGCTTAATTCTTCTCTTCAGA
GATACCTAGTGTAGTAGGAGAATCTAACACTTAAAAACAGGTCAGGTATG
GTGTCTCACACCTATAATCCCAGCACTTTTGGAGGACAAGGTGGGTGGAT
TGCTTGAGCCTAGGAGTTTGAGAGCAGCCTGGGCAACAGAATGAGACCCT
GTCTCTACAAAAAATACAGAAATTAGTCAGCTGTAGTGGCCTACGCCTGT
ACTCCCAGCCATTTAGGATGCTGAGGTGGGAGAACACTTGAGCCCAGGAG
GTCAAGTCTGCAGTGAGCCATGATCATGCTACTGCACTCCAGGCTGGGCA
AGAGAGCAAGATCCTGTCTCATATAATAATAATAATAGTAATAATAATAA
CAATTTTGTGATGGGTGATAAATATCATAGGGGCAAAATGTCATGGGAGC
ACAGAGATGGGAGAGGGGACTTTCATAAGCCTAGAATGTTCTCAAGGAGT
ATGTCTCAGAGAATATGAATCTCAAATGAAAGTAGGCCTTTACCAGAGAG
AGAGAAAATAAAGAGTATTCCAGAGAGTTGTGTGTGGGAAGATGCAGACA
TAAGAAACAGCAGCATTTACTTGGGGAAAAAATAGTTTAGGTTCTGTTCC
CAGATAAGTGGAATTATATCAGATACAGTTTTTAAGGAGAGTCTATTTGG
GGCAGGAGGGCCTCTTGAGTTCTTATTAATAGTTTTAAAATGTGAACACA
CCTTACTGCACATTAAGCACATGTACCCCAGAACTTAAAGTATAATAAAA
AAAAATTTTAAAAAAAGAAAAAAAATGTGAACACACCTCTATTTCTCTCT
CCAGGTAATTTTAACATGACCTTGCTACTCCCTTGGATGAAAGGATCATC
ACGAAGTTTTACAACAAACTTTATGGTTATGGAAGTTCT
Human Genome Map 8p11.2 (2100 bp sequence)

(SEQ ID No. 68)

35A
TCTAAGGCTTCTGGACCTGAACTGAGCCATGCTACCAGTATTTCAGGATG
TTCAGCTTGCAGATAGCCTGTCGCGGAACTTCTCAGCCTCTAGAATCACA
TGAGTCAATTCCCCTAATAAATCTCCTTTTATCTATCTGAACATCTCTCT
TCATCTCTCCATCCATCCACTCATGTGTCCATCCATCCATCCATCTATTG
CTATCTATCTATCCATCCATGCATCCATCCATTCAACCATCCATCCACCC
ATCCATCCATCCCTGTGCCATCTATATCTATCTATCTATATATCTATCTA
TCCATGCATCCATCCATCCATCTATCCATCTATCCATCCATCACTATCTA
TCCATGCATCCACCCATCCACCCATCCATCCATCCATCCATCCATCCATC
ACTATCTATCCATCCATGCATGCATGCATCCATCCATCCATCCATCCATC
CATCCATCCATCCATTTATCGCTATCTATCTATCCATCCATGTATCCATC
CATCCATCTGTTCATCTATCACTGTCTATATATCTATGTATCTATCTATC
CATCCATCCATGCATCCATCCATGCATCCATGCATCCATCTATCACTATC
CATCCATCCATCCATCCATCCATTCATCCATCTATCTGTCTTCTACCTAC
CTACCTATCTAACTCTCTGGAGAACTCTGACTAATAAACTAGCTTTATAA
ACATGTTATTCTCTCTCTGCAATGTCTATTGCTTTATCTTCAGGAACATT
CCACACATCCTGTAAGACTTCAGTTAAATTATCTCTCTGTTTCTTCTCCA
ATCATCCTCTGCCTTCCCTAGTCTCCTAACGTACTTTGTACATCTGTCAC
AAACCCCTCATCATATTTACTGTAATTTTTTTCCTACAGATTTGGATAGG
AATTGAGCCATTTTTTTAATTTCACTTTTATGGTTGTTACAAATAAAAGA
GCAAGCAGGCCCCTCACTGTAATTCACCTGTATTTGCATTTAACTTATTA
ACCAAGGCATACTATTTCAATAATCTAATATAGTATTTCCTATTTAATAA
CCAAACATACAGAACAGTTCCAAGCACATGTAACCATGTGATACATTTTC
CTCTTTGAATAATAATATATTTCTTATAATTAATATGTGATAAAATTGCA
ATATTTTTAATCTCCTACATCCTTCTCTTTTAATCAGGTTTCCTTATCAA
CTGGTTCCTATCTCACGGGGTTGTTGCAGAGATGAGGAAAAAAAGTATTC
TATTGGTTCATGCATCTCAAAATAGGCAGATTCTTTTCTCTGCTTCTTCC
TTCATTGGCTCAGGTGTGGAGTGCTTCTCCCAATTATATGTGCCAGCCTT
GGTATGTTCTCATTGCTGTACCACACTGCCTGAGACATCCAAGACCACAT
CTTCCTTTGGGGGCACATTGGACCTTTGTCATTGGCACTGGCAGGGAAGC
TTTTATTTCACCAGGTCTAAGGCAATTCTTCCAAAAAAATCCCAAATAGT
GAAAGAATTGATTTATTCTTCTAATATTTAAGCAAATGTAAAAAAAAAGT
TACATTAGTTATGTTTTTTTCAGATTTTGGATCAGTGAGACTTCATTAAA
ACACTTTGAGGTTATAAAGCAAGTAATTTTTGTTTCCAGAAAAGTTAGTT
TCCTTTGGCTGAAGGGACATCTCTATGCAGGCCAGATCAAGACAAAAATA
ACTTTTAAGAAGGGAAATGAGGGAATGGAGTTTGGAAAACATAAATCCCA
CAGCAAAGTACGTCACCAACAATAAGAGTCATCTCTTTCACAGAGGCCTT
TCCTAGAAAAGCCCTGACAGACTAGGAGTCCAATCTTCGGCTCCCATAGC
ACCCATGCCTGCTTCCACTCTGGAGCTTACTACTTTGCGTTGAAATTAAT
TTTTACATGTCTATGGCTTCTATTACAAATAGCTTATTGAAAAGAGAACC
ATGTACATTACAATACTTTTTTAGAGTTGCTGAACTGAACAAATCAGTAC
CTACGGGGTTAGTATGCTGGCTTCTATTCCAGCAGGGTTTTGAGCCATGA
GATTTTGAATGCTCCCGACATTGTTAGTTCAGGATGATTAAAAATAT
Human Genome Map 3p12 (2040 bp sequence)

(SEQ ID No. 69)

36A
TGAGAAAATGCAAGAAAGGAAGCCAGAGACGTTGTGGACTGCAGGCTCCT
TCCCCATCATGTCACTCAAACACAATGTTTCATTTGTAAAACATATTTTA
AAAGATTATGAATGCTATTTAAAAGAACACTAAAGGCCGGGCACAGTGAC
TCACTCTTGTAATTCCAGCACTTTGGGAGGCTGAGGTGGGCAGATTACTT
GAGTTTAGGAGTTTGAGACCAGCCTGGCCAACATGGCAAGACCCTGTGTC
TACTAAAAATACAAAAAACAAACAAACAAACAAACCCAACATGGTGATGT
GTGCCTGTGGTCCCCACTACTTGAGAGGCTGAAGTGGGAAGATCACTTGA
GCCTAGTAGGTGGAGGTTGCAGTGAGCCAAGATCACACCACTGCACTCCA
GCCTGGGTGACAGAGCAAGAATACATATATATATATATATATATATATAT
ATATATATATATATATATATGTATATATATATATATATATATATATATGT
ATATATATATATATACAGCAATCAACCAAACAACAGCAGCAAAACATTAA
AGTGACATGCTGACTCCTTGGAAAGGTGGTAAGCTCTTTAGGCTTCCTAA
TCAGAGAAGGCAGATAAAGAGTAATTTAACATCTTTCTAATCTACCCCAA
TGGAATTTGCAGTGATTTTCTTTCCATTTTTTCTCATTGTTTTTCAACCT
GATCACTAATAGGTAGCTGAAATGGAGTCACTTAATGGTTTTTGCTTTTT
AAGAATTCCAAACTCACAGTTTGAAAATTATAGTTCTTGCATTTGAAGTT
TTTCTTCATCGTGCTTGGCCTGCCTGGTGGCTTTTTGTTTTGTTTTGTTT
TGGTTTGGTTTGGTTTGTGGTCTCTTTCATCTACCCAAAGCCAGTTGAAA
TAAATCAAAAGTTGTCTCACTCAGGAATTGTCTAAAGTAAATAAATGAAA
AAAAAAAAAAAAACAAGACTAATAATTAGGCAACTCATTGAGTAGGCTGT
TGAACCAGCTAAAGTGGGAAAGAAATTATTCAAGTTCTAAACCTTTCTAC
TTGCAAATTAGCCAAAATCAATTGCATTTTAAGCACTGCATCACCTTGAT
TGATTTTTAAAATGGATAGCACTTTGCTGTTCACATTTATGGTGAGCCGT
GAAGGACTTGGCAATGGGCATCTTTTCTACCGTGTTCTGCATTAAACTCT
TTAAATAGCTTCTGCTTCTTAATGTTGAATGAACTTTACTGCAGACTGAG
TCTGAGGGTTTTTTTTTCAAGGTTGAAATACATTCTTCAGACTTTACTTT
TTGCATGTAGCATTCTTCTTACTTAAAATACCTAAGAGTTTCTAACTAAT
TTCTTTCAGCCACAGGAAATATTTCTGTAATTTTAGGGTTAAAATGGAGA
ACTATAGGATGAAAATATAATACAAGAAAATGAATTAAACCCTAAAATTT
ATTATTATATGTGGGTAAAAGTAGGGGGGAAAATCACTGGTTTTGAAATT
AAAAGATGAAAATTGTGAACACTTGCATGGAGACCCTATTTGTAAACTTG
GACAAGCTTCAGTGCCCTCTCTCTGCATCTTCTGTCATTATTATTTTTCC
TCAAGGCATCGTTTTGAGGATTAAATTAGAAAATGTGAACTAGCTTGAGA
GTACAGTCAGCACTTGAACAGCATGGGTTGGAACTGCAAGAGTCCACTTA
TACACAAACTGTTTTCAAACAAGCTCTGATCTAAAATACAATATTTGTAG
GATCTAAAACACGTGTATACAGAGGGCCAACTTTTCACATATGAAGGTTC
TGCAGGGCCAGCTGCAGGACTTGAATGTGCCTGGATTTGGGTATAAACAG
GTAGTCCTGGAACCGATACACCAAGTATACTGAGGGACAATTGTAATAGG
CACAAAAATGTCGATTGATAGATTTTGTTCTTTTTCCTGAAATGCAAACC
AGGACACTTACAAAACTAAATGAATAATTACTTATACATTTAGTGTCTCT
GTGCTTCTCTCTTTTACCTTTTCCTACTTCTTCCGTAT
Human Genome Map 14q13 (2100 bp sequence)

(SEQ ID No. 70)

37A
AAGTACAATTGGCCAGGCGTGGTGGCTCATGCCTTTAATCCCAGTACTTT
GGGAGGCGAAGGTGGGCGGATCACTTGAGGTCAGGAGTTCAAGACCAGCC
TGGTCAACATGGTGAAACCCCGTCTCTACTAAACATACAAAAATTGGCTA
GGCGTGGTGGTGGCACCTGTAATCCAAACTACTCGGGAGGCTGAGGCAGG
AGAATGGCTTAAACTCAGGAGGCGGAAGTTGCAGTGAGCTGAGATCGCGC
CGCTGCATTCTAGCCTGGGCAACAGAGCAAGACTCTGTCTCAACAAAAAA
AAAAAAAAAAAAAAAAGTACAATCAGTGTTCTGTTGTGTTTTGTTGTTGG
TTTTTTTTTTTTTTTTTTCTTTTTTTAGACAGGATCTTGCTCTGTTGCCC
AGGCTACTATGCAGTGGCACAAGAACAGCTTACTGCAGCCTTGACCTCCT
GGGCTCAAGTGATCCTCCCACCTCAGCATCCATAATAGCTGGGACTACAA
GTGCACACCACCACACCCAGCCAATTTTTTAATTTTTTTGTAGAGACAGG
GTCTTACTATGTTGCCCAGGCTGGTCTCGAACTCCTAGGCTCAAGTGATC
CTCCCACCTCGGCTTCCCAAAGTACTGAGATTACATGCATGAGCCACCAT
GCCCAGCTCTATTGTGTTTCTTGTTTTTTAGCTTGACAGGAGGGTGTCAG
GGATTTCCTGGCTTACAGAAATGATATGAATTTGCAGAAGAAACTAAAAC
TGTTAACAAACATTTGAAAAAAGTTAGCCCCATTATTTATTTTTTAAATG
CAAATTAAAACAACATACCTCAGATTTAATGTAACTTCTATCAAAATCTC
TGCTGGCTTCTTTGTAGAAATTGACAAACTGATTCTAAAATTCACATGGA
AATTCAAGGGACCAAGAATAGCCAAAACAACTTAGAAAAGGAACAAAATT
GGAGGGCCCACACTTTCTGACTTCAAAACTTGCTAGAAAGCTACACTAAT
TAAGACTGTGTGGTACTGGCATAAAGACAGACAAACAGATCAATGGAATA
AAATTGAGAGTCCAGAAATAAACCTTCACATTTATGGTGAATTCATTTTT
AAGAAGGGTACCAAGATAATTCAAGTAGGAGAAAAATAGTCTTTTCAACA
AATAGTGCCAGGACAACTAGATATCCATATACCAAAAAGTGAAGTTGGAC
TCATACCAAGTACAAAAATCAACTCAAAGAGAAAAAAAACCTAAATGTAA
AACTATAAAACTCCTAGAAAAAAAGATGTAAATCATTGTTACTTTGGATT
AAACAATGGTTTCTTTTCTTTTTTTTTTTTTTTTTGAGACAGAGTCTTGC
TCTGTCACCCAGGCCGGAGTGCAGTGACACAATCTTGGTTCACTTCAACC
TTTGCCTCCCAGGTTCAAGTGATTCTCCTGCCTTAGCCTCCCAAATAGCT
GGGATTACAGGTGCCTGCCACCACGCCAGGTTAATTTTTGTATTTTCAGT
AGAGATGGGGTTTCACCATGTTGGCAAGGCTGGTCTAGAACTCCTGACCT
CAGGTGATCTGCCCACCTTGGCCTCCCAAAGTGCTGGGATTACAGGCGTG
AGCCACTGCGCCCACCCTAGACAATGGTTTCTTAAGGTACAAAACCAAAA
AGACAAGTTATGAAAGAAAAAAAATAGATTGGACATCATCACAATTAAAA
ACTTTTGTGCTTCAAAAGTCATCTTCAAGAAAGTGAAGCCAAAAACAGAA
TGGGGGGAAAATTTTGCAAATATATATCTGAGAAGGACCTAATATCCAAA
ATGCATAAAGAACTCTTACAATTCAATAATAAAAGAAAATCAATCCAATT
TTAAAATAGGCAAAGGATCTGAATGGACATTTCTCCATAGACTATACACA
AATGGCTAATAAGCATATTAAAAGATGCTCAACATCATTAGCCATCAGAG
AAACACGAGTCAAAAATCACTTACGATACCACTTCACACCTATTAGTACG
ACTATAATAAAAAAGACAGTTAACAACAAGTGCTAGCAAGGATATGGAGA
AATTAGATCCTTTATATACTGCTGGTGAGAATGTAGAATGGTACAGCCCT
Human Genome Map 4p15.31 (2100 bp sequence)

(SEQ ID No. 71)

38A
TAGAAATATTGCAATGGAAACTTCAGAAGTAAAAATGATTATAACGGGCT
ATTATAAACAATTATGTGCAATATTTAATCAGGAAGAAATAGAAAGCTTG
AATGGACCAATAAAAATTAAGAGATTGAAATATGAATTCACTTTGCAACA
AAGAAAAGCCCAGGACGAGATGGCTTCATGAATGAATTCTACTAAACATT
CAAAGAAGTATTACCAATATTTAAATTCTCCCAACAAATAGAGATAGAAG
AAATACCTGCAAACACATTTTACAAGGCAAGCATCACCTTGATCCCTAAG
CCAATGACATCACAAAAAAGAAAACTATAGGCCAATATCTCTGATGAACA
TTGATGGAATTCTCAATAAAATATTAGCAAACAAAATTCAACATCACATC
AAAAAGATTATACATCATGACCAATAGGATTTATCCCTAGCATGCAAGGC
TGGTTTAATACACGAATGAAACAATGTGACACATCACATTAACAGGATGA
AAGATAAAAAACACAGAATTTTCTCAATCAACACAGAAAAAGCATTTGAC
AAAGTTCAGCATCCTTTCCTGATAAAAACTCTTAACAGTTTATGTATAGA
AAGAAAATTTCTCAACATAATATAATAAAGGTGATTTATGAAAAATCCAC
AGCTAACATAATAATCAGTGGGAAACAGTTGAAAGCTTTTTCACTAAGAT
CCAGTGCAAGCACAAATGCCCACTTTTGCTACTTCTATTCCACATAATAT
TGGAAGTACTAGCAATAGCAATCAGACCAGAGAAAGAAATAAAAAGCATT
TAAGTCAGAAAGAAGAAAAAGTAAAATTATCTCTATTTGCAGATGATATA
ATCCCTTATGTAGAAAACCCTAAAGATTCCACAAAAAACTGACAGAATGA
TTAATTCAGTAAACTTGCAGGATACAAAATCAACATACAAAAATCAGTAG
CATTTTTATACACTAATAACAACATATCTGAAAGACGCTTTAAATCCCAT
TTATGAAAGCATAAAAATAGTTAGAAATAAATTTAACCATAAAGGTGAAA
TATTTGTATACCGATAACTATAAACCTTTGATAAAAAAAGTTGAAGAAGA
CACATATAAATAGAATAATATTCTGTGTTCATGAATCAAAAAATTTAACA
ATGTTAAAATGTCTGTATTAACCAAAGCAATATACAAATTCAATGCAATT
TCTATCAAAATTTCAAGGATATGCATCACAGAAATAGAAAAAAAATTCTT
GAAATTCATATGGAACCACAGACACATAAAAACAGAATAGGCAAAGGAAC
AATGAGAAAGCAAAACAAAGCTTGAGGCATCACACTTCCTAAGTTAAAAT
TATATTGCAAAGCTACAGTAATCAAAAACAGTATACAAATGGCATGAAAA
CGAAAATGTGGACCAACGGAACAGAATATAGAGAGCCAGAAACTTAACTA
ATTTTCAACAAGGGTACCAACAGGACACCCTGAAGTAAAGATAGTTTCTT
CAATAATGATTCTGGGAATTGGATTGCACATGCAGAAGAATGAAATTGGA
CCCTAATCTTGCACCATATACAAAAATGGACTCAAAATAGATAGGAGACC
TAAATGTAAGATGTGAAACCATAAAACTCCTAGAGAAGAACATAGGGGGA
AAAATTCCTTGATATTGGCCTTGGAGATGATTTTTGGATATCACACCAAA
AGCTTAGGCTACAGAATCGAAAATAAATAAATGGAACTACATCAAACTAC
AAAGTGTCTGCACAGTAAAGGAATCAATCAACCAAATAAAAAGGCAACAT
ACAGACTGGGAAATATATTTTCACACAGCATATCTCCTAAGAGGCTAATA
TTCAACATTTGTAAAGAACACTTACAAATGAGTAACAGAAACAACAAACA
GCTTGATTAAAAACAGGCAAGGGACCTGAACATACTTTTCTCCAAAGGAG
AAATAATGGCTAACAGGATATGAAAAGGTATACAACATTGCTAATCATTA
GGGAAACACAAATGAAAACCACTATGAGATATCACCCTTCACCCATTAGG
ATGGCTATTATAAAAAAAAAAAAAGACAAGA
Human Genome Map Xp11.3 (2100 bp sequence)

(SEQ ID No. 72)

39A
AGTAAGTGGTGGAGCCAAGAATTGAACCACAATGTTCAGGATTCATAGAA
TGATTAGAATATAGTGAAAACAAAGCAAGAGATAAATTAGAGAGCTGGCA
GGCGGGGCCCACTCATGAGAGATGCATGTGTACGCCATGTAGGATACCCA
GGTTTTCTTCTTTTGGTGAACGGAATCTCTTGAAGGGTGTTGAGCAAGAC
AGCAATATGAGCAGAGAGTTAGAAGCCCCACCACTCAGGAAGCAATGCAG
AGGTAGGCACGGGAGTGCATCTGAGATGAAGAGAATGGGCTGAGGTTGCA
GCTCAGAGGCATAAACTAGAATTGAATGTGCTGGGCCTCGGATGGACTCT
AAGCCTCTTGTTGAGTGTTAGGGAGACAGGGCCATCATGGAAGCCAGCTT
TGGACAGCAGGAGGGACACCTTTTCTACCGAGATGAAAAAGGAATGGCAA
GTTGTGGATGTGGGGGCATGGGTGTGGTTTACATCTCAGGATCTCTATAT
GCTCTATTAACCTGQAGATGAGGTTTTCTGTTATAAATTGGAGGAAAATG
GTGAAGTTAGGAGCTTAAGAGTGAGAAAGTCTGAAATATCCATTGTGGAG
AGTGGAAACAGGTACAACTAGGCCTTGATTCCCTCCTTAACTGCTTCTTC
ATCCTCAGCTACTCACTGATCCATCAGTTGTCCAGTTTGTCATCTGTGAC
TTTGATTTCTATTGGCCCTTTCCTTTTGGACCTTATTGGTCCTTTCCTTT
CAGCATATAGAATGTTCATGTCTCTCTCAACTTAAAACAACAGCAAAACC
CAGCCCATGACCTTCTGCCCGGTAAAGTCATCCTGGTAGTGTCCACCAGC
CTGGCAGAACCTGTCTTCCACAGCAGCCTGGTGACAACAAGAAGCCACAG
CAGCAGTGTTTGGCAGTGGTCTGGCCCTCTCCAGACAAAATAAGCATCCC
TGGGGTTCCTGGACAATGAAGGGCAAAGGAAAAGAGAACAGGTCTCAAGA
ATATACTGACATCTCCAAGAGGAATTCAAGATAATATGGCAAATGGGAAT
TAAATGAGAAAGTAAAAGAGAGGTGACCATATGCAATCTGGAGCAGGTGG
AATGTGTCATGATGGTTCAAGGGCAATCAAGAGTTCACATTTATGGTGAG
GATTTCTCTTGATTTTTTTTCCTCCTGTCATCTTATTTTGTGTTTTCAGT
TTTTCATGTATTGTTACCATTACCTTATATTGATTGCTTCTTGTCTTTTG
CAATACATCCTAAATATAGCAGGTTCTTGAATAACATCACCTCGTTCAAC
ATCATTTTATGATAATGTTGATGGGGAAAAAAAAATGGCTCCAGGCTGGA
GCCACTGTCTGCATGAAGTTTGTACTTTCTCTCCATGTCTGCGTGGGTTT
TCTCTGGTTTCTTCTCACATCCCAAAGCTGTGCACATGAGGTGAGCTGGC
ATGTCTATATGGTCCCAGTGTGACTGAGTGTGGATGTGTGAGTGCACCCT
GCGATGGGATGGTGTCCTGTCCAGGGCTGGTTCCCACCTTGTACCCTGAG
CTGCTGGGACAGGCTCCAGCCACCCATGACCTTGAACTGGAATAAGCACA
TTGGAAAATGAATGAACAAATGAATACAAATTAGGATAAAATAAAAACTC
ATCAAGTCTATGACAATAAAGGACATGGGACAAAAGCGCTCAGCAAGCCT
GCTCTACTTGTGATTTTTTGGTTTTGAACTGCAAGGTGGGAAAAGATGCT
CCTTACAATGTTCGCTCTGCAAACATTTATTCCCTGATTTAACCCATCAC
TACCATGGCCACTGCCACTCACTGATTCACCAATTGGGTAAATCATTGTC
TTGTTTTTATTAATTTTTTTTTTTGAGACAGAGTTTTACTCTTGTTGCCC
AGGCTGGAGTGCAATGGTGTGATCTCAGCTCACTGTAACCTCCGCCTCCC
GGGTTTAAGCGATTCTCCTGCCTCAGTCTCCCGAGTAGCTGGAATTACAG
GCGCCCGCCACCACACCCAGCTAATTTTTTGTATTTTTCGTAGAGATGGG
GTTTCACCATGTTGGCCAGGCTGGTCTCGAACTCCTGACCTTAGG
Human Genome Map 11p15 (2100 bp sequence)

(SEQ ID No. 73)

40A
AAAGGCTTCCTTCCCTCAACAAAAAGGATCTCACCATTCTTTATATTCCA
GGTTTACTTTCTGATTTACCCGTACAGTATCATAGCCTGGAGGCTCCTGA
GATGCATTCTTTTTTGGAAGGGGCCTGATTTTGAAGTCTGACTCAGCCAC
TGATTAGTGAGCAGGCTTTCCTAAATCTTCCTTTCCTCTCTAGTAAAGTA
GGTCAGATCGTAATCTCTTCCTCACAGGACCATTGTGCAAATTAAAGGAG
ATAACGTGAATGGTTTGGTAGCAACTCAATGAATGTATCTGAATCCTCCC
GTCAATTTACTCTGCTGTCCATTATAGTCAGTTATGTATCTGTTTTATCC
TGTCTGGTCGAGAATGGAATCTGAGGACCAAAACTCCTCTCTTACTCATC
TCTGTGCATTTTCCTCTTTGCCCCTGCCCCTTCCTCCAACCCAGTACCTA
GCATGGTTTCTTCATTTATGTGTAAATTTTATCAAAGTGGTGTCAGTCTG
TGCAAATCATCTGCTGAAGTCTGTTAAGAACGTTCAGCAATATACACTTC
AACAGTAACAGGGACAGTGTGGAAAACCCTTGTTTTCTTCCTACTCTCCA
ATTCTTCTGCCTCTTTCCACTGCTTTGGAGAGGAGGTTACAATCAGAGTC
ATTTCACCATAAGAAAATCATCAATTTTCTAATGAAAACCCCCCTCCTTA
AATATTCAGCCGTGGCACAATCCCTAAAGGAGAGAACACATTAATGCAAT
GCACTTGGCCAATTTGTGAGCCCTGACTTGTTGAGCACAGACTCTTCCCT
GACCCTGGAAGACAATCGGCAGGACCCCTGAATAATGAAGTATGAATGCT
CACCATTGTGCTATCTCTAGCTACACCAGCTTCTACCTAATCTTTTTTTT
CCTTTTCATTGCTCTCCTTTGCAACTTCCTAAGGATTTGGCCATTTTCCA
CTGTGTCCAACCTACGAAGCTGTAAATACCTGACTGAGAAACACAAAATG
TGTTTATAGGACTTCTGATGGCTTCTCATCTAAACCATAAATGTGAAATG
CATTTCAACATTTCTCAGAAAACACCCCATCCCCCAGATTCAATTGGCAG
CACAAATTTACTTCTCAGAAGACAGCACCAAAATCTCAACATTGGCATT
TTGAATCAAGCACACACACGCAGCTGCATTGTGTTAACAAAGTGAAACAT
TATTAGGTCGCATACTCTGAGTGACAATATCCTCGAATGATCATTTCTGT
GAGAAATTATTAGCTATGAGAAATTCAATTTGGTAGCATTTGGGCATTAC
AGACAGCTAGTCTGTTTATATCAGCAAGGCTTTGATGTTAAGACTGTGTA
ACTGCAGCCACAGGAAAAGCAGACTGAATACAGGGTGGATAAGGTCACAG
ATATAAAAATCAGATAGAGTTCTGTTCTATTATCTACATAGTGTGTACTT
TGGGAAGTTACTTAATATTTCTAAGCCTCAGTTTCCTCATAAAAATAAAA
ATGGCAAGCAATATGAAAACTATCTAATAGAATATTTGTGACACTAAATT
GTAATAATGTATATAAATCACTTAGCCTAGTATGTGGCATTTATTAACAC
TCAAGCAJAGTGTAATTTTTTAAAAAAACTCTTATATCCCTTACATGACA
GAAATATTAAGACCAAAAATGGTTACTGAGCCCTCAAAGGTATTATCTCA
TTCTGGCTGAGCTATCTGGACCTGGAGA4AGTCTAGAAAAGACTCTATTT
CACTCCAAGTTTCTTGACCCTATCTTTATTTTTTATCTTCTATCCACTAG
GACCTGTGATCAGGCCAGGATTAACCAGTGTTCTCTAGGATTAACGTTTT
TGGCAGCTGGGGATGATTGCCTAAGATAATTGTTTTTGTGTCTGCCTCTC
CTGCTAGAATGCAAACTCTCAAGGGCAGGACATATGTCTTTCTTTATCCT
ACCTGTTAGTGATCAACAGGAGAAGGCCACTGCTTAACTGTTAGTGTCAG
GTCAGCTCCAAGCTGGTACTTCTTAGGAACTCTTTTCTTTTCTTTTTCTT
TTTCCTTCCTTCCTTCCATTTCTTTCTCTTTCTTCCTTCCTTCCTTCTGT
Human Genome Map 2q31 (2100 bp sequence)

(SEQ ID No. 74)

41A
CACGTGAGGACACAGCATAATAGTAGCCACCTGCAAGCAAAGGAGAGAGG
CCCAGGAGAAACAAGCCCTGCCAGCACCTTACTTAATCTTAGACTTCCAG
CCTCCAGAATTGTGAGAAAATAAATTTCTGTTGTTTAAGCTATCCAGCCT
GTGATATTTTGTTATGGCAGCCCCAATAACTAGTATGTGTATAATGAAGC
CCTAGACAACAAGGGACTCTCATTTCTCCGCATATTTGTAGAACTCATCC
CAATTATATAGAGCTCCTACTCTGAGTGCTAGACACAGTGTTAAACACTT
TCCCTGTGTTATCTCGTTTAACCATTAAGCTGAATCCTCCAAAACCCTTT
GGAAATCAGACTTATCTAAGAAACTCACTATTGTAGTGAAGCTGTTTTAA
AGAAGAATTGAAGGTATTTTTCTTTATCTTATAATCTGTTACATTGTGTT
ACATTTTAAGATAATACTAATCTAAGGACTGATAACAATTTAATTTGCCA
GAATCATTAAACCAAATAACATCTTTAACAGTGGCTGCTAGACAGGGGCA
GCTGTATATTTTAATGCCATATTTGGGGGAAAAAAAACAGGGTAGCAAAC
ATATCTATAAATAAGAATTAATTGCTACAAATTACCTGGGAAGGGAAAAA
TGTCAAGTTCATATAAAGAATATTATTGACCCATGGATTTACAGCTATAT
AATAATTTGGTACCTGGTTTATTTCTTAAAGACCTAGCACGTTTCTTGTT
TTCTCCTGCTATATTACGTGTACATGGCGTTTCAATAATCAAGCAAAAAA
GATGTATGCACTATCTTAGTCTTTGTTGTCTAATTAAAACTTTTTATGCA
TAGCAATTGCTTACCATTTTGCATTATCACCAGAGCTCATTTCTCATGGA
AAAAAAATTAGCATCAGTTTAAAAGAATATTTCTTTAATCAACAGTTCTG
ATTGTCAGTAGTACCATTTTGTAGATAGTTTTTAGCTGACTAACAAATCT
TTTATTTTATTGGCTGTCTCATTTTGCTCTCTTGCATATTTCACATTTAT
GTCTATTCAGACATTCTCCTGTTTTGTTAAGTGGAAATCTGTGTGGTCT
TTGATGTAAGACATAATTTATTTGACAAGGAAATATGAGTCTGTGCCCTG
AATCCACATTTAACTGATGGATTGAGAAATTTTAAAATTGCAACAAGATA
GACTCTCCTCCAGATTGCCGTACTACTTGCATTTTGCTTATCTATTTGGG
AGTGAATTTACATATGTGTGTCTATATACGAATATATAGAGAGTCATACA
ACCATGCAGCTGTACTTGTGCAATTTTTCTACTTTGTTAATAGAAAATGC
AGTCTCATTTTGTTAGTCATTAATGGTTCCTATAGAAAATTTTTAAAGAA
TTTTTTTCTGAAATTAAATTCAAGATACTTATTATGTTTTATCTTCATAT
AGATAGCTTTATAAAGAGAGTGATGTCTTCAAGTCTGTACTGCTCGCTTC
TCAGCCTAGTAAATGGAAGTTTTGTTAGCATTTCAAGATTTATATATTTC
ATATGTTCTCCCAAGTCTATGGCCCAGTTCTCTGTAATGGAAACTTACTT
TCAGCTCATTCCCTCTGCTCAGACTACTTGTCAATTAACCTTTGCAAAAT
GATAGTTTTAAAAAATATGACTTTCATATTTCAATCATGTTCATTTTCAA
TCATCTCAAAATGTAGAAATTGAATAACACCCGGGGTTCTACAGTGCTTT
TTACATATCATTTAAGGTTTAAAACATCTCTTTGATGTTCAAATATGACT
GCCATTTATATTCAATGGATGAGATTAAGTGGTTAAAATTACTTGTACTG
GGCATGCCCCTGCTTTGTTTATAGGTATGAACAAAACACTAAGGATTTTT
CATAAATATGCACCATTTCCATTGATGTTTTTGACTGCTGTCTGTGACAC
ACTAGGTAGGCCATATTAAGTAATGGGGAAGAAATCATAGGTCCTACTGT
GATATTAAAAATTTACATTTTGATGAATTAAATAGAGTTGTTGACCATTC
TACACTGTTGATTATATGAAGGGAAAAAGCTAACAACTTCTAAGAATAA
Human Genome Map 3p12.3 (2040 bp sequence)

(SEQ ID No. 75)

42A
AAAGGTAAAAGAGAGAAGCACAGAGACACTAAATGTATAAGTAATTATTC
ATTTAGTTTTGTAAGTGTCCTGGTTTGCATTTCAGGAAAGAACAAAATCT
ATCAATCGACATTTTTGTGCCTATTACAATTGTCCCTCAGTATACTTGGT
GTATCGGTTCCAGGACTACCTGTTTATACCCAAATCCAGGCACATTCAAG
TCCTGCAGCTGGCCCTGCAGAACCTTCATATGTGAAAAGTTGGCCCTCTG
TATACACGTGTTTTAGATCCTACAAATATTGTATTTTAGATCAGAGCTTG
TTTGAAAACAGTTTGTGTATAAGTGGACTCTTGCAGTTCCAACCCATGCT
GTTCAAGTGCTGACTGTACTCTCAAGCTAGTTCACATTTTCTAATTTAAT
CCTCAAAACGATGCCTTGAGGAAAAATAATAATGACAGAAGATGCAGAGA
GAGGGCACTGAAGCTTGTCCAAGTTTACAAATAGGGTCTCCATGCAAGTG
TTCACAATTTTCATCTTTTAATTTCAAAACCAGTGATTTTCCCCCCTACT
TTTACCCACATATAATAATAAATTTTAGGGTTTAATTCATTTTCTTGTATT
ATATTTTCATCCTATAGTTCTCCATTTTAACCCTAAAATTACAGAAATATT
TCCTGTGGCTGAAAGAAATTAGTTAGAAACTCTTAGGTATTTTAAGTAAG
AAGAATGCTACATGCAAAAAGTAAAGTCTGAAGAATGTATTTCAACCTTG
AAAAAAAAACCCTCAGACTCAGTCTGCAGTAAAGTTCATTCAACATTAAG
AAGCAGAAGCTATTTAAAGAGTTTAATGCAGAACACGGTAGAAAAGATGC
CCATTGCCAAGTCCTTCACGGCTCACCATAAATGTGAACAGCAAAGTGCT
ATCCATTTTAAAAATCAATCAAGGTGATGCAGTGCTTAAAATGCAATTGA
TTTTGGCTAATTTGCAAGTAGAAAGGTTTAGAACTTGAATAATTTCTTTC
CCACTTTAGCTGGTTCAACAGCCTACTCAATGAGTTGCCTAATTATTAGT
CTTGTTTTTTTTTTTTTTTTCATTTATTTACTTTAGACAATTCCTGAGTG
AGACAACTTTTGATTTATTTCAACTGGCTTTGGGTAGATGAAAGAGACCA
CAAACCAAACCAAACCAAAACAAAACAAAACAAAAAGCCACCAGGCAGGC
CAAGCACGATGAAGAAAAACTTCAAATGCAAGAACTATAATTTTCAAACT
GTGAGTTTGGAATTCTTAAAAAGCAAAAACCATTAAGTGACTCCATTTCA
GCTACCTATTAGTGATCAGGTTGAAAAACAATGAGAAAAAATGGAAAGAA
AATCACTGCAAATTCCATTGGGGTAGATTAGAAAGATGTTAAATTACTCT
TTTTATCTGCCTTCTCTGATTAGGAAGCCTAAAGAGCTTACCACCTTTCC
AAGGAGTCAGCATGTCACTTTAATGTTTTGCTGCTGTTGTTTGGTTGATT
GCTGTATATATATATATATACATATATATATATATATATATATATATATA
TATATACATATATATATATATATATATATATATATATATATATATATATA
TATATATATATGTATTCTTGCTCTGTCACCCAGGCTGGAGTGCAGTGGTG
TGATCTTGGCTCACTGCAACCTCCACCTACTAGGCTCAAGTGATCTTCCC
ACTTCAGCCTCTCAAGTAGTGGGGACCACAGGCACACATCACCATGTTGG
GTTTGTTTGTTTGTTTGTTTTTTGTATTTTTAGTAGACACAGGGTCTTGC
CATGTTGGCCAGGCTGGTCTCAAACTCCTAAACTCAAGTAATCTGCCCAC
CTCAGCCTCCCAAAGTGCTGGAATTACAAGAGTGAGTCACTGTGCCCGGC
CTTTAGTGTTCTTTTAAATAGCATTCATAATCTTTTAAAATATGTTTTAC
AAATGAAACATTGTGTTTGAGTGACATGATGGGGAAGGAGCCTGCAGTCC
ACAACGTCTCTGGCTTCCTTTCTTGCATTTTCTCAG
Human Genome Map 4q13.3 (2040 bp sequence)

(SEQ ID No. 76)

43A
CTCAGTTTTAAATGTTTCCATCAATCAGTAATTCAGCTCCAGAGTTGCCA
TAGAAGGTTATGGGAAAAAAATCCTTCTGCTTTTCCAATATCAAAGAGAG
AGATGTTCTGGAAGGTTTTATTTTTGCCACCCTGTTTCTAATGCATTTCG
CCTTAAGAATAATATTACTCATCTCCAGCAATCAGGCTCAAGGAGGAAAT
TTGATACCATTCTGTGGGTCATCCCCAGATCTCTGCAGGCTTCTGGCAGA
TGTATGATGTAGTGGGCACCACTAACTTTGTCTGCTAGGGTATTAGTAGG
ACGTGGTCTACTGAATACCGGACAGACATTTGGAAATTAAACTAATCAAA
ATAGGCTAATTTCACGTGCTAAGCAATGCAATTTCCCTGAATTTGTAGTT
CCATGATCTACTTTTTCTTCTACATTTCTCTTACTCCCTCTCTTCTCCGT
TTACAACACAAAATTCAACAAACTGCTAACTCTAGCTATTAATATCCCAT
AGTATTTCTCTAAGCAGCTTCATAGTCACAGTTTCAGTTAAGGCTCTAGG
GCTTTCATTCCTGAACTACGTGTACTACAGATCCCAGTTAAAATCTCTAT
CCTTCCCACAACAGTGGTTGACCACCTTCCTCAACATTGGATTTGGGGTG
ATAATGACAACATTCTGAACAAAGCTCATTTTTTCCCCTAACCCCCAGCT
AAATAGAAGAAATGTTAATGTTACTCCTCTTTAGATTTTTTTAATTAAGA
ATATTTTTAAAGGATTTTTTTATGATTTAATGGGACACAATGAAAAGATA
TTTTGACAAAGGTAAACATCTGAAACTGAGGCAAAGAATTTTAGATGTTG
CCTGTGTAACCATTATTCCATGATCAAAAGCCCAATGTTTAACATATCCT
GATTTCATCATTAAACAAGCATAAGAAAAAAAAAAAAGAAAACCATAAAA
GTGAAATAGATTACATCTTGTAATAATACAGCTATGAAATTCTGACCAGA
ATGAAAATATGAGTATGAAGAGAGTAATCATTTGCTTATTAATTCAAGGA
ACAATTTGCCATTTTTCAAGTATTATGAAAATAAGAGACTGTTGGACTCT
TTTTAAACACGCAGGTTTTTCAAATGTATGTACAGTAATATTATAGCTCT
GGTGAAAATTTTGATGAAAACAAAATTTTCTGTCTTCTTTTACTTACCTT
GCCCTTTTCAAAAAATAGTGCTTAATGTTAACCAATGGACAGTCTAACTA
CCTGAAGCTTTTCATTCAGCTTTATTTTCTCAGCAACTATGGTTGAAACT
GACAAGTTAGGTGAAAGGTTGTGTAAGTATCCAGGCAGGGGCAAAAATAT
CGAGTTATCCCCAAATACTAACAAGCACATAGGTAGAATATTTCTACCAA
GTTAAAGAGAATAAAGGAACCACATTGAGCAGAGCTACCTTATTCAAGGA
CCTCATTATCTCAAGGCACCCCAATTGAATAAGTGTACCATTATTCCCTT
CGTTCTGTGCAAACAGCAGACGTAGAGCACAAAGAGAGACGATTTCAGTG
AATCACACTGTAATTATAAATGCCACATTAAAAAACGGAACAAAAGCAAC
AGCAGGACACTGTAACGTCGATGGTTAAGGAGGGCAAACAGAGAAACATT
CTCAAAGGCCACAATAAATTACATGATCCAGTTCTTTGTTACAGGCAAAT
TATTGGACAATAAGAGAGACACTGAACACAACTTATCAGTGGTAAAGTAA
CTTGCACACATTCTCCTACCACTGAAATATTCCTGTTACCTACACAACTG
CATTTGTATATACAAGCAAGAATTTTGATGCACTAAGTAATTAATATAGC
TCTCTAGTATTTTTTTTACCTCTGTGCTTATTCTATCTGGGCAAGGTGTG
GTAATAGCACCTTAAAAAATAAGATCAGATTTAGGAGTGAAGAACACTGC
ATTGGAAAAGGTAATTGCTAATTTTATTGTATTTTAATTATTTGACCATT
TGTGCACAAAATTAAATAATACTTGCTTCATCTCTATATT
Human Genome Map 4q13.3 (2160 bp sequence)

(SEQ ID No. 77)

44A
GTGTATTGCTGTGAAAAAAGTCACTTCAAATTTAGTAGCTTCAACCAACA
ATCATTTTATTTGCTACCAATTTTTTGGGTGAGAAGTTTGTGCTGGGCTC
AGTTGGACAGTTCTACTGATTTCCCTGTATATACCTCATGCAGCAAAAGT
CATCCTGGTGGTTCTATGTGATGGTTAATTTTTGTATCGACTTGGCTGGA
TGATAGTCCCCAGATATTTGGTCAAACGTCATTCTAAATATTTCTGTCTA
TGTGTTTTGGGAATGAGATAATATTTTACTGGATTATTCTTCACAATGCG
GGTGGGCCTCATCCAGTTAGTTGAAGGCCTTAATAGACAATAATTGACAT
CAGACCTAAGTAAGAAGGAATTCTGCCAGCAGACTGCTTTTGGACTCAAA
TTGCAACTCTTCCCTGAGTCTCAAGCCTGCTGGCCTACCTTGCATATTTT
GGACTTACCAAGCTTCCACGACTGTATGAAGCATTTCCTTAAAATAAATG
TACATATCTGTCTCTCTACACACATACATATATACACACTCTCACACACA
TCCTGTTGGTTCTCTTTCTCTGGAGAACCCTAACACACTCCACTCAGACA
GATTGTCTAAGGTGGCTTCACTTAAGTTTTGGATATCGACTCTTGCCGTC
ACTCTCTTGAAACAATGTTCTTAGACTCTCATGTAAAGAAACTCCATCTA
GCTGCACCCTCGGCACTGTCCCAAAAAAGTGAGGATAATACCTGCAAGGT
TTCTTAAGGATTATGCCTTGAAGTCATATTTTACTTCTGTAGAGTTCTAT
TAGATCAAACAAGTTACCTGGCCAAGCCTAGGGTCATAGTGGGAGAATTT
TGAGGGCCATATGGCAAACAGCCCACCATACAATACATTCTCAAATGGCT
TCTCAAATTTTACATTCTTGTGAATGATTCTCTCTTCTTGTGATTAATTT
TACACACATTACTTCAATATAAATTTTTATCTCATTGCATTTTCAGTTTT
TTTGTAATTTCACATTTATGGTACTCTTACAATGTGCCATGTGCTATTCT
AAGTATTTTATGTAGATTAATTTAATTCTTACAACAACCTTTTGAGGTAG
GTAATACTATTTTGTTCCCATTTTCAGATGAAAATGCCAAGGCATAGAAA
GCTTATGTAACTTGCCCAGTAACACTCAGAGACTTAATGTCACAACCAGT
ACTTAAATGTATACTATCTGACTACAGGGTATGCATGCTTAGTCATAATG
TTATTAAAATATCATTTGTGATGACTGAGGCATCATGGCAGATAGGAGGC
AGGACTAGATTGCAGTTCCAGACAGAGCAGGAGACAGAGGCTTGCACATT
GAATTTTAGCTCCAGATCGACTGCAAGAGCAAACCGGTAATCCTGAGAGG
ACCCACAGATCCTCTGCCGGAAGCAGACTGTTTCTGCAGGACCAAGGAGA
CACCACAGATACTGTGGGTGTCCCAACTGCAGAAATTGGAAAGGGAGACC
CTTCTCTTCCAAACACACACCCCACTGGAGAAGCTGTTTCTGACTTTACC
TGGAGCTGAGTCAAGTTAGAGAGCTGAGCCAAGTGAAATACAGGGGTAGG
GGAAGTAGCGGAAAGACCCTGGGAGCTCGCTGGGTCCCCCAAGCAGCCCA
TACCTGCCTGGCACCACAGGGATCCACTGGGAGGTTGGCCAGAGAAGTAG
GGGGTAAAATACCACAGGCAGAAGGAATTCTCTAGCTAAACTCTGTAACA
ATTTGAACGGGGCATGAAGCCTCCTGGCCAGTACTTCAAGGAGGGTGTGA
ATCCAGCATGCAGACCTCACAGGCAGGGGGGAAAACTAAAGCCCTTTTCT
TTGGCAGCCGGGAGGTGGAAAGCCTCAGGCAAGTTTTCAAGCAGGGCTCA
CCCTCCACCTGGAAACAGACTCCAGGTTGTTGAGGGGGACACGGTGGGAG
TGAGACTGGCCCTTCAGCTAGCATGTGAACTAGGTGAGGCCTGTGACTGC
TGGCTTTCCCCTACTTACCTGACAACCTACATGACTCAGCAGAGGCAGCC
GTACTTCTCCTAGTGTGTCCGGAATTGGTGGGTTCTTGGTCTCGCTGTCT
TCAAGGATGAAGCCGCGGACCCTCACGGTGAGTGTTACAGTTCTTAAAGA
TGGTGTGTCCG
Human Genome Map 1p14 (2100 bp sequence)

(SEQ ID No. 78)

45A
CCTCCCACAAGGTCCAACTCAATCAGAACCAAAAGGGAGATCACAGCATA
TCCATGCAATCCCTGGCTGGACAGACGGGGCACCCTAGGGCCTGGAGTTA
CGCAGCTGACTGGCAGAGGTCAGTACCCAGTTCTACCCAGTGTGGCCACC
CGATCCAACTCTGTGACTCATTTTAAATCATAAGTAGTTTGAACAAAGAC
TTAAAATTAACGGGTTTGGTCATCATTAAAGTTTATTTTTAAACGTCAAG
TAATTTGGCATTTTACTAACAACAACTTGAGGATATCCACAGTGTATGAA
ACACCAACTCTTGTTTCAATAAAAGTCTAATGAAAGTTTCTCAAATTCTG
AAACCTAATTCCAGGATTCTTATTTGGAACTGTTTTTTTGTTTTTGTTTT
TGTTTTTGTTTTATCAGAGTACATTAACTCAGGGGGAAAATGAGATTATC
TTTTGATTCAGAGAGAAACAGAACATTCCACTGATAGTTTAAAAATAACA
CAGTGACCACAGATAACTGTAGTTCAGCAATTTTCAAATTCAGATTCTGG
GTCAGGGCACAAGATTATGCGTCTTTAACAAGCACAACTGATTATAATGC
TGATGGTCTAGAGGAAACTTGATGGGAAACACTGATCTATTCAACACTTT
TTAAAAGTACACTTTAGTACTATTTCTACCTAGTCAATAAAATAAAGAAG
GAAGGAGAAAGGAGGACGAATAAGAGAGAATCCGAAAGACACACTACCCA
GACAGACTACTCAGACAGACGAGACAGTCAGGCAGACATGGTGCTACCCG
TCAAGCATGCAGGAAAGCAGCTTTCATGGAATAACATTCCTAAACTCTTG
CCTAATAAATTATGCTGAGAGCTGCTGCTAAGAATTTTTTAAACCAACTC
AAAGCAAAAAAGGAGCTATTCACCCAATCAACAGGTGAAATCAAGTCACA
GACTAGTATAGGGTTTGGCAGATTTCAGGCCCTCCAGAAATATCTGTTTA
ATTGAGAAGCAACTCCAGCTCTAGCTAGAAATCTATTTAACCATAAAAGT
GAAATCATAATGAATTTGGTCGTATCTTATTTTTCCCCTTTGTTTGTTTC
CTCGGGGCATCTATAATGGCTGAATTGGAAATGGAACCACAAGTATTATA
ACAACATTTGTTGGAAAGTTCATCCTGTATTTTAGTAGTACATAAGTTGA
CAGATATGGCTTTATGAATTGTTCTCAGAGACTTAAAAAAAAAAAACCCT
GAACTTTGTAAAAATTACATCCATTATCCAcCAAGTAACATTTGCAAGCA
AAACCCTCTACTAGAAAAAATGGGTGCGAAAATAGGAAAAGGAGAAGAAC
AGGAGGAAGAAAAGGAGAACAGGACGTACAATTAATTGAGGGGAAAAAAA
TCATGAGTAAAGAAGTCAGAAATAAATGTAGCTAAAAATACAAACTGCTA
CTTTATGGTCCAGATATTGTAATATATCATTTTTAACATAAAAGAAAAAC
AAATCCTCAACAGACTTCCTATAAACGAAATTATCAGAGTTCCCGAGTAC
ACCGGGGGTCGAGGGAAGAATCTCCATGTGCTCCGAGTATCGATAGCCAG
TCCAGCTTCATTCACTCATTCATTTCTTTTCTTTCATTTCAGGAGAACAT
TTAGCAGTGTTTTGTTTTATTTATTTTATTCAAAGGGAAATCCTCATGTG
ACACTAGCGGTGAAAATAACTTGTATTTGTAAGTTAATGTCTGCTGTACA
TCTGAGTACACAATTGTCTTTCACAGAAGATGGAGCAAAGTATTACGGAA
AGTTCATTGGCTTCTGAGTCTGAGAGAAATGGGTTCAAATCCTGAATACG
TTCCTTATCTGTGTGATCTTAAGACTCATCATTTAATATTCTGAGTCAGT
TTCCTCCTCTATAAAACAAGAATCAGACGGGGCACAGTGGCTCACGCCTG
TAATCTCAGCACTTTGTGAGGCCAAGACGGATGAATCATCTGAGGTTAGG
AGTTCGAGACCAGCCGGACTAACATGGGAAAACdCCCGTCTCTACTAAAA
ATACAAAATTAGCCGGGAATGGTGGTGTACGCCTGCAATCCCAGCTACT
Human Genome Map 2q22.1 (2040 bp sequence)

(SEQ ID No. 79)

47A
TTGATTCATGGGATGTTTATGTGGATAATTCCTTTGAAATCCAGCTTGAT
TTATGAACAATCTTCTCTGCTCTATTGAGCCATTAAATCCAGAGTATTAG
TGCATTTGGAATACACAGAGATGATAATGACATCCAAAGAAGAGTCCAGC
AAAACTTATTTCCATGAGGACTTTTTCAGAGGGATGAAGTAACATTAGCT
ATACAGGTTAGCATTATAAGACTTCCCAAGTGTAGATGAGAATAATGGCA
ACTCTGTGGTCCTAAGGATGAATATTGCTCTGGAATATGCATTTTACACT
ATATGAAAGAAATTAGGATCGATATAAGCTCACTTATCTTTGCCTTATTC
CTCCTCATGTTGTTTTTGTCTAGATTGTCTCAGCCACTTGTTTTATTTTA
CTTAAATTTTAATTTCATCTTATTGTAAACCTCCATTCCTTCAGAAACAG
GTCAAGAACATGTCAATCTACCTAAGTGAATAACTAATATTAACAATTAA
ATAATAAATAGTACTGAATGAATATACGAATACAAGAATAAATAAAAATA
AAATGTATTACTTCATCGATGGATTTCCTAGTAGATGGGGAAACGGTGAG
AGGATATGAGCTTCAATAAGAAAAATGGTGCAATAAGGCAGAAGCAAATG
CCCAAAACAAATCAACACATTCACAATTTTTCCAAGGACCTGTCATGTAT
ATATTTTTTTCTTTTTTTAACCATTTGTGGCCCCTTTTTTTAACCATTTG
TGGCCCCTTTCTTATATCTCATTTCTCTCTTTTGTAAGGCTTCTGTGTTA
ATTGACAGCATGTTCAGATATAAATCCATCACAGGAATGTGATGAAATTA
GCCATTCAGACCCCTGATATTAAGAAATTCAAAGAAATGGATAGAGTATC
CAACCAGTGAGGAATTAAAGAAGAAAGAAGAGAAAGAGAAGGAGAGAGAA
ACTAGCTGTAAAGTTGGGATGGGTCGGGGGTGGTGAAGAAAACCAATTAT
TCATTGAAGGTGCCAGAAGGAAAATTGATGGCATGAATCCATAGCTTCTC
ACCATAAAGGTGAATAATGACACAGACACTTAGATTGGGGAATGAGAAAA
AAAAGGTGCATGCAAGGTTCTTCTATTTATATCTGATTAAGATATGAAAA
GAAAATGAGAGACTGGATTACTAAAGAAAAATTCCAGACAGTTAAGCAAT
TTTAGGAATGATTCATTTTAAGATATGGCCATCAATTATTTATAAGGGTT
AATAAATAGATTTATAAGCAAGAGGTACATGGAATCTAGAAATACATAAA
TGCTCTTCAATTATTTACAGCTCTGACAGTCATAACACATGAACTACTAC
CAAAAACACCATTTACTTGACTTTAAAATTTGCACCATAAACTATAAATG
GACCAGTTATGGAGCATCAGCCATTTGTAATGTGCCATGCAATATTTAAC
ATCAACTAAATGTGTTTTCACTAGCTGCTGACCACTTGGATTAATTTAAT
AAGCATGCCTAGTGCCTAATGATTTATTTGTGGGTAAATGATCATAACTA
TTTAATGGCCTTAATATTACAGATGTAATTCTGAAATAAAATATCATAAC
TTGGATTTAGTACATCCAGTTAAATAACAAGCATCGACATTTTTAAAAAA
TAATAAAAACAGTGGCCAGAAAAAGAAATTAAAGCACTTGCTAGTCATAT
GTCCCCATAGGTTTCCAGCTTCATATTGGCTTTATTTCTTTTTTTCCTTT
CATTTAGGTCACCCATTAATTTTCTTTCTTCATTTGCACACCCTCTTCCA
TTTCCTGTACTATCTTTTGTTCTAATCCTCTAGTAATTCCCCAGTGAGCT
CTCAGCTTCCAAGGGCACTCTATTTCTATTAAGCATGGCAGTCAACAAGT
GGAAATAGTCCTTGGTTGTCCTGCTTTCTGGGTGAATAGCAGAGTCCCTT
TGCATCACCTCAAAGACTCTGATTCTCATGATCCTCAGTCTGGTGCTGAA
TTGTGCTTTTGCTCATCCACACACATCCCCTACC
Human Genome Map 11q24.2 (2100 bp sequence)

(SEQ ID No. 80)

48A
ACTCAGCAATGGGTAGCTATACTTGAGATAATAGGCGGGATTTTATGTGC
AGCAATGTAGAGGATGCAGGGGCCCAGGATGGGCTGCCAGGTCTTCCAGA
GAGTGCTAAGGTATCCACCAAGGATCATGAATGTGAACAAGATAATGAAT
CACTGTCTACTTACTCTTTTGGAAAAGCTTCCATATCTCTGCCAATTGAA
TCACACTATAACCAGTCCCAGGCAATTCAGGATGACAAGTTCCACTTCGA
ACAGTTCTGGGAGTCATCCTGAGGGTCCCTGTGTATAGACATAAAAAGTT
CCATTTGTTCTTACACAGTGAAAATGACAGAACAAATATTATGGGGATTA
TGCCTGGGGAAAAAAAATCTGTCTCTGGATATTCCTGACACTATGGAGAG
AAAATCAGCAAAATTTAGAATCTTGGATCTCTTCCACTCACACTAGGATG
TTGTTTCTAGAAATCTCCCTGAAGTATGGTACTGACTCTTGGTGGTAAAA
GTGGAGAGGCTTAGAACTGAAATCTGGTCAGTAGAAGACTGAGGGTTAAA
AGTGGACGGTCAACCCATTGAATGAAGGCCTAGCAGGAAATAGAGAGACA
AAAATACAGGCATTAAGGGAATAATAGCTGAATAGTAATAATAATACATT
ATGTCAACAGCGGTGACAAAGGAAACACTCAATGTATTTATAGAGCTAAA
TAAACGGCAGATCTAGGTCCTACGTTTTGACTCTGAACAACCTTCTCGGT
TGGATTTTGCTTCTGCCTAAGGATTATTTTGGAAAGAGCTATTATTATCC
GTGATTTATCACGCTGCACTGGGGGGAACTCATACTTTCCACGGAGACAA
TTACTGAATTCTCACTGGAGGCGCTTAAAGGAGCCAGGACCTGTTCTGAG
GGTTCAGGTGGGAAAGGTGTGCCAGCAGGGGACTGCAGCCTGGCACCATG
GGACGTGTGTGCTGTTGACCACTTCTGTGCCCAGATCCCTCAGGCGCTTT
CTCATTAGATGCACCCTTCAATCTCCTGGTTATTGAAACAGGACTGGGGA
GAGGAGTTCACATTTATGGTGAGCCCATGCAAGAAGACCCTCCGACAGGT
GCCTGTCACCCCTGAGGAGTCACTGGTTGCAGCCCGTTCTGAAGTGTCAT
TGAGATAGAAACCAAGTCAAAGCCGTGGCCTAGAAAGAGAGTCTGGGCAG
AATTCTGCAAGCAGATTCTTTATTTGAGTAAGTATTCCTTGAAGAAGCCC
AGTTGTGCAGCTGTGTTTGGGTGGAGGTCATCAGAGGTTTAGAAAAAGAG
AGAAGTCATGGTTAATATTAGAAAAGAACTCTGAGAATCTGGAGGAAGGA
AAATGCATTACTAGTTCTAAGCAACAACTGTGGAATAAACAATGATAAAT
ACCGTATTAAATCTAAAGAGTTACGTTAATAGATAATAACAAGTAGGAGA
GCTAATAGCTAGCCATTAATACAGGCCAATTTATTATTTAAAACATTTAT
TAAGATTTAACAATAGTCAAATAATTTTTTTGTGAAACAGTTATTAAACT
GAATCTCTGCATACATTAATCAACTGATATTTATCATTCAGAATGTATCT
CATTATATCCAAAAGGGTGTGTGTATAGGCTTCAAAAACAAACTGGAAGA
TTTAAAATGAACTGTAGTTCATTTTTGCAAAGTGTAGATGTGTAAAGATT
ATTATGTTTGCCAGCTGGGTAGCCAGACAGTGAAGTGGACTTGTCTAATT
AGGAACAATCGCTGATAAATCAATTCTTTCCTTTTATAGGACAATTACAG
TTTGTGTGTATATGTGATTGTGTTTTAAATTCTAATTCGATTTTGTGCAT
TGTTCTGTAACCAAGTTAATTCTTTGAAGCCTTTTTAAATGGTACAAATT
TTCCATAAAATATAAATAGGTTTATTGCTGTTTTATCAGTCACGCAAATA
ATCCAAGATCCATCTATTCACATAATTCAGGCATTAACTGTGTATAATTA
CTCACATGAAGTCTTCAGTCTGGTTTACTATACGGAACCCCAAATATGAC
TTTAAATTGCTCCCCTCCTCTTTTCCTCTGTTATTTCTCTCCCTCTCTT
Human Genome Map 21q21 (2103 bp sequence)

(SEQ ID No. 81)

49A
TTGCATCATCTGACCTCTCTTCGAGTCCAAAGGACTGAGAACTAGAAGAA
CTACTGCTGTAAGTTCCAAAGTCCCAAGGCCCCTGAACTAAGATCTCCAA
TGTATGAGAGCAGGAAAAGATGGATATCCCAATTTAAGGAGAGAGAAAGA
GAAAATTTGCCCTTCTTGTTCTATTTAGATCCTCAAGGAATTGGAATGCC
CAACCACATTGGTGAGTGTGGGTCTTCTTTACTTATTCTACTGATTCAAA
TGTTAACAGATTCTGGAAACGTTCTCACAGATACACACAGAAATAATGTT
TTGCCAGCTCTCTGGGCAGCTCTTAGCCCAGTCAAGTTTACATATAAGAA
TAATCAACCCAGCTTTTTATAATCATCTTAGTATTTAATCAAGGAAATGA
TATCAGCTATCTACTACCACAAAAATAATTTTAAAAATTGGCTTATAAAA
GATGACTTAGTGGCCCTCAGCTGGGACAGCTTGTCACTATCCAACCAGCC
TTTAAATCCTTCCAAGAAGCAATCCTGGCTTGTTCTCCTGAAAACTGGGT
AGTTTTTCAAGAGATTGAGCAGAAGCATACAAGGCCTCCTGACACCTAGG
TGTGAGATGGGCACACAACCACTCCTGTCAAATTCTATTGGCCAAAGCAA
GTGACAAAGCCAATGCAGATTTAAGAGGTGGTGGAAAAAAACCCTAAAAA
TAGAAGTTGTTGAAAAGTCATATTTCAAAAGTCATTGGTATAAAGTAGTG
AAAAATTTGACATTTTTGCAATCAAGCTTATCAAACAATATTATCCCAAA
ATATAACAATACACTCAGTTTGCACACTTGTTTACCTTTTGCAAAACAGG
TAAGACAGTAGGACAAAGCAGGTGCTTTATGTTGTTTCAATCATTCAGGA
TTTGGACAGTTTGGATATTTTCTGTATCACTATAATTGATAAATACTCAG
ATGATTCTATAGTTAAGTTAGAATGGAAATTTTGGGTATAGTAACAAATA
CTACTTTAATTTAAACTTACATGTAAACAGTTTCCCTAAAGCAGTTAGAA
GTGTGACCATAAAAGTGAAATGGTTTAAATACATGCATTTACATCTGTCC
TAGAGTGATTAATGTAACTTTATTATAAAACTACTAATTTTGTGATTACA
TACACCCTTCCAAAGATACATTATACATTCCTATGTACACTCAAATATTA
TTTTTAAACTTCCATTCCAATCATTAAGTAGAAATGCATTTAAGAATCAT
GATTTTTTTAGAGTAAGTCTATAGGTGGTACTTTTATATTATAGATAACA
TTTCCTATACCCTTTCCACATAAACACAAGAACATTATGCTATAGATTGA
AAATTCCTGTAAACACTAAGCAGAGCTTTTGTACATAAACTTGTAAAAAC
TCTACATAAATGTATTCAGAAATACATGCTATTAAAATATTTTATTGTAT
ATTACTGTTTGGAAGTTTTCAGCTTAAATATTTTTATTTGATGATCAATA
AGATCTAGTATTAAATGGTCTTATTTATTAACCATTAAGTTAAATACATG
GAGAAATCCACTATGTCTTTTCCTCCAGCCTGTAAGTAACACAGGGTTGC
ATTTCTAATATTAACTAAGTTACATGTATTTTCCATTGAGAAGAGTGCTA
TCGAACTCATCCATGTTAATCACTCTTATGTGGAAAAGGCTAACATATAA
ATAAAAAAACTAGAAAATTTAAAAAAGGATAAAGAAAGAAGAAAAATGAA
CAGAATTTAACAGCAGTGCAACAGTAGTCTCTTCCTACCTTTCCTGGGCA
TCTTCCAATTTTATGGTGGTCTGATAAGCTTTCCAAAACACTTTGCTCAT
TTCCAGCACTGGACATTTACACTCAAGACTGCAGACTCGAGGAGTCACAC
ACTCAGCATCTTTTAGCTGTATGTTGTCAAGTTCAGACTACTCAAAGTGG
CATGTCTTTAAATTAGAATGTGTCAAGTGGGTCTAGTAACTGCACCGAAA
TATTTTAATAGTCATATTAATCATTAATAAGTCAGGAAACATGTTTTTCT
AATTTTCAGATCCCAATACACATGACTGATATGGTTTGCATCTGTGTCCC
TC
Human Genome Map 21q21 (2100 bp sequence)

(SEQ ID No. 82)

50A
AAGGTTTGGAAAGCTTAAGTCAAAATGTGTTGTTCATAAATACGGTCTGA
ATAATTTGAACATTTTCTGTTAATGGTATTTGTTCAACTATAATGATATT
TTCCAGCCAAGATATAATTGGCAATGTCAAAGTCACACACAGATGGGTAA
AATGGCCAATGTCTCTGGAAAATCTTGATAATAACTTTTTAGTATCTCTG
GTGCAAGGTCACTTAAATTCAGAAAATAGCACCCAAGGAAAAAATAGCCA
TATTCAAAAAAATAAGCTCCATATATTTAGATGTAGATATAAATTTGGGG
TGATTTATTTCTTATTAGACACTAATATTTTTTAAAACAGAGAATGACAA
ATAAGGAAATTTTGCAGTTAACTATGTCCTAATGAAAAAGGGTAGTAGTT
TTACAAGAAAGATATAATTCATCAAAAAGGCAGGGAAGCATTCAGACTAA
ACATTGAGTATGTTTGGAAATAATAAAAATTATTGTTTCTTTTACCAACA
TCAACAATCTTTTCAAATTAATTTATAAAACTGTCATCTCTGTTCACTAA
TTTTGAATTACTCATATTATTTTTAATTTTGAATACTTATAATATTACTT
ACTAATTTTTAATTAATTTTGATATACCTATATCACTGTTTTGAATTGAT
CTATGAATGATCTAGAAATGACTTTGCCTGTTTTTTTTTTTGACTCATGG
GTATTTACTTTTCATTAGGTAATTTTAATGTATTGTTAACTAGAAAAATA
AGATGAAGAAAAAAACATTTTAAATGCAAAATATAAATTTAAAGAACTTC
AAAAGAATAAAATTTCAGTTTTATGTCTTTCAAGTAAATTTGCTGTTTTC
AAAATTATTTTTTGTTACAAACCTATTTTATTTCAAAAAATATGCTATTG
TTTTTAACCTATAATTTTTAAATATCTGACAGCATTGTAGGACTTAAAGC
TATTAAATATATAAAGATATAATAGAACTTATTGGAAATATTCAAGGAAA
AACTAACATATTCTTTAAAAACATTTTAATTTTTAAATTCTATGTTAATT
GACTTTTTGATACATATTTTACTTTTCCTTCACTTCTTTTGTCAATTCTT
AAAAATGTCTTTCTTCATAATTTTTGGCAATTAGTTTTTACACTTTAATA
GCAAACATTGCCATAAAAGTGAAATTAAGCATTAATTAATTTTATGTCTG
CAGGCAGAGTGATTTCCTTAGGGAATCAATTTAATAGAGAGAACTATGTT
TGTACCTGGCAGGATATTCACAGAAATAAAATATTTATTGGCCATCTACT
TTGTTTAAGACCTCTTAACAAACCATAACTTATTAAAGCATAAAGTAACA
TACATAGTAAATACTTTTAAAATCTGTAAACAACTAATTCCTTTCTTCTT
GTGAAGTCTTGTTTAGATCATTAAAGTAATAGCAGATTTTCTCACAACAG
GTTTGTGAATATTGTCTGTTTAACATGAAAACTATAAAAAAATTAAAGAC
AATTGATATATATTTATTCAACTATGTCAACTCAAAGATGATCTGCAATT
GTTTTCTGAATAACTTATTAATAATGCTTAGGCCCCTTTGTTGAACATGC
TTTTATTTGTGTAAATAAGAATTCATTTAAAAATACATTGTACAACTTCA
ACACATTGTGTGTCCCTGAAGGTACTCTGAGATTTTGCAGTTATAGTATA
AATGAGACAAAACGGCAGAGAAAATATTCCCCATGTGTAATTCTTTCTAC
ATTTATTTCCCACATCAATCTCACAAGTGTTTTTATTTCACACTGATTGA
TATCATTGAGCACATACCTCAATATCTATTATCACAAAAACTATCATTAT
CAACAAGGACTTTAAAAAATATCTAAACATTATTATCTGGGTAGCAACTC
TATACTCCATTTTATCCATTAATTTTGTCTAATTAGTAAAGAAGTACTTA
TGGTAAAAACAAATTAAAAATAGTACAGAAAACATACTCCTGTATGCAAT
TATTACAAATATTTTATTTAGTTCCTATAAAGTATTTACATAGCTGAGAT
CACTATATAATATTATACTCATGTTACTTTATGTCCTAACTTTATATCA
Human Genome Map 11q23.2-q24.2 (2040 bp sequence)

(SEQ ID No. 83)

51A
CGGCTCTCCTGGCCTCGCGCTGCACATTCTCTCCTGGCGGCGGCGCCACC
TGCAGTAGCGTTCGCCCGAACATGGCGACACGGAGCAGCAGGAGGGAGTC
GCGACTCCCGTTCCTATTCACCCTGGTCGCACTGCTGCCGCCCGGAGCTC
TCTGCGAAGTCTGGACGCAGAGGCTGCACGGCGGCAGCGCGCCCTTGCCC
CAGGACCGGGGCTTCCTCGTGGTGCAGGGCGACCCGCGCGAGCTGCGGCT
GTGGGCGCGCGGGGATGCCAGGGGGGCGAGCCGCGCGGACGAGAAGCCGC
TCCGGAGGAAACGGAGCGCTGCCCTGCAGCCCGAGCCCATCAAGGTGTAC
GGACAGGTGAGCAGTTTTGCAACCCGCCTCCCTCCAGTTTTTTCCTCTCC
CTGCACTTCCTCACCCCCGCATCCATCCGTTGCAGTCGCCTCCTAGGTGC
AGGCACCACTGGGGACTTCCCGGCTTGCATTTGTTTTTTTCCTTCACGAG
TACAACCGTCAGCACTTGAATCGCATTGATCTTTCCTTCTTCCTGTCGAT
TTAGTAAACGTATTCCAGGTAACTCGCCGGGTGCAGTGCGTATTACCCCA
GGGTGTGTGCAGAGAGATGTAGTTTCCGGCAGGTATAGGAGGGGTGCAGC
TTCATTTTACATCTGGATAAAAAACGGGCTTTCTTTAGTGTATCATCAGT
TGGCAGTGGAGGCGAGCACCCTGCAGTTGCGGTACACTTACACAGAACAG
CACGAGGTGGGGGTTTCCACACTTAGCATTATTAGCACAATAAAAGTGGG
CAAACCTGAAAGCTTGTCGACTATCTCTGTACAGTCAGACAAGAGGTGTG
TGTATGTGTGTGCGTGTGTAAAGGCTGAATTTTTAATTTTTAATTTTTGG
CGAGCGTGTGAGATGCTCTCCATTCCTTCTTCCCCACCCTTCAAGATGCT
GACTCTCCCACCCCCGTCAAGATAACTTTATTTTGGAGAGGAATACCCCT
CATGGCACTTGGAGATTTGAAAGGACTGCAGGAAATTTGGTGGGCATTAT
TATTCTATAAGTGATTTATTTCTACCCAGGCAATAGGTTTATTAGATCAT
AAGTAACGTGAATTTCACTTTTATGGTCAGACTTACTGCGAGGAATTGCA
GATGGAGTTTGTAGGTTAGGATCAGCACTGGCAAAATTAATTTGACCGTG
TTATTGCCTCATGAGACTCCCAGTCCTGCAGTTAAGATTGACATCAGCAA
AAGTATAAGGTCGGTGGGGGAGAAAAAGTAGGACCAGAGGAGGGGGTAAA
TACACTTGTTTTCTAGAGTCAAATTGTTCCTTTTGAAGTAGAAATTATTA
ATAAAAGATTACCCTGAGTTCTGCCTTTTCTCACTAATTTCACTTTAGCC
ATTTCTTCAGGAAATACAGAGTTAAATGTTCAACCCTTGGATCCAGGACG
AACCTTGTAAACATATCACCCTATTGTGTCATTTTGTTGGTGAAGAAACT
GAAGCGTGGAATGGTGAAGTGACTAGTCCAAGGTCATACCGGGAAGGTGG
CCTGCTCTCTAGTTTTTGTCTGCATTGTCTCAGTGACCTTTGCTTGACTG
CAGTCACCCTGTCTTTATGCAATGCTGCTGAAATACCTCCTTTCTAAAAT
AAAATAGATCTGGTATAAAGGGGGAAAGGATGGTGGTGACTGGGTGGGAG
CGTTGGATTTCCCTCCACTATTGGTCCCTGGGCAAGAATGTGTGCCCCAG
GGCATGTAACTAATGGTGGCCACAGGCTGCAGGAACCTGCATGCTCAGTT
CCTCTTGGGCCCAGATCCTTGTCCCCCTGTCCCCACCCCATATGACAAAT
ATGTGTATGAACAAAAAGAAGTCATCAAGGTCCTTGCTCTTAACAGCGAC
ACCAGCATGGGGCTGATGGAGGGTGGGAGAAGGAGGAGGAGTGGCCCACT
TCTTCATTGGGCCTCCGCAGTCAGCCCAGCTCTGCTGTGCTCTTGAATCA
GCATTCTGGGAACTGGGAGTTGGGGGCTGGTGGGAGACAA
Human Genome Map 8p11.2 (2100 bp sequence)

(SEQ ID No. 84)

52A
ACAAAAGGCAAATTGGTGTCTCTGTCCTGGAGTCCTTACTCCTCATCTTG
TGCTTAGACATGAAATTACACATCTCCAGCCTTGGGATTCCAGGACTTAC
ACCAGTAGCATGCCTATGTTCTAAGGCCTTTGGCCTGGGACTGAGAATTA
CACCATCAGCTTTTCTGGTTCTAAGGCTTCTGGACCTGAACTGAGCCATG
CTACCAGTATTTCAGGATGTTCAGCTTGCAGATAGCCTGTCGCGGAACTT
CTCAGCCTCTAGAATCACATGAGTCAATTCCCCTAATAAATCTCCTTTTA
TCTATCTGAACATCTCTCTTCATCTCTCCATCCATCCACTCATGTGTCCA
TCCATCCATCCATCTATTGCTATCTATCTATCCATCCATGCATCCATCCA
TTCAACCATCCATCCACCCATCCATCCATCCCTGTGCCATCTATATCTAT
CTATCTATATATCTATCTATCCATGCATCCATCCATCCATCTATCCATCT
ATCCATCCATCACTATCTATCCATGCATCCACCCATCCACCCATCCATCC
ATCCATCCATCCATCCATCACTATCTATCCATCCATGCATGCATGCATCC
ATCCATCCATCCATCCATCCATCCATCCATCCATTTATCGCTATCTATCT
ATCCATCCATGTATCCATCCATCCATCTGTTCATCTATCACTGTCTATAT
ATCTATGTATCTATCTATCCATCCATCCATGCATCCATCCATGCATCCAT
GCATCCATCTATCACTATCCATCCATCCATCCATCCATCCATTCATCCAT
CTATCTGTCTTCTACCTACCTACCTATCTAACTCTCTGGAGAACTCTGAC
TAATAAACTAGCTTTAAAACATGTTATTCTCTCTCTGCAATGTCTATTGC
TTTATCTTCAGGAACATTCCACACATCCTGTAAGACTTCAGTTAAATTAT
CTCTCTGTTTCTTCTCCAATCATCCTCTGCCTTCCCTAGTCTCCTAACGT
ACTTTGTACATCTGTCACAAACCCCTCATCATATTTACTGTAATTTTTTT
CCTACAGATTTGGATAGGAATTGAGCCATTTTTTTAATTTCACTTTTATG
GTTGTTACAAATAAAAGAGCAAGCAGGCCCCTCACTGTAATTCACCTGTA
TTTGCATTTAACTTATTAACCAAGGCATACTATTTCAAATAATCTAATAT
AGTATTTCCTATTTAATAACCAAACATACAGAACAGTTCCAAGCACATGT
AACCATGTGATACATTTTCCTCTTTGAATAATAAATATATTTCTTATAAT
TAATATGTGATAAAATTGCAATATTTTTAATCTCCTACATCCTTCTCTTT
TAACAGGTTTCCTTATCAACTGGTTCCTATCTCACGGGGTTGTTGCAGAG
ATGAGGAAAAAAAGTATTCTATTGGTTCATGCATCTCAAAATAGGCAGAT
TCTTTTCTCTGCTTCTTCCTTCATTGGCTCAGGTGTGGAGTGCTTCTCCC
AATTATATGTGCCAGCCTTGGTATGTTCTCATTGCTGTACCACACTGCCT
GAGACATCCAAGACCACATCTTCCTTTGGGGGCACATTGGACCTTTGTCA
TTGGCACTGGCAGGGAAGCTTTTATTTCACCAGGTCTAAGGCAATTCTTC
CAAAAAAATCCCAAATAGTGAAAGAATTGATTTATTCTTCTAATATTTAA
GCAAATGTAAAAAAAAAGTTACATTAGTTATGTTTTTTTCAGATTTTGGA
TCAGTGAGACTTCATTAAAACACTTTGAGGTTATAAAGCAAGTAATTTTT
GTTTCCAGAAAAGTTAGTTTCCTTTGGCTGAAGGGACATCTCTATGCAGG
CCAGATCAAGACAAAAATAACTTTTAAGAAGGGAAATGAGGGAATGGAGT
TTGGAAAACATAAATCCCACAGCAAAGTACGTCACCAACAATAAGAGTCA
TCTCTTTCACAGAGGCCTTTCCTAGAAAAGCCCTGACAGACTAGGAGTCC
AATCTTCGGCTCCCATAGCACCCATGCCTGCTTCCACTCTGGAGCTTACT
ACTTTGCGTTGAAATTAATTTTTACATGTCTATGGCTTCTATTACAAA

However since the in silico search was based on the H1 PSE consensus and considering that it was used as query allowing only the first and the last bases to be different in the targets, it can reasonably supposed to have identified only those promoters whose, structure is very similar to that of H1 (for sequences used as query see materials and Methods section). This is further supported by the fact that out of H1 no other previously known Pol III Type III promoters were found in our PSE-based collection. Therefore this finding together with the observation of the large sequence divergence among the PSE consensus sequences of U6, 7SK and H1 suggests that the use of a degenerated PSE consensus as query (most likely derived from a bioinformatic analysis of several known Pol III Type III promoter consensus elements) would bring to light a considerably higher number of novel PSE-dependent transcription units in the human genome that would better clarify the likely impact of this effect at genome scale.

In order to further characterize in silico the novel transcription units we arbitrarily assumed as transcribed the sequence stretch starting from the 21^{st} nucleotide downstream the predicted TATA box. In addition a 4×T repeat was considered as a Pol III transcription STOP signal although events of “read through” are possible and most likely affected by sequence context features [19, 20]. Although it has to be emphasized that the transcribed region of each element of this collection needs to be experimentally determined case by case (possibly in the context of its target gene of regulation), based on their in silico characterization we selected 33 novel transcripts to be subjected to additional analysis.

In order to test if a common secondary structure could be a hallmark of the novel molecules an in silico analysis of their secondary structure was performed by mfold algorithm (http://www.bioinfo.rpi.edu/applications/mfold/rna/form1.cgi) [21]. Results showed that although hairpins with short stems (5-7 base pairs) were frequent no shared secondary structures were recurrent indicating that a peculiar molecular organization is not the common hallmark of this set of non-coding molecules. Interestingly, although their averaged free energy (δG) was extremely variable (−42.7±41.2) four transcripts (11A, 20A, 21A, and 29A) showed a δG value significantly lower than all the others (δG<−100). A statistical analysis of such δG differences was performed bringing to light a group of transcripts (11A, 20A, 21A and 29A) whose δG is significantly lower then expected (Student't TEST, 33 degrees of freedom, α significance level=0.1 corresponding to a P-value of 0.0001) thus keeping in line with their physiologically functional molecular organization (FIG. 8).

In order to assess if the pool of transcription units was prevalently constituted by repeats such as retroposons we analyzed all the transcripts by Repeat Masker algorithm [22] evidencing that: i) only 2 out of 34 (5.9%) are Short Interspersed Nucleotide Elements (SINEs) such as 21A and 29A that were marked as AluJb elements. ii) three of them (8.8%) are part of Long Interspersed Nucleotide Element (LINE) such as 24A, 37A, and 38A. iii) two (5.9%) contained a MIR (17A and 40A) and iiii) three contained different types of Long Terminal Repeats (30A, 32A and 44A) (Table 2).

TABLE 2
Sequence analysis by RepeatMasker Web Server (available at:
http://www.repeatmasker.org/cgi-bin/WEBRepeatMasker)

| | Seq. | Rep. | | |
|---|---|---|---|---|
| Seq Name | Length | Length | Position | Type |
| 11A | — | — | — | — |
| 12A | — | — | — | — |
| 14A | — | — | — | — |
| 17A | 159 | 115 | 1-115 | Mir (MIR3) |
| 19A | — | — | — | — |
| 20A | — | — | — | — |
| 21A | 333 | 307 | 18-324 | SINEs (AluJB) |
| 22A | — | — | — | — |
| 23A | — | — | — | — |
| 24A | 406 | 406 | 1-406 | Line L1 (L1MC) |
| 27A | — | — | — | — |
| 29A | 360 | 286 | 74-359 | SINEs (AluJB) |
| 30A | 158 | 135 | 1-135 | LTR/MALR (MLT1G3) |
| 31A | — | — | — | — |
| 32A | 140 | 140 | 1-140 | LTR (LTR7) |
| 33A | — | — | — | — |
| 34A | — | — | — | — |
| 35A | — | — | — | — |
| 36A | — | — | — | — |
| 37A | 50 | 50 | 1-49 | LINE (L1M4) |
| 38A | 357 | 348 | 1-348 | LINE (L1M3) |
| 39A | — | — | — | — |
| 40A | 484 | 220 | 253-472 | MIR (MIR b) |
| 41A | — | — | — | — |
| 42A | — | — | — | — |
| 43A | — | — | — | — |
| 44A | 218 | 52 | 66-117 | LTR/MALR (MLT1M2) |
| 45A | — | — | — | — |
| 47A | — | — | — | — |
| 48A | — | — | — | — |
| 49A | — | — | — | — |
| 50A | — | — | — | — |
| 51A | — | — | — | — |
| 52A | — | — | — | — |

Placing results in the appropriate context (such as considering that Alus, LINEs and MIRs constitute about 15%, 30% and 1-5% of the human genome respectively) one should expect a higher frequency of repeats in this novel pool of sequences. In addition we observed that no more than three of the repeats-containing elements are ascribed to the same class of molecules. Altogether these observations evidence that the novel PSE-dependent transcripts are not associated to a specific class of repetitive sequences scattered throughout the human genome but instead they constitute a novel eterogeneous set of Type III promoter-driven elements.

When these non-coding sequences were used to challenge the human genome database (BLAST Analysis) results showed that 7 were internal to known or predicted protein-coding genes, 4 being in antisense and 3 in sense configuration. Interestingly, most of the novel sequence elements not mapping in coding regions shared a high sequence homology (˜80%) to a Pol II transcript/EST that maps in a different locus (Table 3). Such homologies reached much higher values (often about 90%) if only parts of the putative transcripts were considered. In fact, no ESTs entirely containing one of our transcription units were found so that if a sense/antisense-based regulation would occur it should be related to parts of the ncRNA sequences while the other part could have structural properties that facilitate this regulatory action (perhaps binding specific structural proteins). Based on these observations, a novel control mechanism of gene expression could be postulated where Pol III (or Pol III-like) elements act as trans-locus antisense of their homologous protein-coding RNAs. In this model the Pol III co-genes in antisense configuration with respect to one (or more) specific target gene could regulate its expression either by interfering with its mRNA maturation (if the homologous region is internal to an intron) or by inhibiting protein translation (if the homology is associated to an exon).

TABLE 3

| | | | | | | BLAST | | |
|---|---|---|---|---|---|---|---|---|
| | | | Genomic | Blast Human | | Human | | |
| Tr. Unit | Tr. Length (nt) | Hum. Gen. Map | Contig | Genome | gi (gen. ident.) | ESTs | gi | e-value |
| 11A | 344 | 14q22.1 | — | H1 RNA | — | — | — | — |
| 12A | 141 | 2p24.3 | RP11- | — | 51460874 | DB275493 | 83216976 | 2.00E-12 |
| | | | 98I18 | | | | | |
| 14A | 148 | 3p12 | RP11- | — | 21206095 | — | — | — |
| | | | 206J21 | | | | | |
| 17A | 159 | 9q22-q31 | RP11- | GPR51 (intron 3) | 51467683 | — | — | — |
| | | | 349P17 | Sense | | | | |
| 19A | 148 | 3p12.3 | RP11- | — | 19774315 | — | — | — |
| | | | 206J21 | | | | | |
| 20A | 547 | 14q22.1 | R218E20 | — | 11611180 | — | — | — |
| 21A | 333 | 8q24.1 | RP1- | CENPF (Intron | 22657510 | AA737281 | 2767556 | 1.00E-31 |
| | | | 316L14 | 7,14,18) Antisense | | | | |
| 22A | 235 | 6q16-q21 | RP11- | — | 10045412 | AA361955 | 2014276 | 6.00E-29 |
| | | | 487F5 | | | | | |
| 23A | 200 | Xq21.3 | RP13- | — | 6855342 | — | — | — |
| | | | 258015 | | | | | |
| 24A | 406 | 12q21 | RP11- | — | 9957971 | DA811538 | 81279558 | 6.00E-15 |
| | | | 997P16 | | | | | |
| 27A | 91 | 7q22 | CTA- | — | 2341013 | — | — | — |
| | | | 369K23 | | | | | |
| 29A | 360 | 11p15 | AJ400877 | ASCL3 (intron 1) | 8052236 | BX645799 | 34480132 | 2.00E-36 |
| | | | | Sense | | | | |
| 30A | 258 | Xp11.4 | RP11- | — | 50582666 | DA496935 | 80536970 | 3.00E-05 |
| | | | 157D23 | | | | | |
| 31A | 231 | 12q21 | RP11- | — | 21039699 | AW303617 | 6713306 | 2.00E-19 |
| | | | 743I10 | | | | | |
| 32A | 140 | 17q21 | MCK41 | — | 75875068 | CA310957 | 24529055 | 9.00E-73 |
| 33A | 210 | 1q32.2 | RP11- | — | 21622744 | — | — | — |
| | | | 465N4 | | | | | |
| 34A | 33 | 5q15 | RP11- | — | 21281496 | — | — | — |
| | | | 274E7 | | | | | |
| 35A | 351 | 8p11.2 | RP11- | — | 28565756 | BF995135 | 12401458 | 5.00E-21 |
| | | | 1147M13 | | | | | |
| 36A | 122 | 3p12 | RP11-564- | — | 20334518 | — | — | — |
| | | | P9 | | | | | |
| 37A | 49 | 14q13 | RP11- | — | 37550867 | BM724961 | 19046292 | 4.00E-10 |
| | | | 192K2 | | | | | |
| 38A | 357 | 4p15.31 | RP11- | KCNIP4 (intron 1) | 19807889 | BF475563 | 11546390 | 8.00E-17 |
| | | | 19D21 | Antisense | | | | |
| 39A | 76 | Xp11.3 | RP5- | FLJ22843 (intron 10) | 9581533 | — | — | — |
| | | | 1158H2 | Antisense | | | | |
| 40A | 484 | 11p15 | RP11- | — | 27413210 | BG570298 | 13577951 | 4.00E-04 |
| | | | 265F24 | | | | | |
| 41A | 79 | 2q31 | RP11- | — | 15668089 | — | — | — |
| | | | 12N7 | | | | | |
| 42A | 122 | 3p12.3 | RP11- | — | 20334518 | — | — | — |
| | | | 564P9 | | | | | |
| 43A | 65 | 4q34.3 | RP11- | — | 18129587 | — | — | — |
| | | | 43303 | | | | | |
| 44A | 218 | 4q13.3 | RP11- | — | 18464317 | — | — | — |
| | | | 401E5 | | | | | |
| 45A | 78 | 4p14 | RP11-1I10 | APBB2 )intron 1) | 18450176 | — | — | — |
| | | | | Antisense | | | | |
| 47A | 48 | 2q22.1 | RP11- | — | 16950374 | — | — | — |
| | | | 745P9 | | | | | |
| 48A | 405 | 11q24.2 | RP11- | — | 32188045 | — | — | — |
| | | | 168K9 | | | | | |
| 50A | 156 | 21q21 | AP001675 | — | 7768691 | — | — | — |
| 51A | 273 | 11q23.2 | RP11- | SORL1 (intron 1) | 14517581 | BG698692 | 13966211 | 8.00E-04 |
| | | | 730K11 | Sense | | | | |
| 52A | 142 | 8p11.2 | RP11- | — | 28565756 | — | — | — |
| | | | 1147M13 | | | | | |

### 21A as Co-Gene Experimental Model

To test our hypothesis we selected one of the novel transcription units (here referred to as 21A) that maps in 8q24.13. If aligned to the human genome it shows several homology hits among which the highest were associated to multiple intronic regions of Centromeric Protein F (CENP-F; 1q32-q41) (Acc. N° NM016343) [23] thus constituting its putative natural trans-chromosomal antisense (FIG. 1A, B, C). Although similarly to all the 7SL/Alu-derived elements 21A is expected to be primate specific [24] an evolutionary conservation analysis was performed aligning its sequence with the mouse predicted CENP-F gene. No significant similarities were found indicating that in rodents a putative CENP-F antisense regulatory role, if any, would be associated to a different class of noncoding elements. Interestingly, in spite of its high sequence similarity with other human Alus, 21A lacks the Alu-specific intragenic consensus elements needed to promote its Pol III transcription such as the blocks A and B [25]. This was a further clue pointing toward a 21A transcription driven by an extragenic Type III promoter.

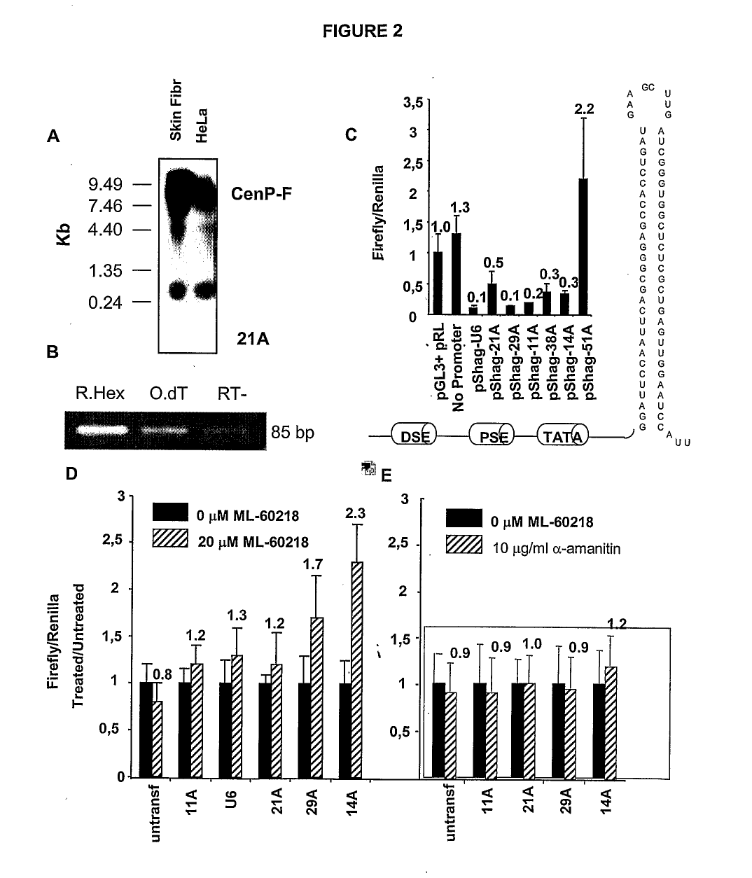

To check for 21A expression in cultured cells, we performed Northern blot analysis on total HeLa cell RNA using a 21A dsDNA probe. Two positive bands were detected: one corresponding in size to the expected 21A transcript (−300 nt), and the other one corresponding to a high molecular mass transcript (as expected for CENP-F mRNA) (FIG. 2A). However, considering that the 21A double-strand cDNA probe would detect transcription of 21A-similar Alus from multiple loci we also amplified a 21A-specific cDNA from total RNA samples, extracted from skin fibroblasts and four tumor cell lines (293T, LAN5, HCT, HeLa), by random hexamer-based RT-PCR in order to better identify a 21A-specific transcription product (FIG. 2B). The DNA band obtained was then purified and sequenced evidencing that the amplification product was the expected 21A. In addition, to better assess 21A transcription we fused its promoter to a luciferase silencer hairpin and co-transfected this construct with a plasmid expressing luciferase. Results showed a halved luciferase activity 48 hours after transfection thus demonstrating an efficient transcription directed by 21A promoter. In the same experiment a set of five novel promoters from our collection were tested demonstrating an active transcription of the hairpin promoted by four of them (FIG. 2C). These data support the conclusion that the majority of the novel putative transcription units is under the control of active extragenic Type III promoters.

### Pol III-Dependency of the Novel Transcription Units

The same experiment as above was repeated after 24 hours of cell treatment with ML-60218, a cell-permeable indazolo-sulfonamide compound that displays broad spectrum inhibitory activity against RNA Polymerase III [26]. Results showed an efficient luciferase-silencing activity in the absence of the Pol III inhibitor (as evidenced by a decreased luciferase emission) while after treatment with ML-60218 the luciferase signal was increased (FIG. 2D).

Altogether, these results evidence a decrease in hairpin synthesis of the novel transcription units as consequence of the reduced Pol III activity according with their Pol III-dependency of their transcription.

### 21A Acts as CENP-F Regulatory Co-Gene Modulating its Expression at Post-Transcriptional Level

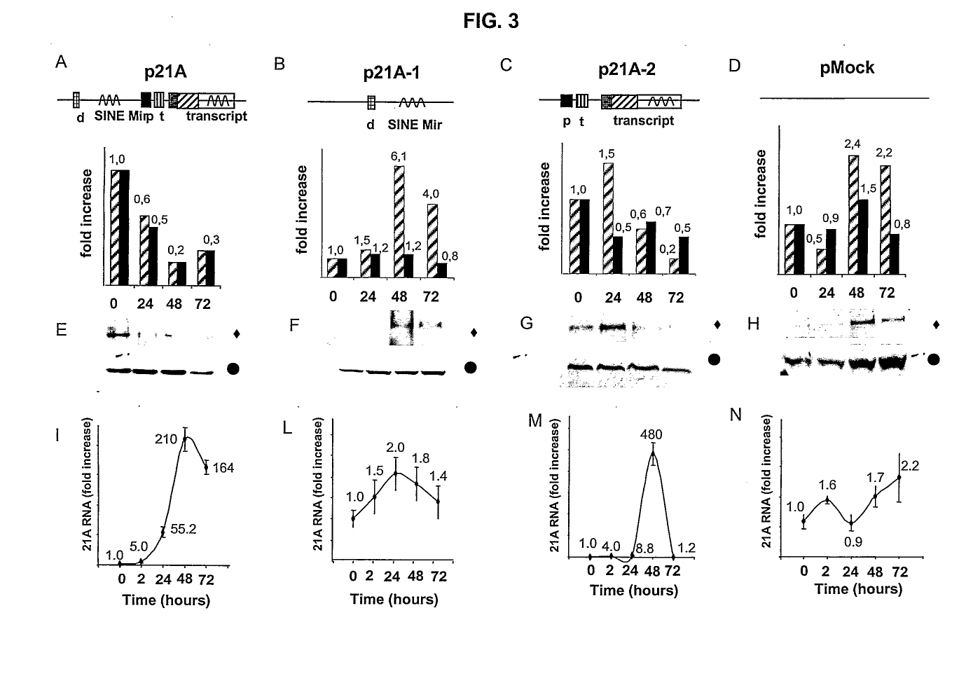

To test whether the 21A transcript acts as an antisense inhibitor of CENP-F expression we measured by Western analysis CENP-F protein level in HeLa cells transiently transfected with four different 21A constructs carrying: i) the whole 21A region containing both DSE and PSE elements (p21A), ii) its upstream moiety, that contains the DSE and a MIR element (p21A-1), iii) the novel Pol III Type 3 transcription region (that includes an Alu Jb module) (p21A-2) and iiii) an empty vector as Mock control (pMock). Starting at 24 hours from transfection of the whole 21A region, inhibition of CENP-F accumulation (followed by a rapid degradation) was observed. Such inhibition was specifically associated to constructs expressing the 21A RNA (p21A, p21A-2) while the MIR element in the upstream moiety of the fragment (p21A-1 construct) was ineffective (FIG. 3 A-D). In this context it has to be noted that a slight delay of 21A-2 inhibitory action if compared to the immediate CENPF decrease determined by 21A has been observed suggesting that a more detailed mutation analysis of 21A promoter could bring to light further Type III promoters regulatory regions. In order to measure the occurrence of 21A transcription in transfected cells we analyzed by Real Time quantitative RT-PCR its RNA level in all the samples. As expected a very high amount of 21A transcript was detected in p21A and p21A-2-transfected cells (210 and 480-fold respectively at 48 hours from transfection) while the 21A RNA content of samples transfected with pMock control plasmid and/or with a construct containing the promoter lacking the transcribed region (p21A-1 construct) were essentially stable showing a very low basal level of 21A expression in untransfected HeLa cells (FIG. 3 E-H). All the PCR products were analyzed in their dissociation curve showing a single characteristic pick (at 78/79° C.) in p21A/p21A2-transfected samples significantly reduced in pMOCK/p21A-1. On the contrary the cells transfected with the two control plasmids (pMock/p21A-1) showed a dissociation pattern characteristic of an eterogeneous population of molecules (FIG. 30). Again these results confirmed an active synthesis of the exogenous 21A ncRNA transcript in p21A/p21A-2-transfected samples that was strongly reduced at a very low endogenous basal level in the samples lacking the transcript region (pMOCK/p21A-1). As a consequence of 21A very active transcription the level of CENP-F mRNA (as determined by Real-Time RT-PCR) was significantly decreased in p21A/p21A-2-transfected cells while no major CENPF mRNA variations were observed in pMOCK/p21A-1-transfected cells (FIG. 3A-D). Altogether these results evidenced an inverse correlation between 21A transcription and CENPF expression. Therefore, considering the high homology level between 21A transcript and three CENPF hnRNA intronic portions and in the light of the above results (obtained either at protein level as well as at RNA level) we suggest a mechanism of antisense inhibition of CENP-F mRNA maturation by the 21A transcript.

### 21A Overexpression Specifically Inhibits Cell Proliferation in Humans

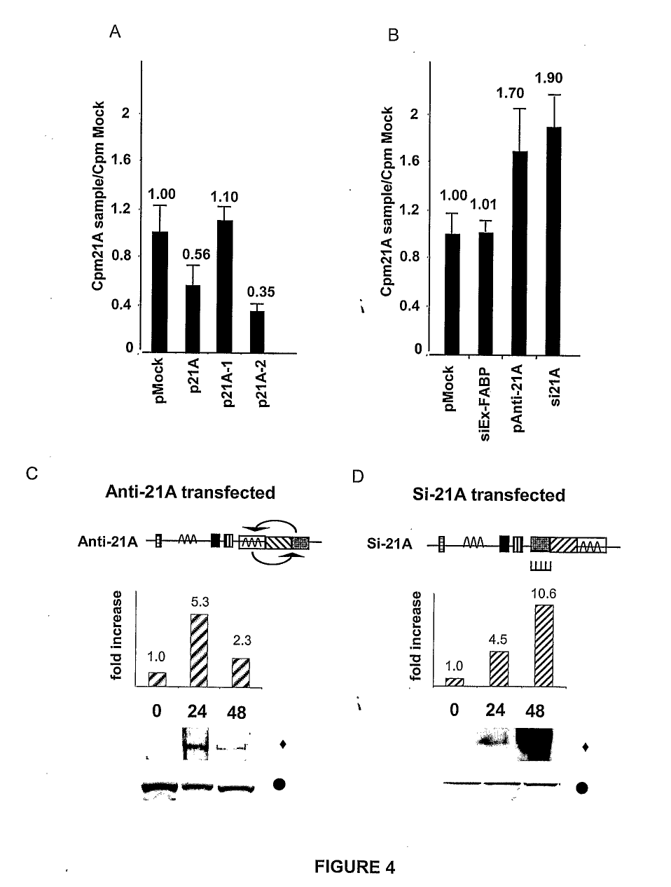

Given the central role of CENP F in mitosis we tested the effect of ectopic 21A expression on cell proliferation. By measuring [3H]-thymidine incorporation we evidenced a dramatic arrest of cell proliferation after 48 hours in 21A-transfected cells. Again, the effect was specifically associated to the downstream 21A transcribed region (p21A/p21A-2 constructs) while transfection of the MIR-containing upstream moiety (p21A-1 construct) did not alter cell proliferation (FIG. 4A). Although at the present state we cannot exclude a contribution to this effect by Alus from other loci, this experiment evidence an inverse correlation of 21A transcription and cell proliferation that is in accord with the inhibition of CENPF synthesis here demonstrated.

To further support the antisense role of 21A we transfected Hela cells with a construct expressing the transcript in antisense configuration (here referred to as pAnti-21A) thus quenching the activity of the endogenous 21A molecules. Results showed an increased cell proliferation 24/48 hours after transfection. Similar results were obtained when a 21A-specific siRNA expressing construct was transfected in HeLa cells while the negative control sample (cells transfected with an unrelated chicken-specific siRNA) maintained a cell proliferation rate similar to that of pMock-transfected cells (FIG. 4B). In both the experiments an increased CENP-F synthesis was detected together with the concomitant 21A-RNA decrease in Anti/si21A-transfected cells, as evidenced by Real-Time RT PCR (FIG. 4 C,D). As shown in these experiments CENPF modulation and 21A RNA decrease were analyzed only at 0, 24, 48 hours after transfection rather then at 0, 24, 48 and 72 hours as in the previous experiments. In fact, the proliferation increase that follows to 21A downregulation brings in advance the cells at overconfluence so that the effect that we would measure on CENPF synthesis at 72 hours after transfection would be strongly biased by this technical limitation.

These data suggest that the decreased amount of 21A transcript consequent to its siRNA-mediated silencing, as well as its suppression by antisense technology specifically increase CENP-F synthesis thus keeping in line with the proposed role of 21A as CENP-F regulatory co-gene. In addition, it has to be considered that the increased proliferation rate here observed supports the idea of a widespread regulatory action of 21A that may control at post-transcriptional level the expression of several target genes similarly to what has been proposed for miRNAs [27].

### The 21A Regulatory Effect is Human-Specific

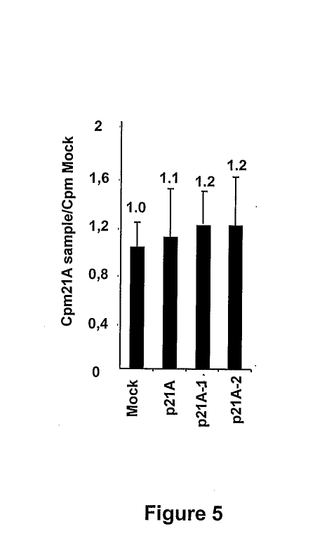

Considering that a 21A-driven cell proliferation inhibition is expected to be primate specific (Alu sequences were not found in other mammalian orders) we tested for its eventual occurrence in mouse. In fact, this would keep in line with an unspecific effect of 21A on cell proliferation may be due to the activation of a more general biological process such as most likely the interferon response (an antiviral cell reaction shared by all mammals) rather then a specific multilocus 21A regulatory action. As expected results showed that after transfection of p21A, p21A-1, p21A-2 and pMock the murine fibroblast NIH 3T3 cells did not show any proliferation decrease as assessed by [3H]-thymidine incorporation (FIG. 5). Therefore the 21A specie-specificity of action together with its inability to cause an unspecific cell reaction that leads to a proliferative blockade in mice further strengthen a 21A-specific (perhaps multilocus) regulatory role.

### 21A is a Key Factor of Cell Proliferation Control

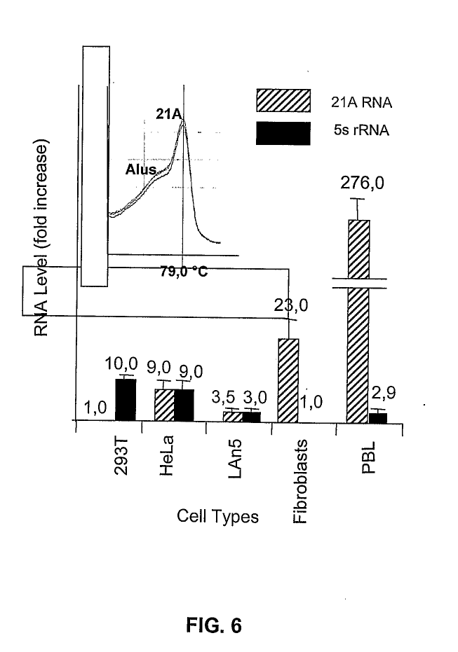

As demonstrated by transfection experiments 21A overexpression is inversely correlated to cell proliferation. According with this finding its expression is very low in fully proliferating HeLa cells. Therefore in order to further demonstrate the inverse correlation between the endogenous 21A expression and cell proliferation we analyzed by quantitative Real Time RT-PCR its transcription level in different cell types with various proliferation potential. Results showed that three immortalized/fully proliferating cell lines here analyzed (HeLa as cervical adenocarcinoma; 293T as renal epithelial adenovirus transformed cells; LAN5 as neuroblastoma) the level of 21A transcription was very low if compared to the unproliferating/resting PBL cells (such as peripheral blood lymphocytes) in which a 276-fold increased 21A transcription was evidenced. I the same experiment, according with an inverse correlation between endogenous 21A transcription and the cell proliferation rate, the 21A RNA level in primary skin fibroblasts (whose proliferation rate is significantly lower than that of the tumor cell lines here analyzed) showed a 23-fold increased if compared to 393T cells and a very low expression level if compared to the resting/unproliferating PBL (FIG. 6). Again the dissociation curve analysis of 21A amplification product showed in PBL a pick at 78-79° C. characteristic of a single specific molecular specie that resembled the one obtained in 21A/21A-2 transfected cells (where the amount of 21A transcripts was strongly increased) although a slight shoulder, most likely due to a cross-amplification of other very similar transcripts, revealed a detectable endogenous Alu transcription background (FIG. 6). Altogether these results evidence a very active 21A transcription in PBL/resting cells that furtherly strengthen the idea of 21A as a novel key factor of cell proliferation control.

In order to check if the endogenous 21A over expression in unproliferating cells was related to a widespread increased RNA polymerase III activity rather then a 21A-specific activation we measured by Real Time RT-PCR the 5s rRNA expression level in the same samples. The results showed no direct correlation between 5s rRNA expression and the cell proliferation rate variations evidencing that the 21A over expression in resting cells was the consequence of a 21A-specific transcription activation rather then a more wide, unspecific increase of Pol III activity (FIG. 6). Altogether these results suggest an unexpectedly specific expression regulation of 21A promoter (related to the cell proliferation state) that needs to be investigated in detail.

### CONCLUSIONS

We here propose that the non-coding fraction of the human genome includes a larger than expected number of ncRNA genes controlled by DSE and PSE promoter elements. Due to their promoter structure, a number of these genes is likely to be transcribed by Pol III. We refer to them as co-genes since they could specifically co-act with a protein-coding Pol II gene. Given the very high sequence homology between Pol III and Pol II transcript pairs and in the light of the results we have obtained investigating the regulatory activity of 21A transcription unit, we propose that a large part of these novel elements may act as antisense inhibitors of protein translation and/or mRNA maturation although some of them (those whose homology with the Pol II target gene is in sense configuration) could play a role in gene expression regulation with different mechanisms. Altogether these findings provide evidence for the existence of a ncRNA gene set associated to PSE/DSE-containing promoters, whose products co-act with a corresponding set of protein-coding targets.

In conclusion, this study provides i) a collection of novel non-coding transcripts to be investigated for their potential regulatory action with respect to Pol II target genes ii) a novel source of PSE-dependent promoters useful for the identification of common regulatory regions specific for this type of promoters, iii) a novel class of molecules involved in the RNA gene expression regulatory mechanisms iiii) a novel transcript (21A) whose intriguing role in tumor cell proliferation control would need to be investigated in detail in the context of cancer studies.

### REFERENCES

1. Reis E M, Nakaya H I, Louro R, Canavez F C, Flatschart A V et al. (2004) Antisense intronic non-coding RNA levels correlate to the degree of tumor differentiation in prostate cancer. Oncogene 23(39):6684-92.
2. Yelin R, Dahary D, Sorek R, Levanon E Y, Goldstein O et al. (2003) Widespread occurrence of antisense transcription in the human genome. Nat Biotechnol. 21(4):379-86.
3. Dahary D, Elroy-Stein O, Sorek R (2005) Naturally occurring antisense: transcriptional leakage or real overlap? Genome Res. 15(3):364-8.
4. Mattick J S (2004) RNA regulation: a new genetics? Nat Rev Genet. 5(4):316-23.
5. John S. Mattick and Igor V. Makunin. Non-coding RNA. Human Molecular Genetics, 2006, Vol. 15, Review.
6. Ambros V. (2004) The functions of animal microRNAs. Nature 431(7006):350-5.
7. Lee Y, Kim M, Han J, Yeom K H, Lee S et al. MicroRNA genes are transcribed by RNA polymerase II. (2004) EMBO J. 23(20):4051-60.
8. Carninci P, Kasukawa T, Katayama S, Gough J, Frith M C, et al. (2005) The transcriptional landscape of the mammalian genome. Science 309(5740): 1559-63.
9. Levanon K, Eisenberg E, Rechavi G, Levanon E Y. Letter from the editor: Adenosine-to-inosine RNA editing in Alu repeats in the human genome. EMBO Rep. 2005 September; 6(9):831-5. Review.
10. Rotem Sorek, Gil Ast and Dan Graur. (2002) Alu-Containing Exons are Alternatively Spliced. 12: 1060-1067 Genome Res.,
11. Am J Hum Genet. 2006 July; 79(1):41-53. Epub 2006 May 3. Human Genomic Deletions Mediated by Recombination between Alu Elements. Sen S K, Han K, Wang J, Lee J, Wang H, Callinan P A, Dyer M, Cordaux R, Liang P, Batzer M A.
12. Biochem Biophys Res Commun. 2000 Aug. 11; 274(3):641-8. The 3′ UTR of human MnSOD mRNA hybridizes to a small cytoplasmic RNA and inhibits gene expression. Stuart J J, Egry L A, Wong G H, Kaspar R L.
13. Liu W M, Chu W M, Choudary P V, Schmid C W. Cell stress and translational inhibitors transiently increase the abundance of mammalian SINE transcripts. Nucleic Acids Res. 1995 May 25; 23(10):1758-65.
14. Smalheiser N R, Torvik V I. Alu elements within human mRNAs are probable microRNA targets. Trends Genet. 2006 Aug. 14.
15. Schramm L, Hernandez N (2002) Recruitment of RNA polymerase III to its target promoters. Dev. 16(20):2593-620.
16. Myslinski E, Ame J C, Krol A, Carbon P (2001) An unusually compact external promoter for RNA polymerase III transcription of the human H1RNA gene. Nucleic Acids Res. 29(12):2502-9.
17. Lobo S M, Ifill S, Hernandez N. cis-acting elements required for RNA polymerase II and III transcription in the human U2 and U6 siRNA promoters. Nucleic Acids Res. 1990 May 25; 18(10):2891-9.
18. Mattaj I W, Dathan N A, Parry H D, Carbon P, Krol A. Changing the RNA polymerase specificity of U snRNA gene promoters. Cell. 1988 Nov. 4; 55(3):435-42. Parry H D, Tebb G, Mattaj MT. The Xenopus U2 gene PSE is a single, compact, element required for transcription initiation and 3′ end formation. Nucleic Acids Res. 1989 May 25; 17(10):3633-44.
19. Cozzarelli N R, Gerrard S P, Schlissel M, Brown D D, Bogenhagen D F. Purified RNA polymerase III accurately and efficiently terminates transcription of 5S RNA genes. Cell. 1983 October; 34(3):829-35.
20. Braglia P, Percudani R, Dieci G. Sequence context effects on oligo(dT) termination signal recognition by Saccharomyces cerevisiae RNA polymerase III. J Biol Chem. 2005 May 20; 280(20):19551-62. Epub 2005 Mar. 22.
21. D. H. Mathews, J. Sabina, M. Zuker & D. H. Turner. Expanded Sequence Dependence of Thermodynamic Parameters Improves Prediction of RNA Secondary Structure J. Mol. Biol. 288, 911-940 (1999).
22. A. F. A. Smit, R. Hubley & P. Green RepeatMasker at http://repeatmasker.org
23. Liao H, Winkfein R J, Mack G, Rattner J B, Yen T J (1995) CENP-F is a protein of the nuclear matrix that assembles onto kinetochores at late G2 and is rapidly degraded after mitosis. J Cell Biol. 130(3):507-18.
24. Gilbert N, Labuda D (1999) CORE-SINEs: eukaryotic short interspersed retroposing elements with common sequence motifs. Proc Natl Acad Sci USA. 96(6):2869-74.
25. EMBO J. 1983; 2(5):691-696. The Alu family repeat promoter has a tRNA-like bipartite structure. Paolella G, Lucero M A, Murphy M H, Baralle F E.
26. Wu L, Pan J, Thoroddsen V, Wysong D R, Blackman R K et al. (2003) Novel small-molecule inhibitors of RNA polymerase III, Eukaryot Cell. 2(2):256-64.
27. Krek A, Grun D, Poy M N, Wolf R, Rosenberg L (2005) Combinatorial microRNA target predictions. Nat Genet. 37(5):495-500.

1-21. (canceled)

22. Nucleic acid molecule comprising a nucleotide sequence that is characterized by:
being transcribed by an RNA polymerase III,
it does not undergone any polyadenylated tail addition (as for Pol II transcribed genes) and,
it is able to modulate the expression of one or more specific RNA polymerase II-transcribed target genes.

23. The nucleic acid according to claim 22, further comprising wherein said nucleotide sequence comprises a sequence of at least 50 nucleotides that is at least 70% identical to a fragment of one of the strands of the specific RNA polymerase II-transcribed target genes.

24. The nucleic acid according to claim 23, further comprising wherein said sequence of at least 50 nucleotides is in a sense or an antisense configuration with respect to the fragment of one of the strands of the specific RNA polymerase II-transcribed target genes.

25. The nucleic acid according to claim 22 comprised in one of the sequences from SEQ ID No. 51 to SEQ ID No. 84.

26. The nucleic acid according to claim 23, further comprising wherein the sequence of at least 50 nucleotides that is at least 70% identical to a fragment of one of the strands of the specific RNA polymerase II-transcribed target gene is comprised in one the underlined fragments of the sequences from SEQ ID No. 51 to SEQ ID No. 84.

27. Expression vector comprising the nucleic acid of claim 22.

28. Array for the detection of specific nucleic acid sequences containing a repertoire of nucleic acids according to claim 22.

29. A method of using the nucleic acid of claim 22, wherein the use is selected from the group consisting of: modulating the expression of RNA polymerase II transcribed genes; identifying a target sequence for treatment and/or prevention of a molecular pathology; identifying a target sequence for treatment and/or prevention of an age related pathology, including Alzheimer disease; identifying a target sequence for treatment and/or prevention of a pathology caused by an alteration of cell proliferation; and identifying a target sequence for treatment and/or prevention of a pathology that is a tumor associated pathology.

30. The nucleic acid of claim 22, further comprising wherein at least one sequence modulates the RNA polymerase III mediated expression of the nucleic acid.

31. The sequence of claim 30, wherein the nucleic acid is a promoter sequence.

32. The nucleic acid of claim 31, comprised in one of the sequences from SEQ ID No. 51 to SEQ ID No. 84.

33. A nucleic acid sequence being able to modulate the RNA polymerase III mediated expression of a nucleic acid according to claim 1, wherein the nucleic acid sequence is comprised in the bold regions of sequences from SEQ ID No. 51 to SEQ ID No. 84.

34. A method of using the nucleic acid of claim 33, wherein the use is selected from the group consisting of: modulating the expression of RNA polymerase II transcribed genes; identifying a target sequence for treatment and/or prevention of a molecular pathology; identifying a target sequence for treatment and/or prevention of an age related pathology, including Alzheimer disease; identifying a target sequence for treatment and/or prevention of a pathology caused by an alteration of cell proliferation; and identifying a target sequence for treatment and/or prevention of a pathology that is a tumor associated pathology.

35. Vector comprising the nucleic acid according to claim 33 to get expression or silencing of a RNA polymerase II transcribed specific nucleotide sequence.
